(19) United States
(12) Patent Application Publication
(10) Pub. No.: US 2021/0002232 A1
(43) Pub. Date: Jan. 7, 2021

(54) HETEROCYCLIC COMPOUNDS AS FUNGICIDES

(71) Applicant: PI INDUSTRIES LTD., Udaipur Rajasthan (IN)

(72) Inventors: Nilesh Bharat ADHAV, Maharashtra (IN); Rajesh PAWAR, Betul- MP (IN); J. YUVARAJ, Tamil Nadu (IN); Paras Raybhan BHUJADE, Maharashtra (IN); Maruti N NAIK, Karnataka (IN); Rajender Kumar POTLAPALLY, Hyderabad (IN); Sachin Nagnath GUMME, Maharashtra (IN); Santosh Shridhar AUTKAR, Maharashtra (IN); Ruchi GARG, Varanasi-UP (IN); Hagalavadi M VENKATESHA, Bengaluru-Karnataka (IN); Alexander G.M. KLAUSENER, Pulheim (DE)

(73) Assignee: PI INDUSTRIES LTD., Udaipur Rajasthan (IN)

(21) Appl. No.: 15/733,593

(22) PCT Filed: Mar. 4, 2019
(86) PCT No.: PCT/IB2019/051704

§ 371 Date: Sep. 9, 2020

(30) Foreign Application Priority Data
Mar. 9, 2018 (IN) 201811008679

(51) Int. Cl. C07D 231/12; A01N 43/56; A01N 43/82; C07D 261/08; C07D 263/32; C07D 263/34; C07D 271/06; A01N 43/653; A01N 43/76; A01N 43/80

(52) U.S. Cl. CPC C07D 231/12; A01N 43/56; A01N 43/82; C07D 261/08; A01N 43/80; C07D 263/34; C07D 271/06; A01N 43/653; A01N 43/76; C07D 263/32

(57) ABSTRACT

The present invention relates to novel heterocyclic compound of Formula I,

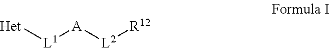

wherein, Het, L¹, A, L² and R¹² are as defined in the detailed description.

### FIELD OF THE INVENTION

The present invention relates to novel heterocyclic compounds, their N-oxides, metal complexes, isomers, polymorphs and/or the agriculturally acceptable salts thereof and to a plurality of processes for preparing the same. Further, the present invention relates to combination and compositions comprising novel heterocyclic compounds of the present invention. Still further, the present invention relates to the use of novel heterocyclic compounds of the present invention for controlling or preventing phytopathogenic fungi and to a method for controlling or preventing phytopathogenic harmful fungi.

### BACKGROUND

Oxadiazoles have already been disclosed in the literature. For example in JP56065881, JP63162680, JPS6061573, JPS6296480, JPS6051188, JP2005336101, WO2005051932, EP3165093, EP3165094, EP3167716, EP3165093, JP2017190296, U.S. Pat. No. 4,488,897, WO2015185485, WO2017055469, WO2017055473, WO2017076739, WO2017076740, WO2017081311, WO2017085098, WO2017085100, WO2017093019, WO2017093348, WO2017102006, WO2017103219, WO2017103223, WO2017109044, WO2017110861, WO2017110862, WO2017110863, WO2017110864, WO2017110865, WO2017111152, WO2017118689, WO2017148797, WO2017157962, WO2017162868, WO2017169893, WO2017174158, WO2017178549, WO2017198852, WO2017207757, WO2017211649, WO2017211650, WO2017211652, WO2017213252, WO2017220485, WO201772247, WO201776742, WO201776757, WO201776935, WO201781309, WO201781310, WO201781312, WO2018015447, WO2018015449 and WO2018015458 various oxadiazoles have be disclosed.

The oxadiazole compounds reported in the above literature have disadvantages in certain aspects, such as that they exhibit a narrow spectrum of application or they do not have satisfactory fungicidal activity, particularly at low application rates.

Therefore, it is an object of the present invention to provide compounds having improved/enhanced activity and/or a broader activity spectrum against phytopathogenic fungi.

This objective is achieved by the use of novel heterocyclic compounds of the present invention for controlling or preventing phytopathogenic fungi.

### SUMMARY

The present invention relates to novel heterocyclic compound of Formula I.

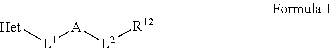

wherein, Het, L¹, A, L² and R¹² are as defined in the detailed description.

The compound of Formula I have now been found to have advantages over the compounds reported in the literature in either of improved fungicidal activity, broader spectrum biological activity, lower application rates, biological or environmental properties, and/or enhanced plant compatibility.

More specifically, the present invention further relates to a combination comprising novel heterocyclic compounds of the present invention and at least one further pesticidally active substance for effectively controlling or preventing phytopathogenic fungi which are difficult to control or prevent.

The present invention still further relates to a composition comprising novel heterocyclic compounds or novel heterocyclic compounds in combination with further pesticidally active substances.

The present invention still further relates to a method and use of novel heterocyclic compounds, or of combinations or of compositions thereof for controlling and or preventing plant diseases, particularly phytopathogenic fungi.

### DETAILED DESCRIPTION

### Definitions

The foregoing definitions provided herein for the terminologies used in the present disclosure are for illustrative purpose only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation as explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, phytophagous nematodes (Nematoda), flukes (Tematoda), acanthocephala and tapeworms (Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and biofuel crops such as, jatropha, palm trees, other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from an invertebrate parasitic pest by administering a parasiticidally effective (i.e. biologically effective) amount of a compound of the present invention, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal.

Compound of the present disclosure may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated.

The term “alkyl”, used either alone or in compound words such as “alkylthio” or “haloalkyl” or —N(alkyl) or alkylcarbonylalkyl or alkylsuphonylamino includes straight-chain or branched C₁ to C₂₄ alkyl, preferably C₁ to C₁₅ alkyl, more preferably C₁ to C₁₀ alkyl, most preferably C₁ to C₆ alkyl. Representative examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “alkenyl”, used either alone or in compound words includes straight-chain or branched C₂ to C₂₄ alkenes, preferably C₂ to C₁₅ alkenes, more preferably C₂ to C₁₀ alkenes, most preferably C₂ to C₆ alkenes. Representative examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

Representative examples of alkynes used either alone or in compound words include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. “Alkynyl” can also include moieties comprised of multiple triple bonds such as 2,5-hexadiynyl.

Cycloalkyl means alkyl closed to form a ring. Representative examples include but are not limited to cyclopropyl, cyclobutyl, cyclopentyl and cyclohexyl. This definition also applies to cycloalkyl as a part of a composite substituent, for example cycloalkylalkyl etc., unless specifically defined elsewhere.

Cycloalkenyl means alkenyl closed to form a ring including monocyclic, partially unsaturated hydrocarbyl groups. Representative examples include but are not limited to cyclopropenyl, cyclobutenyl, cyclopentenyl and cyclohexenyl. This definition also applies to cycloalkenyl as a part of a composite substituent, for example cycloalkenylalkyl etc., unless specifically defined elsewhere.

Cycloalkoxy, cycloalkenyloxy and the like are defined analogously. Non limiting examples of cycloalkoxy include cyclopropyloxy, cyclopentyloxy and cyclohexyloxy. This definition also applies to cycloalkoxy as a part of a composite substituent, for example cycloalkoxyalkyl etc., unless specifically defined elsewhere.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

Examples of “haloalkyl” include chloromethyl, bromomethyl, iodomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl” and “haloalkynyl” are defined analogously except that, instead of alkyl groups, alkenyl and alkynyl groups are present as a part of the substituent.

The term “haloalkoxy” means straight-chain or branched alkoxy groups where at least one up to all of the hydrogen atoms in these groups may be replaced by halogen atoms as specified above. Non-limiting examples of haloalkoxy include chloromethoxy, iodomethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where at least one up to all of the hydrogen atoms in these groups may be replaced by halogen atoms as specified above. Non-limiting examples of haloalkylthio include chloromethylthio, iodomethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂.

Hydroxy means —OH, amino means —NRR, wherein R can be H or any possible substituent such as alkyl. Carbonyl means —C(O)—, carbonyloxy means —OC(O)—, sulfinyl means SO, sulfonyl means S(O)₂.

The term “alkoxy” used either alone or in compound words included C₁ to C₂₄ alkoxy, preferably C₁ to C₁₅ alkoxy, more preferably C₁ to C₁₀ alkoxy, most preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkoxyalkoxy” denotes alkoxy substitution on alkoxy.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Halocycloalkyl, halocycloalkenyl, alkylcycloalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

The term “alkylthioalkyl” denotes alkylthio substitution on alkyl. Representative examples of “alkylthioalkyl” include —CH₂SCH₂, —CH₂SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. The term “cycloalkylalkylamino” denotes cycloalkyl substitution on alkyl amino.

The terms alkoxyalkoxyalkyl, alkylaminoalkyl, dialkylaminoalkyl, cycloalkylaminoalkyl, cycloalkylaminocarbonyl and the like, are defined analogously to “alkylthioalkyl” or cycloalkylalkylamino.

The term “alkoxycarbonyl” is an alkoxy group bonded to a skeleton via a carbonyl group (—CO—). This definition also applies to alkoxycarbonyl as a part of a composite substituent, for example cycloalkylalkoxycarbonyl and the like, unless specifically defined elsewhere.

The term “alkoxycarbonylalkylamino” denotes alkoxy carbonyl substitution on alkyl amino. “Alkylcarbonylalkylamino” denotes alkyl carbonyl substitution on alkyl amino. The terms alkylthioalkoxycarbonyl, cycloalkylalkylaminoalkyl and the like are defined analogously.

Examples of “alkylsulfinyl” include but are not limited to methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl and the different isomers. The term “arylsulfinyl” includes Ar—S(O), wherein Ar can be any carbocyle or heterocylcle. This definition also applies to alkylsulphinyl as a part of a composite substituent, for example haloalkylsulphinyl etc., unless specifically defined elsewhere.

Examples of “alkylsulfonyl” include but are not limited to methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl and the different isomers. The term “arylsulfonyl” includes Ar—S(O)₂, wherein Ar can be any carbocyle or heterocylcle. This definition also applies to alkylsulphonyl as a part of a composite substituent, for example alkylsulphonylalkyl etc., unless defined elsewhere.

“Alkylamino”, “dialkylamino”, and the like, are defined analogously to the above examples.

The terms “carbocycle” or “carbocyclic” or “carbocyclyl” include “aromatic carbocyclic ring system” and “nonaromatic carbocylic ring system” or polycyclic or bicyclic (spiro, fused, bridged, nonfused) ring compounds in which the ring may be aromatic or non-aromatic (where aromatic indicates that the Hueckel rule is satisfied and non-aromatic indicates that the Hueckel rule is not statisfied).

The terms “heterocycle” or “heterocyclic” or “heterocyclyl” include “aromatic heterocycle or heteroaryl ring system” and “nonaromatic heterocycle ring system” or polycyclic or bicyclic (spiro, fused, bridged, nonfused) ring compounds in which the ring may be aromatic or non-aromatic, wherein the heterocycle ring contains at least one heteroatom selected from N, O, S(O)₀₋₂, and or C ring member of the heterocycle may be replaced by C(═O), C(═S), C(═CR*R*) and C═NR*, * indicates integers (where aromatic heterocycle or heteroaryl ring indicates that the Hueckel rule is satisfied).

The term “non-aromatic heterocycle” means three- to fifteen-membered, preferably three- to twelve-membered, saturated or partially unsaturated heterocycles containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; for example (but not limited to) oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl and cycloserines. This definition also applies to heterocyclyl as a part of a composite substituent, for example heterocyclylalkyl etc., unless specifically defined elsewhere.

The term “heteroaryl” means 5 or 6-membered, fully unsaturated monocyclic ring system containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazol-1-yl, 1-imidazolyl, 1,2,3-triazol-1-yl and 1,3,4-triazol-1-yl.

6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: for example (but not limited to) indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl; benzofused 6-membered heteroaryl which contains one to three nitrogen atoms: for example (but not limited to) quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

This definition also applies to heteroaryl as a part of a composite substituent, for example heteroarylalkyl etc., unless specifically defined elsewhere.

The term “aromatic” indicates that the Hueckel rule is satisfied and the term “non-aromatic” indicates that the Hueckel rule is not satisfied.

“Trialkylsilyl” includes 3 branched and/or straight-chain alkyl radicals attached to and linked through a silicon atom such as trimethylsilyl, triethylsilyl and t-butyl-dimethylsilyl. “Halotrialkylsilyl” denotes at least one of the three alkyl radicals is partially or fully substituted with halogen atoms which may be the same or different. “Alkoxytrialkylsilyl” denotes at least one of the three alkyl radicals is substituted with one or more alkoxy radicals which may be the same or different. “Trialkylsilyloxy” denotes a trialkylsilyl moiety attached through oxygen.

Examples of “alkylcarbonyl” include C(O)CH₃, C(O)CH₂CH₂CH₃ and C(O)CH(CH₃)₂. Examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Examples of “dialkylaminocarbonyl” include (CH₃)₂NC(═O), (CH₃CH₂)₂NC(═O), CH₃CH₂(CH₃)NC(═O), CH₃CH₂CH₂(CH₃)NC(═O) and (CH₃)₂CHN(CH₃)C(═O). Examples of “alkoxyalkylcarbonyl” include CH₃OCH₂C(═O), CH₃OCH₂CH₂C(═O), CH₃CH₂OCH₂C(═O), CH₃CH₂CH₂CH₂OCH₂C(═O) and CH₃CH₂OCH₂CH₂C(═O). Examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O). The term haloalkylsufonylaminocarbonyl, alkylsulfonylaminocarbonyl, alkylthioalkoxycarbonyl, alkoxycarbonylakyl amino and the like are defined analogously

Examples of “alkylaminoalkylcarbonyl” include CH₃NHCH₂C(═O), CH₃NHCH₂CH₂C(═O), CH₃CH₂NHCH₂C(═O), CH₃CH₂CH₂CH₂NHCH₂C(═O) and CH₃CH₂NHCH₂CH₂C(═O).

The term “amide” means A-R′C═ONR″-B, wherein R′ and R″ indicate substituents and A and B indicate any group.

The term “thioamide” means A-R′C═SNR″-B, wherein R′ and R″ indicate substituents and A and B indicate any group.

The total number of carbon atoms in a substituent group is indicated by the “Cᵢ-Cⱼ” prefix where i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃CH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂CH₂ and CH₃CH₂OCH₂CH₂. In the above recitations, when a compound of Formula I is comprised of one or more heterocyclic rings, all substituents are attached to these rings through any available carbon or nitrogen by replacement of a hydrogen on said carbon or nitrogen.

When a compound is substituted with a substituent bearing a subscript that indicates that the number of said substituents can exceed 1, said substituents (when they exceed 1) are independently selected from the group of defined substituents. Further, when the subscript m in (R), indicates an integer ranging from for example 0 to 4, then the number of substituents may be selected from the integers from 0 and 4 inclusive.

When a group contains a substituent which can be hydrogen, then, when this substituent is taken as hydrogen, it is recognized that said group is being un-substituted.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned in the description and the foregoing claims though might form a critical part of the present invention of the present disclosure, any deviation from such numerical values shall still fall within the scope of the present disclosure if that deviation follows the same scientific principle as that of the present invention disclosed in the present disclosure.

The term “pest” for the purpose of the present disclosure includes but is not limited to fungi, stramenopiles (oomycetes), bacteria, nematodes, mites, ticks, insects and rodents.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

For the purpose of the present disclosure the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include but are not limited to agricultural crops such as wheat, rye, barley, triticale, oats or rice; beet, e.g. sugar beet or fodder beet; fruits and fruit trees, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit and citrus trees, such as oranges, lemons, grapefruits or mandarins; any horticultural plants, vegetables, such as spinach, lettuce, asparagus, cabbages, carrots, onions, tomatoes, potatoes, cucurbits or paprika; lauraceous plants, such as avocados, cinnamon or camphor; cucurbitaceae; oleaginous plants; energy and raw material plants, such as cereals, corn, soybean, other leguminous plants, rape, sugar cane or oil palm; tobacco; nuts; coffee; tea; cacao; bananas; peppers; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, the plant for the purpose of the present invention include but is not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for use of human and animals.

The term “plant parts” is understood to mean all parts and organs of plants above and below the ground. For the purpose of the present disclosure the term plant parts includes but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxillary buds, meristems, nodes and internodes.

The term “locus thereof” includes soil, surroundings of plant or plant parts and equipment or tools used before, during or after sowing/planting a plant or a plant part.

Application of the compound of the present disclosure or the compound of the present disclosure in a composition optionally comprising other compatible compounds to a plant or a plant material or locus thereof include application by a technique known to a person skilled in the art which include but is not limited to spraying, coating, dipping, fumigating, impregnating, injecting and dusting.

The term “applied” means adhered to a plant or plant part either physically or chemically including impregnation.

Accordingly, novel heterocyclic compound of the present invention are represented by Formula I and include N-oxides, metal complexes, isomers, polymorphs or the agriculturally acceptable salts thereof.

The present invention relates to compound of the Formula I,

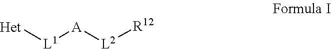

wherein;
Het is selected from the group consisting of Het-1 to Het-18

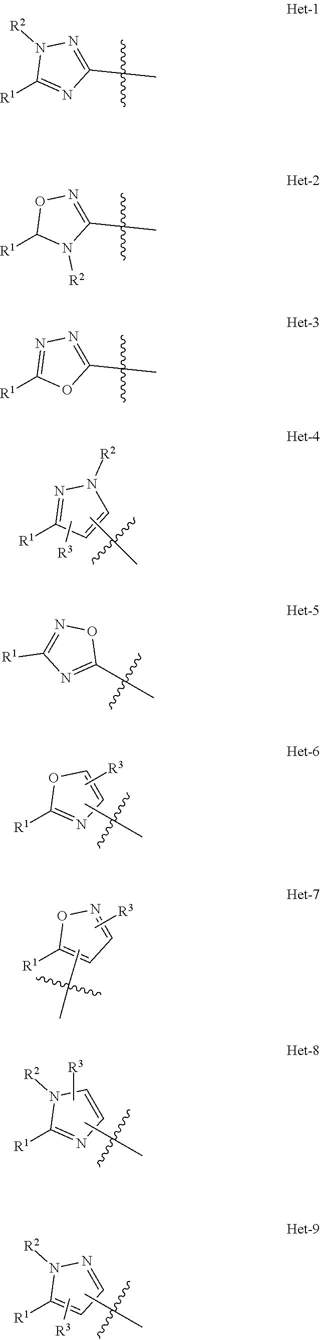

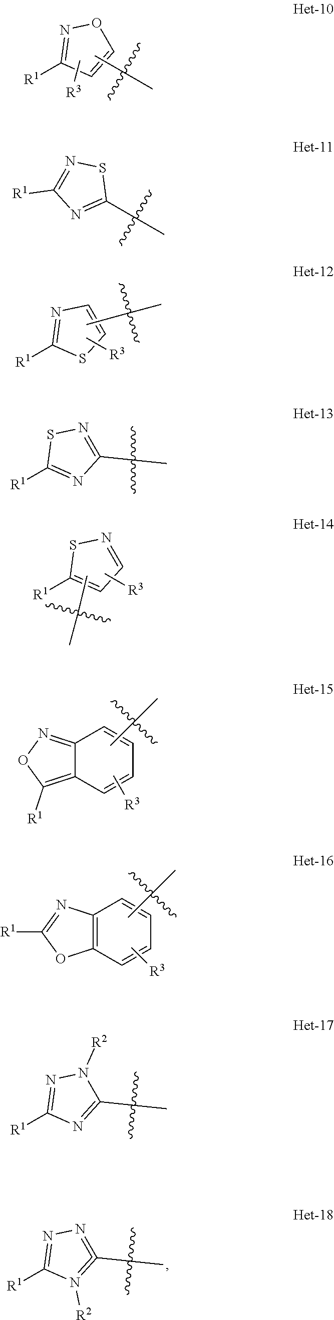

wherein, the expression “ [symbol] ” indicates the point of attachment to L¹;
R¹ is C₁-C₆ haloalkyl;
R² is independently selected from the group consisting of hydrogen, C₁-C₆-alkyl, C₁-C₆-haloalkyl, C₃-C₆-cycloalkyl, C₃-C₆-halocycloalkyl, C₁-C₆-alkoxy, C₁-C₆-haloalkoxy, C₃-C₆-cycloalkylalkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, and C₃-C₆-halocycloalkylalkyl;
R³ is independently selected from the group consisting of hydrogen, halogen, C₁-C₆-alkyl, C₁-C₆-haloalkyl, C₃-C₆-cycloalkyl, C₁-C₆-alkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, and C₁-C₆-haloalkoxy;
   Li is a direct bond, —CR⁴R⁵—, —C(═O)—, —CH₂C(═O)—, —O—, —S(═O)₀₋₂—, and —NR⁶—, wherein, an expression “-” at the start and the end of the group indicates the point of attachment to either Het or A;
wherein, R⁴ and R⁵ are independently selected from hydrogen, halogen, cyano, C₁-C₄-alkyl, C₁-C₄-haloalkyl, C₃-C₆-cycloalkyl, C₃-C₆-halocycloalkyl, C₁-C₄-alkoxy or C₁-C₄-haloalkoxy, or
R⁴ and R⁵ together with the atoms to which they are attached may form 3- to 6-membered non aromatic carbocylic ring or heterocyclic ring which may be optionally substituted with halogen, C₁-C₂-alkyl, C₁-C₂-haloalkyl or C₁-C₂-alkoxy; and
R⁶ is independently selected from hydrogen, C₁-C₆-alkyl, C₁-C₆-haloalkyl, C₃-C₆-cycloalkyl, C₃-C₆-halocycloalkyl, C₁-C₆-alkoxy, C₁-C₆-haloalkoxy, C₃-C₆-cycloalkylalkyl, and C₃-C₆-halocycloalkylalkyl;
   A is phenyl or a 5- or 6-membered heteroaryl ring; wherein the heteroatoms of the heteroaryl are selected from N, O and S; and wherein the phenyl or the 5- or 6-membered heteroaryl may be unsubstituted or substituted with one or more identical or different R^{A} groups,
wherein, R^{A} is hydrogen, halogen, cyano, nitro, sulfanyl, amino, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₂-C₆-alkenyloxy, C₂-C₆-haloalkenyloxy, C₂-C₆-alknyloxy, C₂-C₆-haloalknyloxy, C₁-C₆-haloalkoxy, C₃-C₈-cycloalkoxy, C₁-C₆-haloalkoxycarbonyl, C₁-C₆-alkylthio, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, C₁-C₆-dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, C₁-C₆-dialkylaminocarbonyl, C₁-C₆-alkoxycarbonyloxy, C₁-C₆-alkylaminocarbonyloxy, or C₁-C₆-dialkylaminocarbonyloxy,
   and wherein R^{A} may be optionally substituted with one or more identical or different R^{a} selected from halogen, cyano, nitro, sulfanyl, amino, hydroxy, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₁-C₆-alkoxy-C₁-C₄-alkyl, C₃-C₈-halocycloalkyl, C₁-C₆-alkylamino, di-C₁-C₆-alkylamino or C₃-C₈-cycloalkylamino;
      L² is a direct bond or is selected from the group of —CR⁷R⁸—; —C(═O)—; —C(═S)—; —O—; —S(═O)₀₋₂—; —NR¹⁰—;

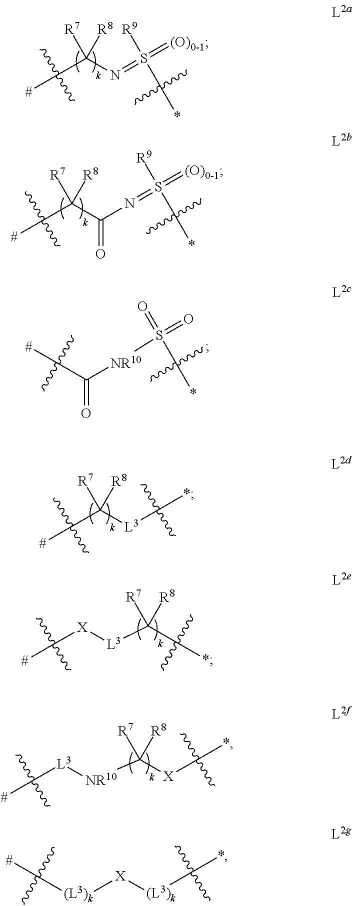

wherein X is a direct bond or —NR¹⁰—, or —O—, or —S(O)₀₋₂— or —C(═NOR¹¹)—; or a 5-membered heteroaryl ring which is substituted or unsubstituted with one or more identical or different R^{L} wherein R^{L} is independently selected from halogen, amino, C₁-C₆-alkylamino, di-C₁-C₆-alkylamino, C₁-C₆-alkyl, C₁-C₆-alkoxy, C₂-C₆-alkenyl, C₂-C₆-alkynyl, C₃-C₈-cycloalkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, or C₃-C₈-cycloalkoxy; and wherein R^{L} may be optionally substituted with one or more identical or different Rⁱ; wherein, Rⁱ is halogen, cyano, C₁-C₆-alkyl, C₁-C₆-haloalkyl, C₁-C₆-alkoxy, C₁-C₆-haloalkoxy, or C₃-C₈-cycloalkyl,
wherein,
k is an integer ranging from 0 to 4; expressions “-”, “#” and “*” indicate point of attachments to either A or R¹²;
L³ is a direct bond, —CR⁷R⁸—, —CH₂C(O)—, —C(═O)—, —C(═S)—, —O—, —S(═O)₀₋₂—, —S(O)₀₋₁(═N—R¹⁰)—, —S(═N—CN)—, —S(═N—NO₂)—, —S(═N—COR⁷)—, —S(═N—COOR¹¹)—, —S(═N—(S(═O)₂R⁹))—, —NR¹⁰—, —NR¹⁰(C(═O))O—, —CR⁷(═N)O—,
   wherein, R⁷ and R⁸ are independently hydrogen, halogen, cyano, C₁-C₆-alkyl, C₁-C₆-haloalkyl, C₁-C₆-alkoxy, C₂-C₆-alkenyl, C₂-C₆-alkynyl, C₃-C₈-cycloalkyl, C₁-C₆-alkylthio, C₃-C₈-cycloalkenyl, phenyl-C₁-C₆-alkyl, heteroaryl-C₁-C₆-alkyl, phenyl, naphthyl or a 3- to 10-membered saturated, partially unsaturated or aromatic mono- or bicyclic carbocyclic ring or heterocyclic ring, wherein the ring members of the heteroaryl of the heteroaryl-C₁-C₆-alkyl and the heterocyclic ring include C, N, O and S(O)₀₋₂ and the C ring members of the carbocylic ring or the heterocyclic ring may be replaced by one or more C(═O) and C(═S); and wherein R⁷ and R⁸ are independently unsubstituted or substituted with one or more identical or different R^{7a} selected from the group consisting of halogen, cyano, nitro, hydroxyl, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, amino-C₁-C₆-alkyl, di-C₁-C₆-alkylamino, NHSO₂—C₁-C₆-alkyl, —C(═O)—C₁-C₆-alkyl, C(═O)—C₁-C₆-alkoxy, C₁-C₆-alkylsulfonyl, hydroxy-C₁-C₆-alkyl, —C(═O)—NH₂, C(═O)—NH(C₁-C₆-alkyl), C₁-C₆-alkylthio-C₁-C₆-alkyl, C₁-C₆-alkylamino-C₁-C₆-alkyl, di-C₁-C₆-alkylamino-C₁-C₆-alkyl, aminocarbonyl-C₁-C₆-alkyl or C₁-C₆-alkoxy-C₁-C₆-alkyl, or
   R⁷ and R⁸ together with the carbon atom to which they are bound form C(═O) or a vinyl group or a saturated, monocyclic 3- to 7-membered heterocycle or carbocycle, wherein the ring members of heterocyclic include C, N, O and S(O)₀₋₂; and wherein the vinyl group, the heterocyclic ring or the carbocyclic ring is unsubstituted or substituted with one or more identical or different R^{7b}, wherein R^{7b} is halogen, cyano, nitro, hydroxyl, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, SO₂—C₁-C₆-alkyl, NHSO₂—C₁-C₆-alkyl, —C(═O)—C₁-C₆-alkyl, C(═O)—C₁-C₆-alkoxy, C₁-C₆-alkylsulfonyl, SO₂—C₆H₄CH₃, or SO₂-aryl;
   R⁹ is independently selected from the group consisting of hydrogen; NR^{g}R^{h}, wherein, R^{g} and R^{h} independently represent hydrogen, hydroxyl, cyano, C₁-C₄-alkyl, C₁-C₄-haloalkyl, C₁-C₄-alkoxy or C₃-C₈-cycloalkyl; (C═O)—Rⁱ, wherein, Rⁱ represents hydrogen, halogen, cyano, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, C₃-C₆-halocycloalkyl, C₁-C₄-alkoxy, and C₁-C₄-haloalkoxy; C₁₋₈-alkyl-S(O)₀₋₂R^{j}, wherein R^{j} represents hydrogen, halogen, cyano, C₁-C₆-alkyl, C₁-C₆-haloalkyl, C₁-C₆-alkoxy, C₁-C₆-haloalkoxy, C₃-C₈-cycloalkyl; C₁-C₆-alkyl-(C═O)—Rⁱ, CRⁱ═NR^{g}, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₁-C₆-haloalkoxy, C₃-C₈-cycloalkyl, C₄-C₈-cycloalkenyl, C₇-C₁₉-aralkyl, bicyclic C₅-C₁₂-alkyl, C₇-C₁₂-alkenyl, fused or non-fused or bicyclic C₃-C₁₅-carbocyclic ring or ring system; wherein one or more C atoms of the carbocyclic ring or ring system may be replaced by N, O, S(═O)₀₋₂, S(═O)₀₋₁(═NR¹⁰), C(═O), C(═S), C(═CR⁷R⁸) and C═NR¹⁰,
      wherein, R⁹ may optionally be substituted with one or more identical or different substituents selected from hydrogen, halogen, cyano, nitro, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, C₁-C₆-haloalkoxycarbonyl, C₁-C₆-alkylthio, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, di-C₁-C₆-alkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, di-C₁-C₆-alkylaminocarbonyl, C₁-C₆-alkoxycarbonyloxy, C₁-C₆-alkylaminocarbonyloxy, di C₁-C₆-alkylaminocarbonyloxy, 5- to 11-membered spirocyclic ring, or 3- to 6-membered carbocyclic or heterocyclic ring;
   R¹⁰ independently of each other are hydrogen, C₁-C₆-alkyl, C₁-C₆-haloalkyl, C₁-C₆-alkyl-CN, C₁-C₆-alkoxy, C₂-C₆-alkenyl, C₂-C₆-alkynyl, C₃-C₈-cycloalkyl, C₃-C₈-cycloalkenyl, phenyl-C₁-C₆-alkyl, (C═O)—C₁-C₆-alkyl, C(═O)—C₁-C₆-alkoxy, 5- or 6-membered heteroaryl-C₁-C₆-alkyl, phenyl, naphthyl or a 3- to 10-membered saturated, partially unsaturated or aromatic mono- or bicyclic carbocyclic ring or heterocyclic ring, wherein the ring members of the heteroaryl of heteroaryl-C₁-C₆-alkyl and the mono- or bicyclic heterocycle are selected from C, N, O and S and wherein one or more C ring members of the carbocylic ring or the heterocyclic ring may be replaced by one or more groups selected from C(═O) and C(═S); and wherein R¹⁰ is unsubstituted or substituted with one or more identical or different R^{10a}; wherein, R^{10a} is halogen, cyano, oxo, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, —C(═O)—C₁-C₆-alkyl, C(═O)—C₁-C₆-alkoxy, C₁-C₆-alkylsulfonyl, hydroxy-C₁-C₆-alkyl, —C(═O)—NH₂, C(═O)—NH(C₁-C₆-alkyl), C₁-C₆-alkylthio-C₁-C₆-alkyl, amino-C₁-C₆-alkyl, C₁-C₆-alkylamino-C₁-C₆-alkyl, di-C₁-C₆-alkylamino-C₁-C₆-alkyl, aminocarbonyl-C₁-C₆-alkyl or C₁-C₆-alkoxy-C₁-C₆-alkyl;
   R¹¹ is independently selected from hydrogen, C₁-C₆-alkyl, C₁-C₆-haloalkyl, C₂-C₆-alkenyl, C₂-C₆-alkynyl, C₃-C₈-cycloalkyl, C₁-C₆-alkylthio, C₃-C₈-cycloalkenyl, phenyl-C₁-C₆-alkyl, heteroaryl-C₁-C₆-alkyl, phenyl, naphthyl or a 3- to 10-membered saturated, partially unsaturated or aromatic mono- or bicyclic carbocyclic or heterocyclic ring, wherein the ring members of the heteroaryl in the heteroaryl-C₁-C₆-alkyl and the heterocyclic ring include C, N, O and S(O)₀₋₂ and the C ring members of the carbocyclic ring or the heterocyclic ring may be replaced by one or more C(═O) and C(═S); and wherein R¹¹ is independently unsubstituted or substituted with one or more identical or different R^{11a} selected from the group consisting of halogen, cyano, nitro, hydroxyl, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, amino-C₁-C₆-alkyl, or di-C₁-C₆-alkylamino; or
   A, L² and R¹² together form a fragment A¹

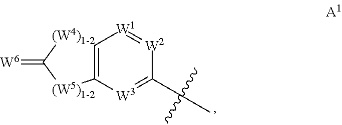

wherein W¹, W², W³, W⁴, and W⁵, independently are C or N, provided all are not N simultaneously; W⁶ is O or S; the expression

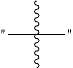

indicates the point of attachment to Het; and the fragment A¹ is substituted or unsubstituted with one or more identical or different R^{A};
R¹² is NR^{12a}R^{12b}, OR³, NR¹⁴NR^{12a}R^{12b}, R¹⁵, S(O)₀₋₂R¹⁶, COOR¹³, CONR^{12a}R^{12b}, COR¹⁵, NR^{12a}OR¹³,
   wherein, R^{12a}, R^{12b}, and R¹⁴ are hydrogen, C₁-C₆-alkyl, C₁-C₆-haloalkyl, C₁-C₆-alkoxy, C₂-C₆-alkenyl, C₂-C₆-alkynyl, C₃-C₈-cycloalkyl, C₃-C₈-cycloalkenyl, phenyl-C₁-C₆-alkyl, (C═O)—C₁-C₆-alkyl, C(═O)—C₁-C₆-alkoxy, C₁-C₆-alkoxyimino-C₁-C₆-alkyl, C₂-C₆-alkenyloxyimino-C₁-C₆-alkyl, C₂-C₆-alkynyloxyimino-C₁-C₆-alkyl, aminocarbonyl-C₁-C₆-alkyl, C₃-C₈-cycloalkyl-C₁-C₆-alkyl, heterocyclyl-C₁-C₆-alkyl, 5- or 6-membered heteroaryl-C₁-C₆-alkyl, phenyl, naphthyl or a 3- to 10-membered saturated, partially unsaturated or aromatic mono- or bicyclic carbocyclic ring of heterocyclic, wherein the ring members of said heteroaryl of heteroaryl-C₁-C₆-alkyl and said mono- or bicyclic heterocyclic ring are selected from C, N, O and S and wherein one or more C ring members of the carbocylic ring or the heterocyclic ring may be replaced by one or more groups selected from C(═O) and C(═S); and wherein R^{12a} and R^{12b} are unsubstituted or substituted with one or more identical or different R^{12c}; wherein, R^{12c} is halogen, cyano, nitro, oxo, hydroxy, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, —C(═O)—C₁-C₆-alkyl, C(═O)—C₁-C₆-alkoxy, C₁-C₆-alkylsulfonyl, hydroxy-C₁-C₆-alkyl, —C(═O)—NH₂, C(═O)—NH(C₁-C₆-alkyl), C₁-C₆-alkylthio-C₁-C₆-alkyl, amino-C₁-C₆-alkyl, C₁-C₆-alkylamino-C₁-C₆-alkyl, di-C₁-C₆-alkylamino-C₁-C₆-alkyl, aminocarbonyl-C₁-C₆-alkyl or C₁-C₆-alkoxy-C₁-C₆-alkyl; or
   R^{12a} and R^{12b} together with the nitrogen atom to which they are bound form a saturated or partially unsaturated mono- or bicyclic 3- to 10-membered heterocyclic ring, wherein the ring members heterocyclic ring include beside one nitrogen atom, C, N, O and S(O)₀₋₂; and wherein one or more C atom of the heterocyclic ring may be replaced by one or more C(═O) and C(═S); and wherein the heterocyclic ring is unsubstituted or substituted with one or more identical or different groups R^{12d}; wherein R^{12d} is halogen, cyano, nitro, oxo, hydroxy, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, (C═O)—C₁-C₆-alkyl, C(═O)—C₁-C₆-alkoxy, C₁-C₆-alkylsulfonyl, hydroxyC₁-C₆-alkyl, C(═O)—NH₂, C(═O)—NH(C₁-C₆-alkyl), C₁-C₄-alkylthio-C₁-C₆-alkyl, amino-C₁-C₆-alkyl, C₁-C₆-alkylamino-C₁-C₆-alkyl, diC₁-C₆-alkylamino-C₁-C₆-alkyl, aminocarbonyl-C₁-C₆-alkyl or C₁-C₆-alkoxy-C₁-C₆-alkyl;
   R¹³, R¹⁵ and R¹⁶ is hydrogen, C₁-C₆-alkyl, C₁-C₆-alkoxy, C₁-C₆-alkoxy-C₁-C₆-alkyl, C₂-C₆-alkenyl, C₂-C₆-alkynyl, C₃-C₈-cycloalkyl, C₃-C₈-cycloalkenyl, —CH═N—O—C₁-C₆-alkyl, C(═O)—(C₁-C₆-alkyl), C(═O)—(C₁-C₆-alkoxy), C(═O)—(C₃-C₈-cycloalkyl), C(═O)-(phenyl), C(═O)-(heteroaryl), C₁-C₆-alkyl-C(═O)—(C₁-C₆-alkyl), C₁-C₆-alkyl-C(═O)—(C₁-C₆-alkoxy), C₁-C₆-alkoxyimino, C₁-C₆-alkoxyimino-C₁-C₆-alkyl, C₂-C₆-alkenyloxyimino-C₁-C₆-alkyl, C₂-C₆-alkynyloxyimino-C₁-C₆-alkyl, C₃-C₈-cycloalkyl-C₁-C₆-alkyl, aminocarbonyl-C₁-C₆-alkyl, C₁-C₆-alkylaminocarbonyl, C₁-C₆-alkylaminocarbonyl-C₁-C₆-alkyl, C₁-C₆-alkyl-NH—C(═O)(C₁-C₆-alkyl), C₁-C₆-alkyl-NH—C(═O)(C₃-C₈-cycloalkyl), C₁-C₆-alkyl-NH—C(═O)(phenyl), C₁-C₆-alkyl-NH—C(═O)—N(heteroaryl), C₁-C₆-alkyl-C(═O)—NH(C₁-C₆-alkyl), C₁-C₆-alkyl-C(═O)—N(C₁-C₆-alkyl)₂, di-C₁-C₆-alkyl-C(═O)—NH(C₃-C₆-cycloalkyl), C₁-C₆-alkyl-C(═O)—N(C₁-C₆-alkyl)(C₃-C₈-cycloalkyl), C₁-C₆-alkyl-C(═O)—NH(phenyl), C₁-C₆-alkyl-C(═O)—N(C₁-C₆-alkyl)(phenyl), C₁-C₆-alkyl-C(═O)—NH(heteroaryl), C₁-C₆-alkyl-C(═O)—N(C₁-C₆-alkyl)(heteroaryl), C₁-C₆-alkyl-C(═O)—NH(C₁-C₆-alkyl-C₃-C₈-cycloalkyl), C₁-C₆-alkyl-C(═O)—N(C₁-C₆-alkyl)(C₁-C₆-alkyl-C₃-C₈-cycloalkyl), C₁-C₆-alkyl-C(═O)—NH(C₁-C₆-alkyl-phenyl), C₁-C₆-alkyl-C(═O)—N(C₁-C₆-alkyl)(C₁-C₆-alkyl-phenyl), C₁-C₆-alkyl-C(═O)—NH(C₁-C₆-alkyl-heteroaryl), C₁-C₆-alkyl-C(═O)—N(C₁-C₆-alkyl)(C₁-C₆-alkyl-heteroaryl), C₁-C₆-alkylaminocarbonyl-C₃-C₈-cycloalkyl, C₃-C₈-cycloalkyl-C₁-C₆-alkyl, phenyl-C₁-C₆-alkyl, heteroaryl-C₁-C₆-alkyl, C₃-C₈-cycloalkyl-C₁-C₆-alkoxy, phenyl-C₁-C₆-alkoxy, heteroaryl-C₁-C₆-alkoxy, C₁-C₆-alkoxy-C₁-C₆-alkyl, C₃-C₈-cycloalkoxy-C₁-C₆-alkyl, phenoxy-C₁-C₆-alkyl, heteroaryloxy-C₁-C₆-alkyl, phenyl, naphthyl or a 3- to 10-membered saturated, partially unsaturated or aromatic mono- or bicyclic carbocyclic ring or heterocyclic ring, wherein the ring member atoms of said heteroaryls or said mono- or bicyclic heterocyclic ring include C, N, O and S(O)₀₋₂; wherein C ring member of the carbocyclic ring or the heterocyclic ring may be replaced by one or more C(═O) and C(═S); and wherein R¹³, R¹⁵ and R¹⁶ may be substituted or unsubstituted with one or more identical or different R^{15a},
   R^{15a} is halogen, cyano, hydroxy, oxo, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, (C═O)—(C₁-C₆-alkyl), C(═O)—(C₁-C₆-alkoxy), C₁-C₆-alkylsulfonyl, C₁-C₆-alkoxy-C₁-C₆-alkyl, hydroxy-C₁-C₆-alkyl, C(═O)—NH₂, C(═O)—NH(C₁-C₆-alkyl), C(═O)—N(C₁-C₆-alkyl)₂, —NH(C₁-C₆-alkyl), —N(C₁-C₆-alkyl)₂, C₁-C₆-alkylthio-C₁-C₆-alkyl, amino-C₁-C₆-alkyl, C₁-C₆-alkylamino-C₁-C₆-alkyl, di-C₁-C₆-alkylamino-C₁-C₆-alkyl, or aminocarbonyl-C₁-C₆-alkyl; or
   R¹⁵ is a 3- to 10-membered saturated, partially unsaturated or aromatic mono- or bicyclic carbocyclic or heterocyclic ring, wherein the ring members of the heterocyclic ring include C, N, O and S(O)₀₋₂ and the C ring members of the carbocyclic ring or the heterocyclic ring may be replaced by one or more C(═O) and C(═S); and wherein the carbocyclic ring and the heterocyclic ring are independently unsubstituted or substituted with one or more identical or different R^{15a}; or
   R¹⁵ is phenyl or 5- or 6-membered heteroaryl, wherein the ring members of the heteroaryl ring include C, N, O and S; and wherein the phenyl and the heteroaryl rings are independently unsubstituted or substituted with one or more identical or different R^{15a};
      or N-oxides, metal complexes, isomers, polymorphs or the agriculturally acceptable salts thereof.

Particularly, the present invention relates to a compound of Formula I,

wherein,

### Het is Het-1, Het-2, Het-3, Het-4, Het-5, Het-6, Het-7 and Het-9;

R¹ is independently selected from the group consisting of CF₃, CHF₂, CF₂Cl, CF₂CF₃CH₂F, CH₂CF₃, CHClCF₃, CCl₂CF₃;
L¹ is direct bond;
A is phenyl; and
L² is S(═O)₂, C(═O), L^{2a}, L^{2b}, L^{2c}, L^{2f} and L^{2g}.

The representative compounds of Formula I of the present invention include N-((4-fluorophenyl)(methyl)(oxo)-λ⁶-sulfaneylidene)-4-(5-(trifluoromethyl)-4,5-dihydro-1,2,4-oxadiazol-3-yl)benzamide; N-(4-(5-(difluoromethyl)-1,2,4-oxadiazol-3-yl)benzyl)-N-methoxybenzamide; N-(4-(5-(trifluoromethyl)-1H-1,2,4-triazol-3-yl)benzyl)pivalamide; 5-(difluoromethyl)-3-(4-(phenylsulfonyl)phenyl)-1,2,4-oxadiazole; N-((4-fluorophenyl)(methyl)(oxo)-λ⁶-sulfaneylidene)-4-(5-(trifluoromethyl)-4,5-dihydro-1,2,4-oxadiazol-3-yl)benzamide; N-((4-fluorophenyl)(methyl)(oxo)-λ⁶-sulfaneylidene)-4-(5-(trifluoromethyl)-1H-1,2,4-triazol-3-yl)benzamide; N-(methyl(oxo)(phenyl)-λ⁶-sulfaneylidene)-4-(5-(trifluoromethyl)-1H-1,2,4-triazol-3-yl)benzamide; 3-(4-(phenylsulfonyl)phenyl)-5-(trifluoromethyl)-1H-1,2,4-triazole; N-(methyl(oxo)(phenyl)-λ⁶-sulfaneylidene)-4-(5-(trifluoromethyl)-4,5-dihydro-1,2,4-oxadiazol-3-yl)benzamide; N-methyl-N-(2-phenoxyethyl)-4-(5-(trifluoromethyl)-1H-1,2,4-triazol-3-yl)benzamide; 3-(4-(phenylsulfonyl)phenyl)-5-(trifluoromethyl)-4,5-dihydro-1,2,4-oxadiazole; N-methyl-N-(2-phenoxyethyl)-4-(5-(trifluoromethyl)-4,5-dihydro-1,2,4-oxadiazol-3-yl)benzamide; (4-methoxyphenyl)(methyl)((4-(5-(trifluoromethyl)-1H-1,2,4-triazol-3-yl)phenyl)imino)-λ⁶-sulfanone; (4-methoxyphenyl)(methyl)((4-(5-(trifluoromethyl)-4,5-dihydro-1,2,4-oxadiazol-3-yl)phenyl)imino)-λ⁶-sulfanone; 4-(5-(difluoromethyl)-1,2,4-oxadiazol-3-yl)-N-methyl-N-(2-phenoxyethyl)benzamide; 4-(5-(difluoromethyl)-4,5-dihydro-1,2,4-oxadiazol-3-yl)-N-((4-fluorophenyl)(methyl)(oxo)-λ⁶-sulfaneylidene)benzamide; 2-phenyl-N-(4-(5-(trifluoromethyl)-4,5-dihydro-1,2,4-oxadiazol-3-yl)phenyl)propanamide; N-methyl-N-(4-(5-(trifluoromethyl)-4,5-dihydro-1,2,4-oxadiazol-3-yl)benzyl)cyclopropanecarboxamide; N-methyl-N-(4-(5-(trifluoromethyl)-4,5-dihydro-1,2,4-oxadiazol-3-yl)benzyl)cyclobutanecarboxamide; 2-phenyl-N-(4-(5-(trifluoromethyl)-4,5-dihydro-1,2,4-oxadiazol-3-yl)phenyl)acetamide; methyl(pyridin-2-yl)((4-(5-(trifluoromethyl)-4,5-dihydro-1,2,4-oxadiazol-3-yl)phenyl)imino)-λ⁶-sulfanone; N-((4-methoxyphenyl)(methyl)(oxo)-λ⁶-sulfaneylidene)-4-(5-(trifluoromethyl)-4,5-dihydro-1,2,4-oxadiazol-3-yl)benzamide; methyl((4-(5-(trifluoromethyl)-4,5-dihydro-1,2,4-oxadiazol-3-yl)phenyl)imino)(4-(trifluoromethyl)phenyl)-λ⁶-sulfanone; ((4-(5-(difluoromethyl)-4,5-dihydro-1,2,4-oxadiazol-3-yl)benzyl)imino)(methyl)(phenyl)-λ⁶-sulfanone; N-(2,4-difluorophenyl)-4-(5-(trifluoromethyl)-4,5-dihydro-1,2,4-oxadiazol-3-yl)benzamide; N-(4-chloro-2-fluorophenyl)-4-(5-(trifluoromethyl)-4,5-dihydro-1,2,4-oxadiazol-3-yl)benzamide; methyl(p-tolyl)((4-(5-(trifluoromethyl)-4,5-dihydro-1,2,4-oxadiazol-3-yl)benzyl)imino)-λ⁶-sulfanone; N-(4-(5-(difluoromethyl)-1,2,4-oxadiazol-3-yl)phenyl)-2-phenylacetamide; 1-methyl-5-(4-(phenylsulfonyl)phenyl)-3-(trifluoromethyl)-1H-1,2,4-triazole; (2-fluorophenyl)(methyl)((4-(1-methyl-3-(trifluoromethyl)-1H-1,2,4-triazol-5-yl)phenyl)imino)-λ⁶-sulfanone; 2-phenyl-N-(4-(5-(trifluoromethyl)-1H-1,2,4-triazol-3-yl)phenyl)acetamide; N-methyl-N-(4-(5-(trifluoromethyl)-1H-1,2,4-triazol-3-yl)benzyl)cyclopropanecarboxamide; N-methyl-N-(4-(5-(trifluoromethyl)-1H-1,2,4-triazol-3-yl)benzyl)cyclobutanecarboxamide; 2-phenyl-N-(4-(5-(trifluoromethyl)-1H-1,2,4-triazol-3-yl)phenyl)propanamide; methyl((4-(5-(trifluoromethyl)-1H-1,2,4-triazol-3-yl)phenyl)imino)(4-(trifluoromethyl)phenyl)-λ⁶-sulfanone; methyl(pyridin-2-yl)((4-(3-(trifluoromethyl)-1H-1,2,4-triazol-5-yl)phenyl)imino)-λ⁶-sulfanone; ((4-(3-(difluoromethyl)-1H-1,2,4-triazol-5-yl)benzyl)imino)(methyl)(phenyl)-λ⁶-sulfanone; N-((4-methoxyphenyl)(methyl)(oxo)-λ⁶-sulfaneylidene)-4-(3-(trifluoromethyl)-1H-1,2,4-triazol-5-yl)benzamide; N-(2,4-difluorophenyl)-4-(3-(trifluoromethyl)-1H-1,2,4-triazol-5-yl)benzamide; N-(4-chloro-2-fluorophenyl)-4-(3-(trifluoromethyl)-1H-1,2,4-triazol-5-yl)benzamide; methyl(pyridin-3-yl)((4-(5-(trifluoromethyl)-1H-1,2,4-triazol-3-yl)benzyl)imino)-λ⁶-sulfanone; N-methyl-4-(5-(perfluoroethyl)-1,2,4-oxadiazol-3-yl)-N-(2-phenoxyethyl)benzamide; ethyl 4-(5-(trifluoromethyl)isoxazol-3-yl)benzoate; 4-(5-(trifluoromethyl)isoxazol-3-yl)benzoic acid; N-(2,4-difluorophenyl)-4-(5-(trifluoromethyl)isoxazol-3-yl)benzamide; N-((4-fluorophenyl)(methyl)(oxo)-λ⁶-sulfaneylidene)-4-(5-(trifluoromethyl)isoxazol-3-yl)benzamide; N-(methyl(oxo)(4-(trifluoromethyl)phenyl)-λ⁶-sulfaneylidene)-4-(5-(trifluoromethyl)isoxazol-3-yl)benzamide; N-((4-chlorophenyl)(methyl)(oxo)-λ⁶-sulfaneylidene)-4-(5-(trifluoromethyl)isoxazol-3-yl)benzamide; N-(methyl(oxo)(pyridin-4-yl)-λ⁶-sulfaneylidene)-4-(5-(trifluoromethyl)isoxazol-3-yl)benzamide; N-(methyl(oxo)(pyridin-2-yl)-λ⁶-sulfaneylidene)-4-(5-(trifluoromethyl)isoxazol-3-yl)benzamide; N-((4-methoxyphenyl)(methyl)(oxo)-λ⁶-sulfaneylidene)-4-(5-(trifluoromethyl)isoxazol-3-yl)benzamide; N-(4-chloro-2-fluorophenyl)-4-(2-(trifluoromethyl)oxazol-4-yl)benzamide; N-methyl-N-(2-phenoxyethyl)-4-(5-(trifluoromethyl)isoxazol-3-yl)benzamide; N-(methyl(oxo)(phenyl)-λ⁶-sulfaneylidene)-4-(5-(trifluoromethyl)isoxazol-3-yl)benzamide; N-methyl-N-(2-phenoxyethyl)-4-(2-(trifluoromethyl)oxazol-4-yl)benzamide; N-(methyl(oxo)(pyridin-4-yl)-λ⁶-sulfaneylidene)-4-(2-(trifluoromethyl)oxazol-4-yl)benzamide; N-((4-fluorophenyl)(methyl)(oxo)-λ⁶-sulfaneylidene)-4-(2-(trifluoromethyl)oxazol-4-yl)benzamide; 4-(5-chloro-2-(trifluoromethyl)oxazol-4-yl)-N-(2,4-difluorophenyl)benzamide; 4-(5-chloro-2-(trifluoromethyl)oxazol-4-yl)-N-methyl-N-(2-phenoxyethyl)benzamide; N-((4-chlorophenyl)(methyl)(oxo)-λ⁶-sulfaneylidene)-4-(2-(trifluoromethyl)oxazol-4-yl)benzamide; 4-(5-chloro-2-(trifluoromethyl)oxazol-4-yl)-N-((4-fluorophenyl)(methyl)(oxo)-λ⁶-sulfaneylidene)benzamide; 4-(5-chloro-2-(trifluoromethyl)oxazol-4-yl)-N-(methyl(oxo)(phenyl)-λ⁶-sulfaneylidene)benzamide; 4-(5-chloro-2-(trifluoromethyl)oxazol-4-yl)-N-((4-methoxyphenyl)(methyl)(oxo)-λ⁶-sulfaneylidene)benzamide; N-(2,4-difluorophenyl)-4-(2-(trifluoromethyl)oxazol-4-yl)benzamide; N-((4-methoxyphenyl)(methyl)(oxo)-λ⁶-sulfaneylidene)-4-(2-(trifluoromethyl)oxazol-4-yl)benzamide; methyl 4-(2-(trifluoromethyl)oxazol-4-yl)benzoate; N-(2,6-difluorophenyl)-4-(2-(trifluoromethyl)oxazol-4-yl)benzamide; N-phenyl-4-(2-(trifluoromethyl)oxazol-4-yl)benzamide; N-methyl-N-phenyl-4-(2-(trifluoromethyl)oxazol-4-yl)benzamide; N-(methyl(oxo)(phenyl)-λ⁶-sulfaneylidene)-4-(2-(trifluoromethyl)oxazol-4-yl)benzamide; N-((2-fluorophenyl)(methyl)(oxo)-λ⁶-sulfaneylidene)-4-(2-(trifluoromethyl)oxazol-4-yl)benzamide; methyl 4-(5-chloro-2-(trifluoromethyl)oxazol-4-yl)benzoate; 4-fluoro-N-(4-(5-(trifluoromethyl)-4,5-dihydro-1,2,4-oxadiazol-3-yl)benzyl)benzenesulfonamide; N-(4-(3-(trifluoromethyl)-1,2,4-oxadiazol-5-yl)benzyl)cyclobutanecarboxamide; 1-isopropyl-3-(4-(3-(trifluoromethyl)-1,2,4-oxadiazol-5-yl)benzyl)urea; 1-isopropyl-3-(4-(3-(trifluoromethyl)-1H-1,2,4-triazol-5-yl)benzyl)urea; 4-fluoro-N-(4-(3-(trifluoromethyl)-1H-1,2,4-triazol-5-yl)benzyl)benzenesulfonamide; 1-isopropyl-3-(4-(5-(trifluoromethyl)-4,5-dihydro-1,2,4-oxadiazol-3-yl)benzyl)urea; 4-fluoro-N-(4-(3-(trifluoromethyl)-1,2,4-oxadiazol-5-yl)benzyl)benzenesulfonamide; methyl(phenyl)((4-(3-(trifluoromethyl)-1,2,4-oxadiazol-5-yl)benzyl)imino)-λ⁶-sulfanone; (4-methoxyphenyl)(methyl)((4-(5-(trifluoromethyl)isoxazol-3-yl)phenyl)imino)-λ⁶-sulfanone; methyl(phenyl)((4-(5-(trifluoromethyl)isoxazol-3-yl)phenyl)imino)-λ⁶-sulfanone; tert-butyl (4-(5-(trifluoromethyl)isoxazol-3-yl)phenyl)carbamate; methyl(pyridin-2-yl)((4-(5-(trifluoromethyl)isoxazol-3-yl)phenyl)imino)-λ⁶-sulfanone; (2-fluorophenyl)(methyl)((4-(5-(trifluoromethyl)-1,3,4-oxadiazol-2-yl)phenyl)imino)-λ⁶-sulfanone; methyl(phenyl)((4-(5-(trifluoromethyl)-1,3,4-oxadiazol-2-yl)phenyl)imino)-λ⁶-sulfanone; (4-methoxyphenyl)(methyl)((4-(5-(trifluoromethyl)-1,3,4-oxadiazol-2-yl)phenyl)imino)-λ⁶-sulfanone; methyl(pyridin-2-yl)((4-(5-(trifluoromethyl)-1,3,4-oxadiazol-2-yl)phenyl)imino)-λ⁶-sulfanone; isopropyl(methyl)((4-(5-(trifluoromethyl)-1,3,4-oxadiazol-2-yl)phenyl)imino)-λ⁶-sulfanone; tert-butyl (4-(5-(trifluoromethyl)-1,3,4-oxadiazol-2-yl)phenyl)carbamate; N-(4-(5-(trifluoromethyl)-1,3,4-oxadiazol-2-yl)phenyl)cyclopropanecarboxamide; N-(methyl(oxo)(phenyl)-λ⁶-sulfaneylidene)-4-(5-(trifluoromethyl)-1,3,4-oxadiazol-2-yl)benzamide; N-((4-fluorophenyl)(methyl)(oxo)-λ⁶-sulfaneylidene)-4-(5-(trifluoromethyl)-1,3,4-oxadiazol-2-yl)benzamide; N-((4-methoxyphenyl)(methyl)(oxo)-λ⁶-sulfaneylidene)-4-(5-(trifluoromethyl)-1,3,4-oxadiazol-2-yl)benzamide; tert-butyl (4-(1-methyl-5-(trifluoromethyl)-1H-pyrazol-3-yl)phenyl)carbamate; tert-butyl (4-(1-methyl-3-(trifluoromethyl)-1H-pyrazol-5-yl)phenyl)carbamate; N-(4-(1-methyl-3-(trifluoromethyl)-1H-pyrazol-5-yl)phenyl)cyclopropanecarboxamide; 1-isopropyl-3-(4-(1-methyl-5-(trifluoromethyl)-1H-pyrazol-3-yl)phenyl)urea; N-ethyl-N-methyl-4-(3-(trifluoromethyl)-1,2,4-oxadiazol-5-yl)benzenesulfonamide; and N-ethyl-N-methyl-4-(3-(trifluoromethyl)-1H-1,2,4-triazol-5-yl)benzenesulfonamide.

The present invention also relates to a process for preparing the compound of Formula I. The compound of Formula I can be prepared by using any of the process steps as disclosed below, wherein the definition of the substituents is as described herein before if not mentioned otherwise:

Step 1:

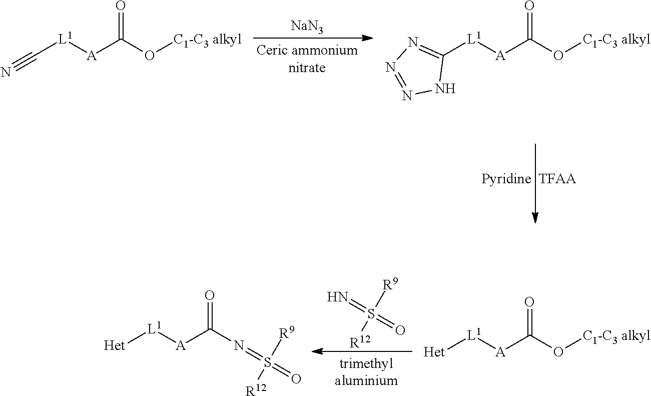

wherein, Het is

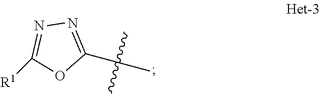

R¹ is CF₃; L¹ is a direct bond; and L² is

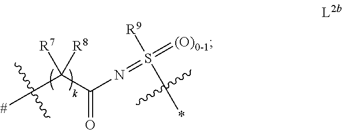

Step 2:

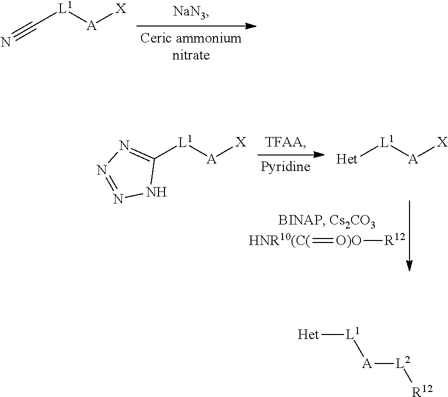

wherein, Het is

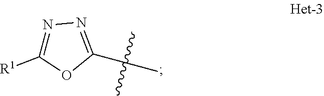

R¹ is CF₃; L¹ is a direct bond; L² is NR¹⁰(C(═O))O—; and X is Cl, Br or I;

step 3:

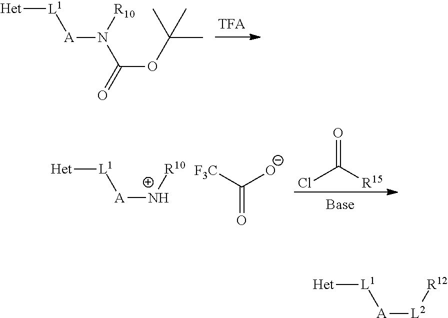

wherein, Het is

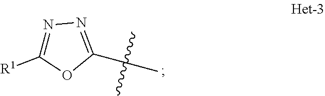

L¹ is a direct bond; and L² is —NR¹⁰;

step 4:

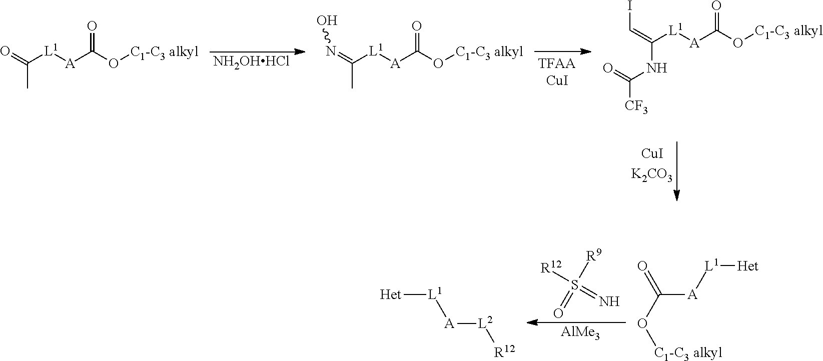

wherein, Het is

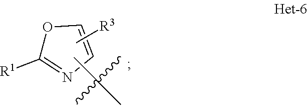

L¹ is a direct bond; and L² is

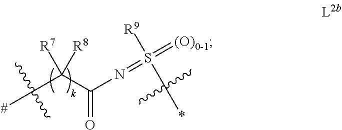

step 5:

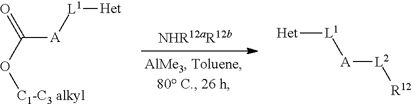

wherein, Het is

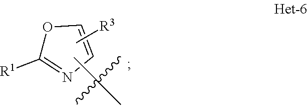

L¹ is a direct bond; and L² is —C(═O)—;

step 6:

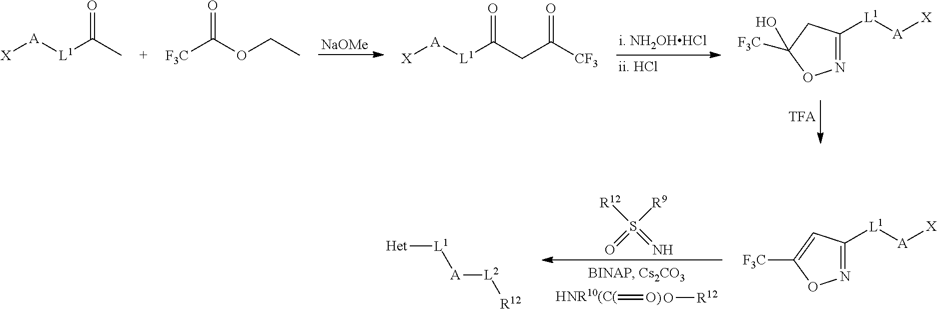

wherein, Het is

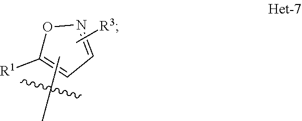

L¹ is a direct bond; L² is

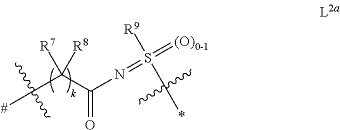

and X is Cl, Br or I;

step 7:

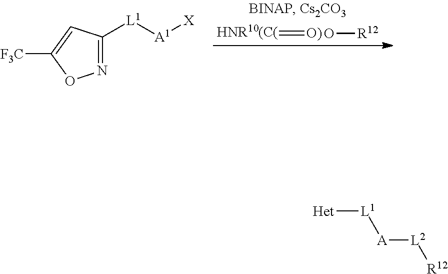

wherein, Het is

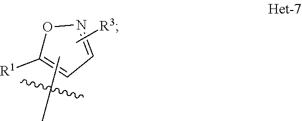

L¹ is direct bond; L² is NR¹⁰(C(═O))O—; and X is Cl, Br or I;

step 8:

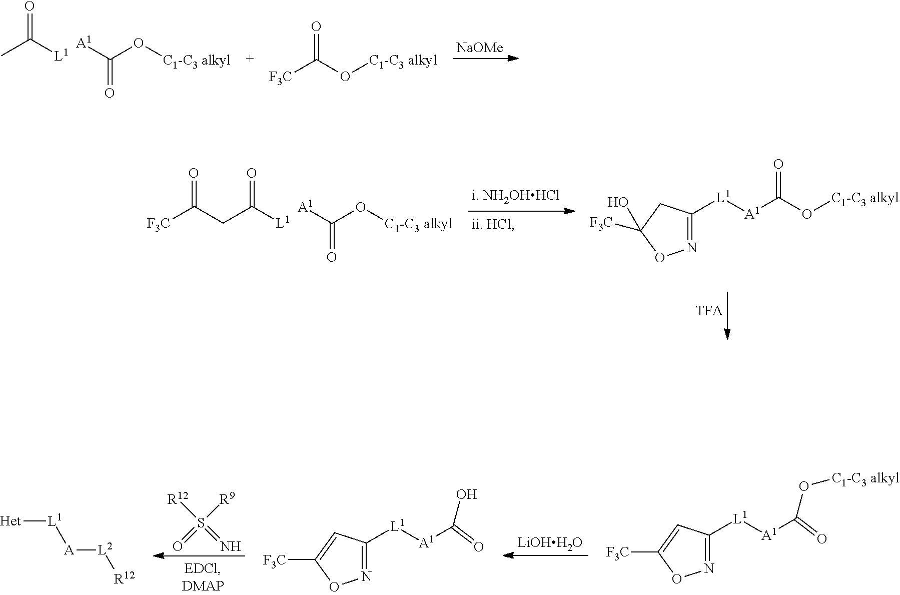

wherein, Het is

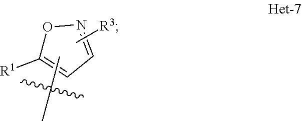

L¹ is a direct bond; and L² is

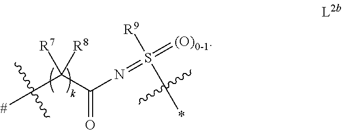

step 9:

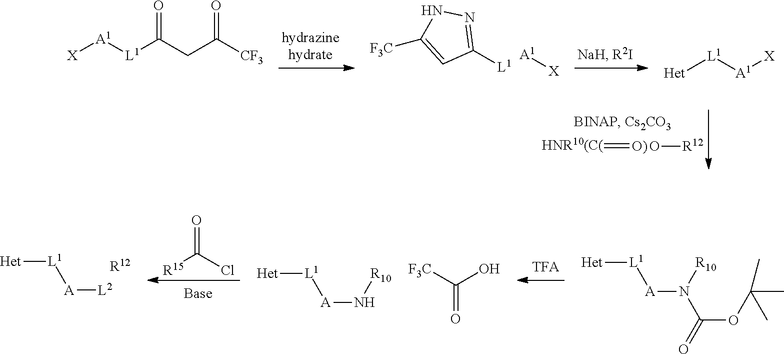

wherein, Het is

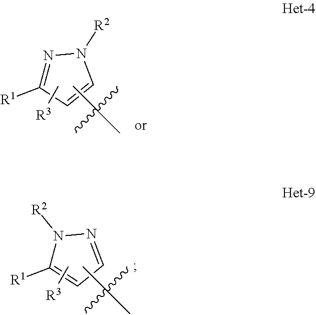

L¹ is a direct bond; L² is NR¹⁰(C(═O))O—; and X is Cl, Br or I;

step 10:

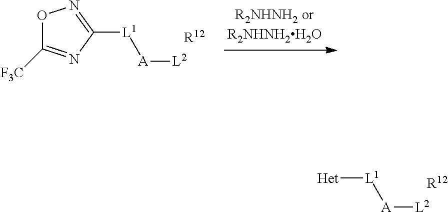

wherein, Het is

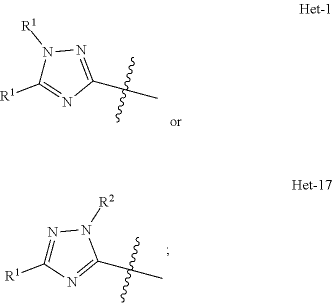

L¹ is a direct bond; and L² is —C(═O)—, —S(═O)₀₋₂—, —NR¹⁰—,

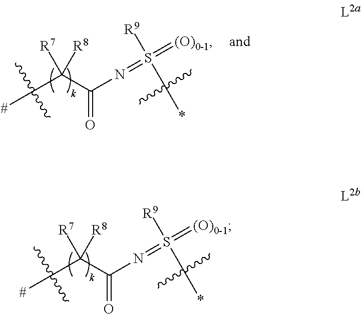

step 11:

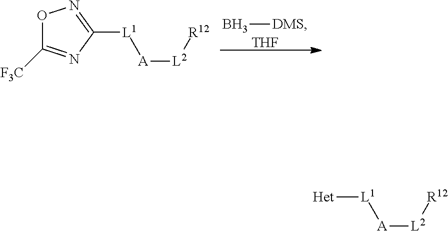

wherein, Het is

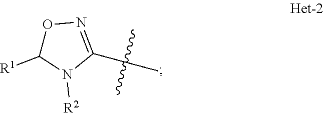

L¹ is a direct bond; and L² is —C(═O)—, —S(═O)₀₋₂—, —NR¹⁰—, or

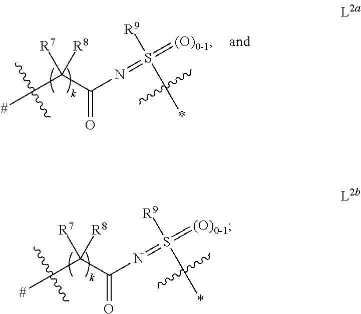

and

step 12:

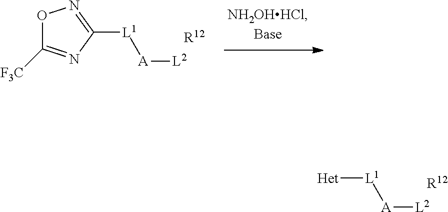

wherein, Het is

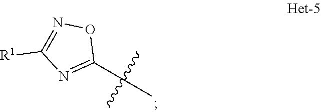

L¹ is a direct bond; and L² is —C(═O)—, —S(═O)₀₋₂—, —NR¹⁰—,

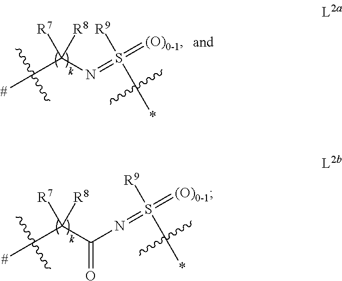

wherein, the definition of A, R¹, R², R³, R⁷, R⁸, R⁹, R¹⁰, R¹², R¹⁵, and k are as described herein before.

Compound/s of the present invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compound/s of the present invention may be present as a mixture of stereoisomers, individual stereoisomers or as an optically active form.

An anion part of the salt in case the compound/species of Formula 1 is a cation or capable of forming a cation can be inorganic or organic. Alternatively, a cation part of the salt in case the compound/species of Formula I is an anion or capable of forming anion can be inorganic or organic. Examples of the inorganic anion part of the salt include but are not limited to chloride, bromide, iodide, fluoride, sulfate, phosphate, nitrate, nitrite, hydrogen carbonates, and hydrogen sulfate. Examples of the organic anion part of the salt include but are not limited to formate, alkanoates, carbonates, acetates, trifluoroacetate, trichloroacetate, propionate, glycolate, thiocyanate, lactate, succinate, malate, citrates, benzoates, cinnamates, oxalates, alkylsulphates, alkylsulphonates, arylsulphonates aryldisulphonates, alkylphosphonates, arylphosphonates, aryldiphosphonates, p-toluenesulphonate, and salicylate. Examples of the inorganic cation part of the salt include but are not limited to alkali and alkaline earth metals. Examples of the organic cation part of the salt include but are not limited to protonated pyridine, methyl amine, imidazole, benzimidazole and histidine, to tetramethyl ammonium, tetrabutylammonium, to protonated choline and trimethylamine.

Metal ions in metal complexes of the compounds/species of Formula I are especially the ions of the elements of the second main group, especially calcium and magnesium, of the third and fourth main group, especially aluminium, tin and lead, and also of the first to eighth transition groups, especially chromium, manganese, iron, cobalt, nickel, copper, zinc and others. Particular preference is given to the metal ions of the elements of the fourth period and the first to eighth transition groups. Here, the metals can be present in the various valencies that they can assume.

Compound/s selected from Formula 1, (including all stereoisomers, N-oxides, and salts thereof), typically may exist in more than one form. Formula 1 thus includes all crystalline and non-crystalline forms of the compound/s that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula I can exhibit beneficial effects (e.g., suitability for preparation of useful formulations, improved biological performance) relative to another polymorph or a mixture of polymorphs of the same compound represented by Formula L Preparation and isolation of a particular polymorph of a compound represented by Formula I can be achieved by methods known to those skilled in the art including, for example, crystallization using selected solvents and temperatures.

In another embodiment the present invention relates to a composition comprising the compounds/species of Formula I agriculturally acceptable salts, metal complexes, constitutional isomers, stereo-isomers, diastereoisomers, enantiomers, chiral isomers, atropisomers, conformers, rotamers, tautomers, optical isomers, polymorphs, geometric isomers, or N-oxides thereof optionally with one or more additional active ingredient with the auxiliary such as inert carrier or any other essential ingredient such as surfactants, additives, solid diluents and liquid diluents.

The compound/species of Formula I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compound/species of Formula I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants. Particularly, the compound/species of Formula I and the compositions according to the invention are important in the control of phytopathogenic fungi on soybeans and on the plant propagation material, such as seeds, and the crop material of soybeans. Accordingly, the present invention also includes a composition comprising at least one compound of Formula I and seed. The amount of the compound/s of Formula I in the composition ranges from 0.1 gai (gram of active ingredient) to 10 kgai (kilogram of active ingredient) per 100 kg of seeds.

Preferably, compound/species of Formula I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative or reproductive parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts, twigs, flowers, and other parts of plants, including seedlings and young plants, which are to be transplanted after germination or after emergence from soil.

These young plants may also be protected before transplantation by a total or partial treatment by immersion or pouring.

Preferably, treatment of plant propagation materials with compound/s of Formula I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties. Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1 185; and references quoted therein. Several cultivated plants have been rendered tolerant to herbicides by conventional methods of breeding (mutagenesis), e. g. Clearfield© summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as 6-endotoxins, e. g. CrylA(b), CrylA(c), CrylF, CrylF(a2), CryllA(b), CrylllA, CrylllB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO02/015701). Further examples of such toxins or genetically modified plants capable of synthesizing such toxins are disclosed, e. g., in EP374753, WO93/007278, WO95/34656, EP427 529, EP451 878, WO03/18810 und WO03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the CrylAb toxin), YieldGard© Plus (corn cultivars producing CrylAb and Cry3Bb1 toxins), Starlink® (corn cultivars producing the Cry9c toxin), Herculex® RW (corn cultivars producing Cry34Abl, Cry35Ab1 and the enzyme phosphinothricin-N-acetyltransferase [PAT]); NuCOTN® 33B (cotton cultivars producing the CrylAc toxin), Bollgard® I (cotton cultivars producing the Cry1 Ac toxin), Bollgard® II (cotton cultivars producing CrylAc and Cry2Ab2 toxins); VIPCOT® (cotton cultivars producing a VIP-toxin); NewLeaf® (potato cultivars producing the Cry3A toxin); Bt-Xtra, NatureGard, KnockOut®, BiteGard®, Protecta®, Bt1 1 (e. g. Agrisure® CB) and Bt176 from Syngenta Seeds SAS, France, (corn cultivars producing the CrylAb toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the CrylAc toxin) and 1507 from Pioneer Overseas Corporation, Belgium (corn cultivars producing the Cry1 F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP392225), plant disease resistance genes (e. g. potato cultivars, which express resistance genes acting against Phytophthora infestans derived from the Mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The present invention also relates to a method for controlling or preventing infestation of plants by phytopathogenic micro-organisms in agricultural crops and or horticultural crops wherein an effective amount of at least one compound of formula I or the combination of the present invention or the composition of the present invention, is applied to the seeds of plants. The compound/s, combination/s and composition/s of the present invention can be used for controlling or preventing plant diseases. Particularly, the compound/s of Formula I and compositions thereof, respectively, are particularly suitable for controlling the following plant diseases:

Albugo spp. (white rust) on ornamentals, vegetables (e. g. A. Candida) and sunflowers (e. g. A. tragopogonis); Altemaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (6. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (£. pyri), soft fruits (£. veneta: anthracnose) and vines (£. ampelina: anthracnose); Entyloma oryzae (leaf smut) on rice; Epicoccum spp. (black mold) on wheat; Erysiphe spp. (powdery mildew) on sugar beets (£. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), soybeans, potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compound of formula I, the combinations or the compositions thereof may be used to treat several fungal pathogens. Non-limiting examples of pathogens of fungal diseases which can be treated in accordance with the invention include:

Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries. Blastocladiomycetes, such as Physoderma maydis. Mucoromycetes, such as Choanephora cucurbitarum.; Mucor spp.; and Rhizopus arrhizus,

In another embodiment diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis order Puccinia striiformis; Uromyces species, for example Uromyces appendiculatus.

In particular, Cronartium ribicola (White pine blister rust); Gymnosporangium juniperi-virginianae (Cedar-apple rust); Hemileia vastatrix (Coffee rust); Phakopsora meibomiae and P. pachyrhizi (Soybean rust); Puccinia coronata (Crown Rust of Oats and Ryegrass); Puccinia graminis (Stem rust of wheat and Kentucky bluegrass, or black rust of cereals); Puccinia hemerocallidis (Daylily rust); Puccinia persistens subsp. triticina (wheat rust or ‘brown or red rust’); Puccinia sorghi (rust in corn); Puccinia striiformis (‘Yellow rust’ in cereals); Uromyces appendiculatus (rust of beans); Uromyces phaseoli (Bean rust); Puccinia melanocephala (‘Brown rust’ in sugarcane); Puccinia kuehnii (‘Orange rust’ in sugarcane).

Plants which can be treated in accordance with the invention include the following: cotton, flax, grapevine, fruits, vegetables, such as Rosaceae sp (for example pome fruits such as apples, pears, apricots, cherries, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Vitaceae sp. (for example grapes); Solanaceae sp. (for example tomatoes, peppers), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Poaceae/Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); Malvaceae (for example cotton); useful plants and ornamental plants for gardens and wooded areas; and genetically modified varieties of each of these plants.

More preference is given to controlling the following diseases of soya beans: Fungal diseases on leaves, stems, pods and seeds caused, for example, by Altemaria leaf spot (Altemaria spec. Atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectiia crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidennatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thiclaviopsis root rot (Thielaviopsis basicola).

The present invention also relates to the use of compound of formula I, the combinations or the compositions thereof for controlling or preventing the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye and Phakopsoraceae spp. on various plants, in particular Phakopsora pachyrhizi and P. meibomiae (soybean rust) on soybeans, Hemileia vastatrix (Coffee rust), Uromyces appendiculatus, Uromyces fabae and Uromyces phaseoli (rust of beans).

The present invention further relates to the use of compound of formula I, the combinations or the compositions thereof for controlling or preventing against phytopathogenic fungi such as Phakopsora pachyrhizi, Phakopsora meibomiae, of agricultural crops and or horticultural crops.

The compound/s of Formula I, the combinations and the compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria.

As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Por/a spp., Serpula spp. and Tyromyces spp., Deuteromycetes such as Aspergillus spp., Cladosporium spp., Penicillium spp., Trichoderma spp., Altemaria spp., Paecilomyces spp. and Zygomycetes such as Mucor spp., and in addition in the protection of stored products and harvest the following yeast fungi are worthy of note: Candida spp. and Saccharomyces cerevisae.

In one embodiment the compound/s of Formula I, the combinations and the compositions thereof, respectively, are particularly suitable for controlling the following plant diseases: Phakopsora pachyrhizi and P. meibomiae (soybean rust) on soybeans.

The present invention further relates to a method for controlling or preventing phytopathogenic fungi. The method comprises treating the fungi or the materials, plants, plant parts, locus thereof, soil or seeds to be protected against fungal attack, with an effective amount of at least one compound of Formula I or the combinations or the compositions comprising at least one compound of Formula I.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compound/s of Formula I, the combinations and the compositions thereof, respectively, may be used for improving the health of a plant. The invention also relates to a method for improving plant health by treating a plant, its propagation material and/or the locus where the plant is growing or is to grow with an effective amount of compound/s I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compound/s of Formula I can be present in different crystal modifications or polymorphs whose biological activity may differ. They are likewise subject matter of the present invention.

The compound/s of Formula I are employed as such or in the form of compositions for treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compound/s of Formula I, the combinations and the compositions thereof protectively either at or before planting or transplanting.

The invention also relates to agrochemical compositions comprising an auxiliary and at least one compound of Formula I.

An agrochemical composition comprises a fungicidally effective amount of a compound of Formula I. The term “effective amount” denotes an amount of the composition or of the compound/s of Formula I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound of Formula I used.

The compound/s of Formula I, their -oxides, metal complexes, isomers, polymorphs or the agriculturally acceptable salts thereof can be converted into customary types of agrochemical compositions, c. g. solutions, emulsions, suspensions, dusts, powders, pastes, granules, pressings, capsules, and mixtures thereof. Examples for composition types are suspensions (c. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubcmann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof. Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide.

Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound of Formula I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound of Formula I and 5-15 wt % wetting agent (e. g. alcohol alkoxylates) are dissolved in water and/or in a water-soluble solvent (e. g. alcohols) ad 100 wt %. The active substance dissolves upon dilution with water.

### ii) Dispersible Concentrates (DC)

5-25 wt % of a compound of Formula I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound of Formula I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

### iv) Emulsions (EW, EO, ES)

5-40 wt % of a compound of Formula I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound of Formula I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

### vi) Water-Dispersible Granules and Water-Soluble Granules (WG, SG)

50-80 wt % of a compound of Formula I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance. vii) Water-dispersible powders and water-soluble powders (WP, SP, WS) 50-80 wt % of a compound of Formula I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound of Formula I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

### ix) Microemulsion (ME)

5-20 wt % of a compound of Formula I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

### x) Microcapsules (CS)

An oil phase comprising 5-50 wt % of a compound of Formula I, 0-40 wt % water insoluble organic solvent

(e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound of Formula I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

### xi) Dustable Powders (DP, DS)

1-10 wt % of a compound of Formula I are ground finely and mixed intimately with solid carrier (e. g. finely divided kaolin) ad 100 wt %.

xii) Granules (GR, FG)

0.5-30 wt % of a compound of Formula I are ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound of Formula I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active ingredient (ai). The active ingredients (ai) are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations.

Application can be carried out before or during sowing. Methods for applying compound/s of Formula I, the combinations and the compositions thereof, respectively, onto plant propagation material, especially seeds, include dressing, coating, pelleting, dusting, and soaking as well as in-furrow application methods. Preferably, compound/s of Formula I, the combinations and the compositions thereof, respectively, are applied on to the plant propagation material by a method such that germination is not induced, e. g. by seed dressing, pelleting, coating and dusting.

When employed in plant protection, the amounts of active substances applied arc, depending on the kind of effect desired, from 0.001 to 2 kg per ha, preferably from 0.005 to 2 kg per ha, more preferably from 0.05 to 1.0 kg per ha, and in particular from 0.1 to 1.0 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:20 to 20:1.

A pesticide is generally a chemical or biological agent (such as pesticidally active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

The compound/s of Formula I, the combinations and the compositions thereof comprising them in the use as fungicides with other fungicides may result in an expansion of the fungicidal spectrum of activity being obtained or in a prevention of fungicide resistance development. Furthermore, in many cases, extraordinary effects are obtained.

The present invention also relates to the combination comprising at least one compound of Formula I and at least one further pesticidally active substance selected from the group of fungicides, insecticides, nematicides, acaricides, biopesticides, herbicides, safeners, plant growth regulators, antibiotics, fertilizers and nutrients. The pesticidally active substances reported in WO2015185485 pages 36-43 and WO2017093019 pages 42-56 can be used in conjunction with which the compound/s of Formula I.

The active substances referred to as component 2, their preparation and their activity e. g. against harmful fungi is known (cf.: http://www.alanwood.net/pesticides/); these substances are commercially available. The compounds described by IU PAC nomenclature, their preparation and their pesticidal activity are also known (cf. Can. J. Plant Sci. 48(6), 587-94, 1968; EP141317; EP152031; EP226917; EP243970; EP256503; EP428941; EP532022; EP1028125; EP1035122; EP1201648; EP1122244, JP2002316902; DE19650197; DE10021412; DE102005009458; U.S. Pat. Nos. 3,296,272; 3,325,503; WO9846608; WO9914187; WO9924413; WO9927783; WO0029404; WO0046148; WO0065913; WO0154501; WO 0156358; WO0222583; WO0240431; WO0310149; WO0311853; WO0314103; WO0316286; WO0353145; WO0361388; WO0366609; WO0374491; WO0449804; WO0483193; WO05120234; WO05123689; WO05123690; WO0563721; WO0587772; WO0587773; WO0615866; WO0687325; WO0687343; WO0782098; WO0790624; WO11028657; WO2012168188; WO2007006670; WO201177514; WO13047749; WO10069882; WO13047441; WO0316303; WO0990181; WO13007767; WO1310862; WO13127704; WO13024009; WO13024010; WO13047441; WO13162072; WO13092224 and WO11135833.

The present invention furthermore relates to agrochemical mixtures comprising at least one compound of Formula I (component 1) and at least one further active substance useful for plant protection, e. g. selected from the groups A) to 0) (component 2), in particular one further fungicide, e. g. one or more fungicide from the groups A) to K), as described above, and if desired one suitable solvent or solid carrier. Furthermore, combating harmful fungi with a mixture of compound/s of Formula I and at least one fungicide from groups A) to K), as described above, is advantageous than combating those fungi with individual compound/s of Formula I or individual fungicides from groups A) to K).

By applying compound/s of Formula I together with at least one pesticidally active substance from groups A) to O) an enhanced effect can be obtained.

This can be obtained by applying the compound/s of Formula I and at least one further pesticidally active substance simultaneously, either jointly (e. g. as tank-mix) or separately, or in succession, wherein the time interval between the individual applications is selected to ensure that the active substance applied first still occurs at the site of action in a sufficient amount at the time of application of the further pesticidally active substance(s). The order of application is not essential for working of the present invention.

When applying compound/s of Formula I and a pesticidally active substance sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In the binary mixtures and compositions according to the invention the weight ratio of the component 1) and the component 2) generally depends from the properties of the active components used, usually it is in the range of 1:100 to 100:1, regularly in the range of 1:50 to 50:1, preferably in the range of 1:20 to 20:1, more preferably in the range of 1:10 to 10:1, even more preferably in the range of 1:4 to 4:1 and in particular in the range of 1:2 to 2:1.

According to a further embodiment of the binary mixtures and compositions thereof, the weight ratio of the component 1) and the component 2) usually is in the range of 1000:1 to 1:1000, often in the range of 100:1 to 1:100, regularly in the range of 50:1 to 1:50, preferably in the range of 20:1 to 1:20, more preferably in the range of 10:1 to 1:10, even more preferably in the range of 4:1 to 1:4 and in particular in the range of 2:1 to 1:2.

In the ternary mixtures, i.e. compositions according to the invention comprising the component 1) and component 2) and a compound III (component 3), the weight ratio of component 1) and component 2) depends from the properties of the active substances used, usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1, and the weight ratio of component 1) and component 3) usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1.

Any further active components are, if desired, added in a ratio of 20:1 to 1:20 to the component 1).

These ratios are also suitable for inventive mixtures applied by seed treatment.

The present invention also relates to a process for preparing the compound/s of the present invention. The process for preparing the compound/s of the present invention is described in the experimental section in more detail.

The invention disclosed in the present disclosure shall now be elaborated with the help of non-limiting schemes and examples.

### Chemistry Schemes:

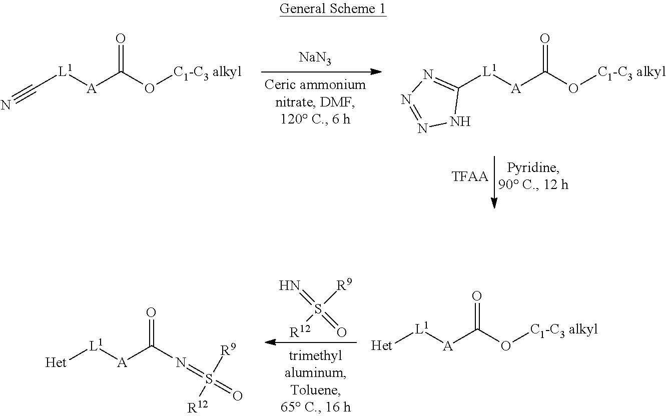 [0226] wherein, Het is

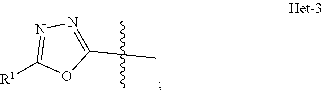

R¹ is CF₃; L¹ is a direct bond; and L² is

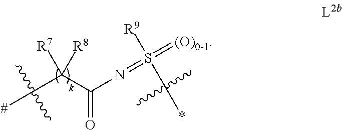

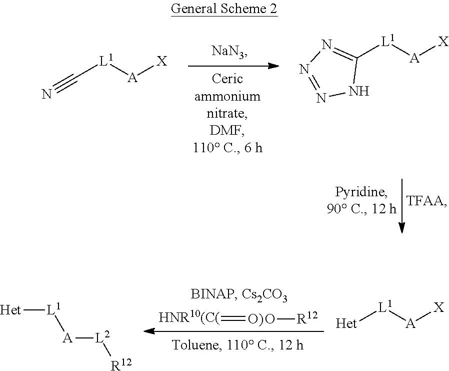

wherein, Het is

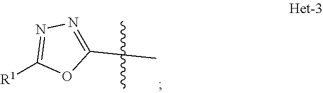

R¹ is CF₃; L¹ is a direct bond; L² is NR¹⁰(C(═O))O—; and X is Cl, Br or I.

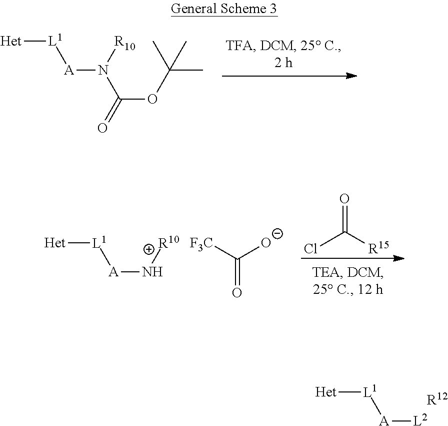

wherein, Het is

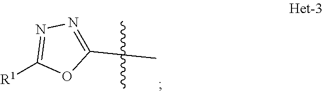

L¹ is a direct bond; and L² is —NR¹⁰.

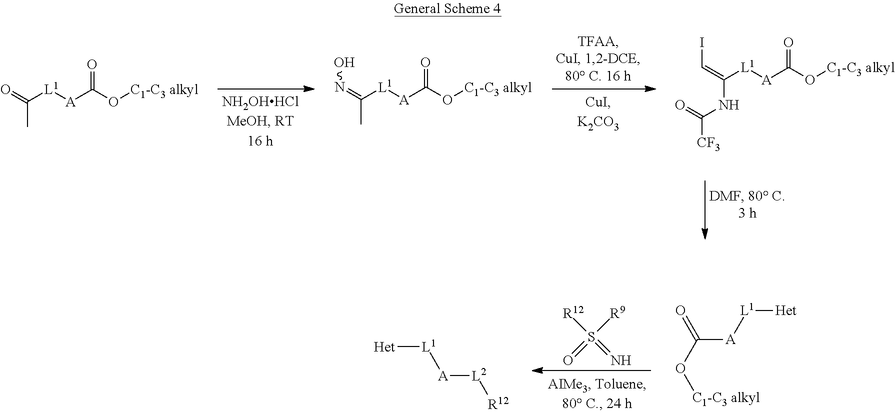

wherein, Het is

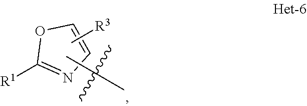

L¹ is a direct bond and L² is

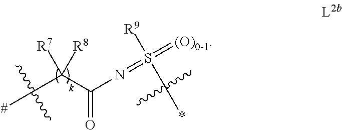

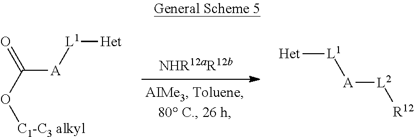

Het is

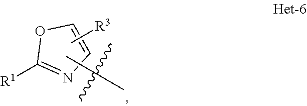

L¹ is a direct bond; and L² is C(═O)—.

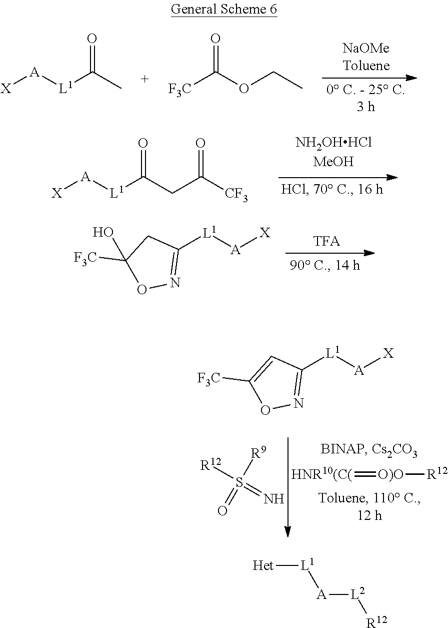

wherein, Het is

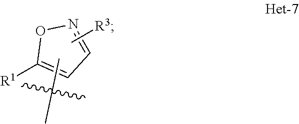

L¹ is a direct bond; L² is

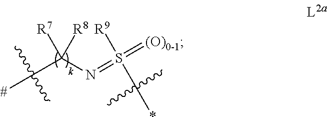

and X is Cl, Br or I.

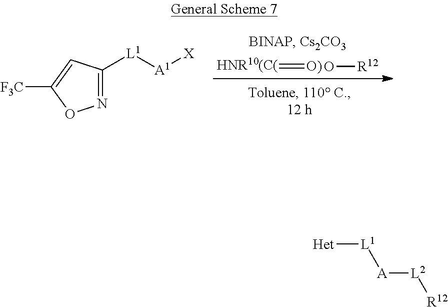

wherein, Het is

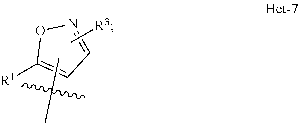

L¹ is direct bond; L² is NR¹⁰(C(═O))O—; and X is Cl, Br or I.

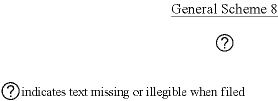

wherein, Het is

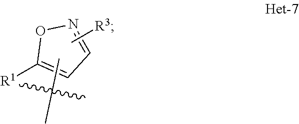

L¹ is a direct bond; and L² is

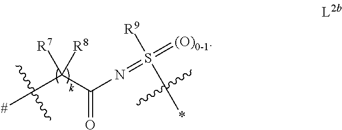

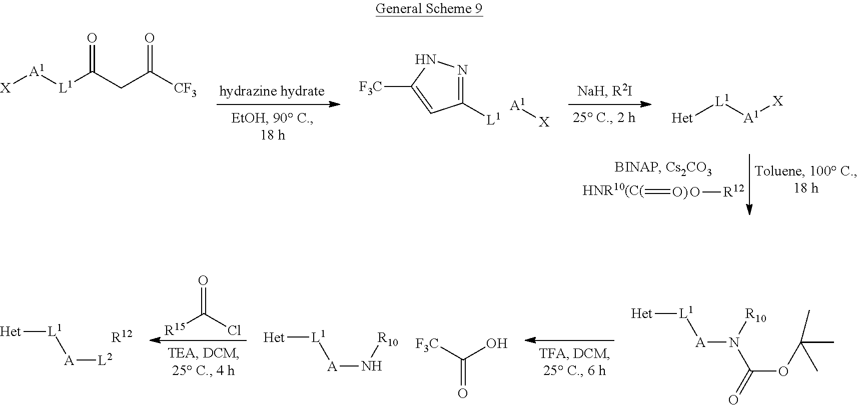

wherein, Het is

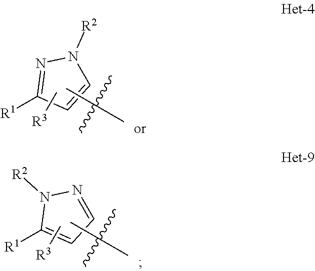

L¹ is a direct bond; L² is NR¹⁰(C(═O))O—; and X is Cl, Br or I.

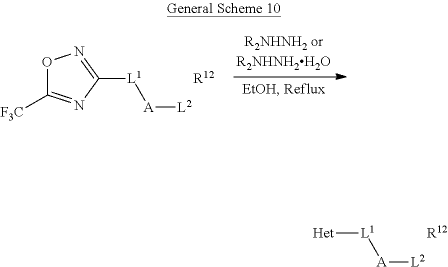

wherein, Het is

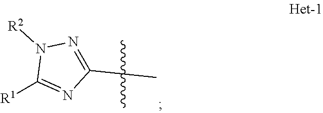

L¹ is a direct bond; and L² is —C(═O)—, —S(═O)₀₋₂—, —NR¹⁰—,

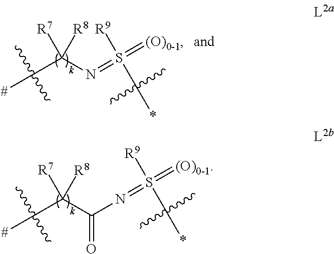

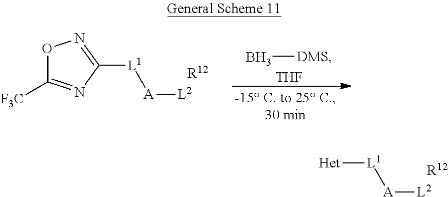

wherein, Het is

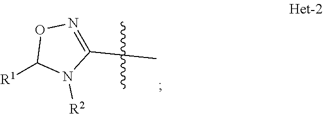

L¹ is a direct bond; and L² is —C(═O)—, —S(═O)₀₋₂—, —NR¹⁰—,

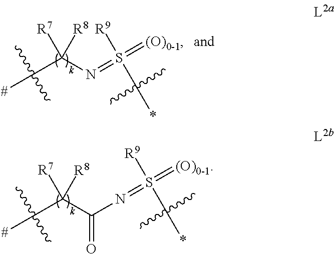

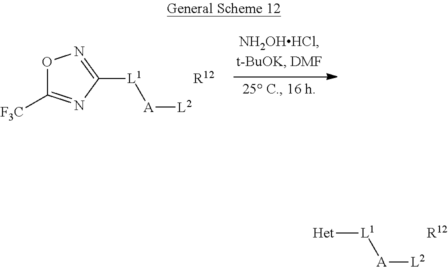

wherein, Het is

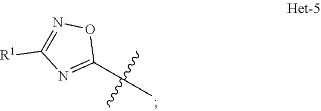

L¹ is a direct bond; and L² is —C(═O)—, —S(═O)₀₋₂—, —NR¹⁰—,

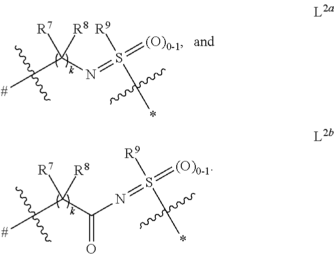

The definitions as described in the description shall apply for the substituents if not mentioned otherwise in the general schemes 1 to 12.

### CHEMISTRY EXAMPLES

### Example 1:—Preparation of N-((4-chlorophenyl)(methyl)(oxo)-λ⁶-sulfaneylidene)-4-(5-(trifluoromethyl)-4,5-dihydro-1,2,4-oxadiazol-3-yl)benzamide (Compound 1)

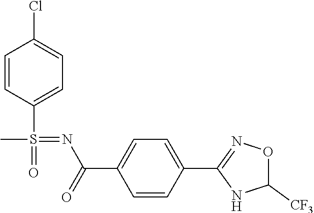 [SMILES: CS(=O)(=NC(=O)c1ccc(C2=NOC(C(F)(F)F)N2)cc1)c1ccc(Cl)cc1]

### Step-1: Methyl 4-cyanobenzoate

To a stirred solution of 4-cyanobenzoic acid (25 g, 170 mmol) in N,N-dimethylformamide (120 mL), potassium carbonate (35.2 g, 255 mmol) was added at 25° C. The reaction mixture was cooled to 0-5° C. and methyl iodide (15.9 mL, 255 mmol) was added slowly. The reaction mixture was stirred at 45° C. for 2 h. After completion of the reaction, the reaction mixture was cooled to 25° C. and crushed ice was poured into it with stirring. The precipitate obtained was filtered and dried under reduced pressure to obtain methyl 4-cyanobenzoate (24.6 g, 153 mmol, 90% yield).

### Step-2: Methyl-4-(N′-hydroxycarbamimidoyl)benzoate

To a stirred solution of methyl 4-cyanobenzoate (0.9 g, 5.6 mmol) in methanol (12 mL), sodium bicarbonate (0.52 g, 6.1 mmol) and hydroxylamine hydrochloride (0.4 g, 5.6 mmol) were added. The reaction mixture was heated to reflux at 75° C. for 4 h. After completion of the reaction, the reaction mixture was cooled to 25° C. and filtered. The filtrate was concentrated under reduced pressure to obtain methyl 4-(N′-hydroxycarbamimidoyl)benzoate (0.9 g, 4.6 mmol, 83% yield).

### Step-3: Methyl 4-(5-(trifluoromethyl)-1,2,4-oxadiazol-3-yl)benzoate

To a stirred solution of methyl 4-(N′-hydroxycarbamimidoyl)benzoate (0.9 g, 4.6 mmol) in tetrahydrofuran (10 mL), trifluroro acetic acid (1 mL, 6.9 mmol) was added at 0-5° C. under nitrogen atmosphere. The reaction mixture was stirred at 25° C. for 16 h. The reaction mixture was diluted with ethyl acetate (30 mL). The ethyl acetate layer was washed three times with a saturated solution of sodium bicarbonate (30 mL), dried over anhydrous sodium sulphate and concentrated under reduced pressure. The crude residue was purified by column chromatography using eluent 0-30% ethyl acetate in hexane on silica gel to obtain methyl 4-(5-(trifluoromethyl)-1,2,4-oxadiazol-3-yl)benzoate (0.55 g, 2. mmol, 43.6% yield).

### Step 4: 4-(5-(Trifluoromethyl)-1,2,4-oxadiazol-3-yl)benzoic Acid

To a stirred solution of methyl 4-(5-(trifluoromethyl)-1,2,4-oxadiazol-3-yl)benzoate (15 g, 55.1 mmol) in tetrahydrofuran (120 mL), a solution of sodium hydroxide (4 g, 99 mmol) in water (30 mL) was added drop wise at 0-5° C. The reaction mixture was stirred for 16 h at 25° C. After completion of the reaction, tetrahydrofuran was removed by evaporation under reduced pressure. Water (50 mL) was added to the residue and cooled to 0-5° C. The mixture was acidified to pH 3 with 3N aqueous HCl. The precipitate was collected by filtration, washed with water, and dried to obtain 4-(5-(trifluoromethyl)-1,2,4-oxadiazol-3-yl)benzoic acid (11.2 g, 43.4 mmol, 79% yield).

### Step-5: (2-Fluorophenyl)(methyl)sulfane

To a stirred solution of 4-chlorobenzenethiol (2.6 g, 17.9 mmol) and acetonitrile (25 mL), potassium carbonate (6.21 g, 44.9 mmol) was added at 25° C. and cooled to 0-5° C. Then methyl iodide (1.2 mL, 19.8 mmol) was added drop wise. The reaction mixture was then stirred at 25° C. for 3 h. After completion of the reaction, it was diluted with dichloromethane (40 mL) and washed thrice with water (15 mL). The dichloromethane layer was separated, dried over anhydrous sodium sulphate and concentrated under reduced pressure to obtain (4-chlorophenyl)(methyl)sulfane (2.96 g).

### Step-6: (4-Chlorophenyl)(imino)(methyl)-λ⁶-sulfanone

To a stirred solution of (4-chlorophenyl)(methyl)sulfane (2.9 g, 18.3 mmol) in methanol (10 mL) and acetonitrile (25 mL), ammonium carbamate (3.1 g, 40 mmol) was added and the reaction mixture was cooled to 0-5° C. Then iodobenzene diacetate (12.9 g, 40 mmol) was added portion wise in 3 minutes at 0-5° C. This mixture was stirred at 25° C. for 16 h. The reaction mixture was concentrated under reduced pressure. The residue was purified by column chromatography on silica gel using eluent hexane to 80% ethyl acetate in hexane to obtain (4-chlorophenyl)(imino)(methyl)-λ⁶-sulfanone (1.6 g, 46% yield).

### Step-7:—N-((4-chlorophenyl)(methyl)(oxo)-λ⁶-sulfanylidene)-4-(5-(trifluoromethyl)-1,2,4-oxadiazol-3-yl)benzamide

To a stirred solution of 4-(5-(trifluoromethyl)-1,2,4-oxadiazol-3-yl)benzoic acid (0.8 g, 3.1 mmol) in dichloromethane (10 mL), 1-(3-dimethylaminopropyl)-3-ethylcarbodiimide hydrochloride (1.2 g, 6.2 mmol) and 4-dimethylaminopyridine (1.136 g, 9.30 mmol) were added at 5-10° C. under nitrogen atmosphere. Then (4-chlorophenyl)(imino)(methyl)-λ⁶-sulfanone (0.9 g, 4.6 mmol) was added and stirred at 25° C. for 16 h. The crude product was extracted with dichloromethane (30 mL). The dichloromethane layer was washed two times with brine solution (10 mL), dried over anhydrous sodium sulphate and concentrated. The residue was purified by column chromatography on silica gel using eluent hexane to 50% ethyl acetate in hexane. Pure fractions were combined and concentrated to obtain N-((4-chlorophenyl)(methyl)(oxo)-λ⁶-sulfanylidene)-4-(5-(trifluoromethyl)-1,2,4-oxadiazol-3-yl)benzamide (0.7 g, 1.6 mmol, 52.6% yield).

### Step 8: N-((4-chlorophenyl)(methyl)(oxo)-λ⁶-sulfaneylidene)-4-(5-(trifluoromethyl)-4,5-dihydro-1,2,4-oxadiazol-3-yl)benzamide (Compound 1)

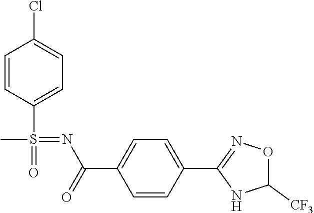 [SMILES: CS(=O)(=NC(=O)c1ccc(C2=NOC(C(F)(F)F)N2)cc1)c1ccc(Cl)cc1]

To a stirred solution of N-((4-chlorophenyl)(methyl)(oxo)-λ⁶-sulfanylidene)-4-(5-(trifluoromethyl)-1,2,4-oxadiazol-3-yl)benzamide (0.1 g, 0.2 mmol) and tetrahydrofuran (3 mL), borane-dimethyl sulfide complex (0.06 mL, 0.7 mmol) was added at −15° C. to −10° C.° and stirred at 0° C. for 15 minutes. The reaction was quenched by drop wise addition of methanol (5 mL). The reaction mixture was concentrated and the residue obtained was purified by column chromatography using eluent hexane to 50% ethyl acetate in hexane to obtain N-((4-chlorophenyl)(methyl)(oxo)-λ⁶-sulfaneylidene)-4-(5-(trifluoromethyl)-4,5-dihydro-1,2,4-oxadiazol-3-yl)benzamide (0.062 g, 61.7%).

TABLE 1
The following compounds were prepared by the process analogous to that of the
compound 1.

| Compound | | | |
|---|---|---|---|
| no. | Structure | Iupac name | Yield |
| 6 | 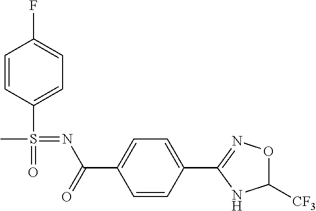 [SMILES: CS(=O)(=NC(=O)c1ccc(C2=NOC(C(F)(F)F)N2)cc1)c1ccc(F)cc1] | N-((4- fluorophenyl)(methyl)(oxo)-λ⁶- sulfaneylidene)-4-(5- (trifluoromethyl)-4,5-dihydro- 1,2,4-oxadiazol-3-yl)benzamide | 184 mg, yield- 92% |
| 10 | 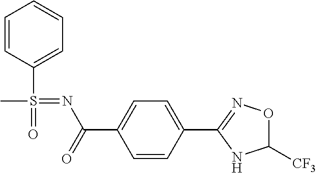 [SMILES: CS(=O)(=NC(=O)c1ccc(C2=NOC(C(F)(F)F)N2)cc1)c1ccccc1] | N-(methyl(oxo)(phenyl)-λ⁶- sulfanylidene)-4-(5- (trifluoromethyl)-4,5-dihydro- 1,2,4-oxadiazol-3-yl)benzamide | 243 mg, yield- 60% |
| 25 | 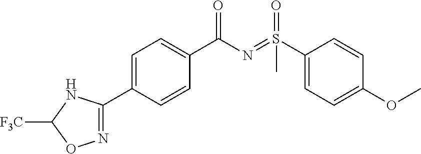 [SMILES: COc1ccc(S(C)(=O)=NC(=O)c2ccc(C3=NOC(C(F)(F)F)N3)cc2)cc1] | N-((4- methoxyphenyl)(methyl)(oxo)- λ⁶-sulfaneylidene)-4-(5- (trifluoromethyl)-4,5-dihydro- 1,2,4-oxadiazol-3-yl)benzamide | 0.27 g, 42% yield |

### Example 2: Preparation of N-((4-fluorophenyl)(methyl)(oxo)-λ⁶-sulfaneylidene)-4-(5-(trifluoromethyl)-1h-1,2,4-triazol-3-yl)benzamide

To a stirred solution of N-((4-fluorophenyl)(methyl)(oxo)-λ⁶-sulfanylidene)-4-(5-(trifluoromethyl)-1,2,4-oxadiazol-3-yl)benzamide (0.5 g, 1.2 mmol) in ethanol (12 mL), hydrazine hydrate (0.1 mL, 1.8 mmol) was added at 25° C. The reaction mixture was stirred at 40° C. for 12 h. The reaction mixture was evaporated under reduced pressure. The crude residue was purified using column chromatography using eluent-hexane to 50% ethyl acetate in hexane on silica gel to obtain N-((4-fluorophenyl)(methyl)(oxo)-λ⁶-sulfanylidene)-4-(5-(trifluoromethyl)-1h-1,2,4-triazol-3-yl)benzamide (138 mg, 0.3 mmol, 27.7% yield).

TABLE 2
The following compounds were prepared by the process analogous to that of the
compound 7.

| Compound | | | |
|---|---|---|---|
| no. | Structure | Iupac name | Yield |
| 8 | 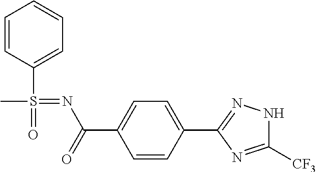 [SMILES: CS(=O)(=NC(=O)c1ccc(-c2n[nH]c(C(F)(F)F)n2)cc1)c1ccccc1] | N-(methyl(oxo)(phenyl)-λ⁶- sulfaneylidene)-4-(5- (trifluoromethyl)-1h-1,2,4-triazol-3- yl)benzamide | 121 mg, 24.26% yield |
| 43 | 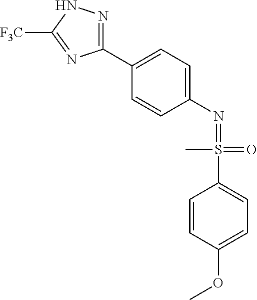 [SMILES: COc1ccc(S(C)(=O)=Nc2ccc(-c3n[nH]c(C(F)(F)F)n3)cc2)cc1] | (4-methyoxyphenyl)(methyl)((4-(5- (trifluoromethyl)-1h-1,2,4-triazol-3- yl)phenyl)imino)-λ⁶-sulfanone | 0.13 g, 22.39% yield |

### Example 3:—Preparation of N-methyl-N-(2-phenoxyethyl)-4-(5-(trifluoromethyl)-1h-1,2,4-triazol-3-yl)benzamide (Compound 11)

To a stirred solution of N-methyl-N-(2-phenoxyethyl)-4-(5-(trifluoromethyl)-1,2,4-oxadiazol-3-yl)benzamide (250 mg, 0.6 mmol) in ethanol (7 mL), hydrazine hydrate (0.1 mL, 1.9 mmol) was added at 0-5° C. The reaction mixture was heated at 50° C. for 16 h. The reaction mixture was cooled to 25° C. and concentrated under reduced pressure. The crude product was purified by flash chromatography using 50% ethyl acetate in hexane to obtain N-methyl-N-(2-phenoxyethyl)-4-(5-(trifluoromethyl)-1h-1,2,4-triazol-3-yl)benzamide (106 mg, 0.3 mmol, 42.5% yield).

TABLE 3
The following compounds were prepared by the process analogous to that of the
compound 11

| Compound | | | |
|---|---|---|---|
| no. | Structure | Iupac name | Yield |
| 44 | 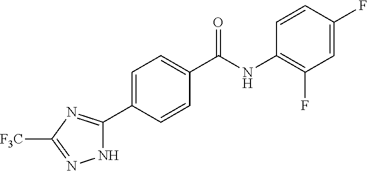 [SMILES: O=C(Nc1ccc(F)cc1F)c1ccc(-c2nc(C(F)(F)F)n[nH]2)cc1] | N-(2,4-difluorophenyl)-4-(3- (trifluoromethyl)-1h-1,2,4- triazol-5-yl)benzamide | 0.088 g, 25% |
| 45 | 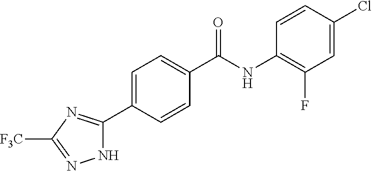 [SMILES: O=C(Nc1ccc(Cl)cc1F)c1ccc(-c2nc(C(F)(F)F)n[nH]2)cc1] | N-(4-chloro-2-fluorophenyl)-4- (3-(trifluoromethyl)-1h-1,2,4- triazol-5-yl)benzamide | 0.075 g, 22% yield |

### Example 4:—Preparation of N-methyl-N-(2-phenoxyethyl)-4-(5-(trifluoromethyl)-4,5-dihydro-1,2,4-oxadiazol-3-yl)benzamide (Compound 13)

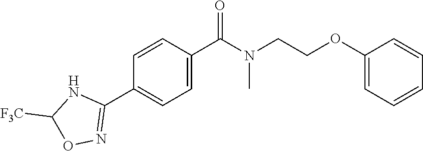 [SMILES: CN(CCOc1ccccc1)C(=O)c1ccc(C2=NOC(C(F)(F)F)N2)cc1]

To a stirred solution of N-methyl-N-(2-phenoxyethyl)-4-(5-(trifluoromethyl)-1,2,4-oxadiazol-3-yl)benzamide (400 mg, 1. mmol) in tetrahydrofuran (7 mL), borane-methyl sulfide complex (0.5 mL, 5.1 mmol) was added at −15° C. under nitrogen atmosphere. The reaction mixture was stirred at 0° C. for 30 minutes and at 25° C. for 30 min. The reaction was quenched by drop wise addition of methanol (5 ml) at 0° C. The reaction mixture was concentrated and the residue was purified by column chromatography on silica gel using 50% ethyl acetate in hexane as an eluent to obtain N-methyl-N-(2-phenoxyethyl)-4-(5-(trifluoromethyl)-4,5-dihydro-1,2,4-oxadiazol-3-yl)benzamide (262 mg, 0.6 mmol, 65.2% yield).

TABLE 4
The following compounds were prepared by the process analogous to that of the
compound 13.

| Compound | | | |
|---|---|---|---|
| no. | Structure | Iupac name | Yield |
| 28 | 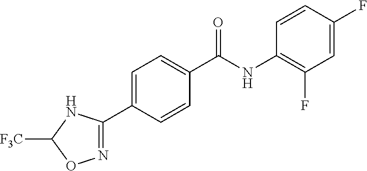 [SMILES: O=C(Nc1ccc(F)cc1F)c1ccc(C2=NOC(C(F)(F)F)N2)cc1] | N-(2,4-difluorophenyl)-4-(5- (trifluoromethyl)-4,5-dihydro- 1,2,4-oxadiazol-3-yl)benzamide | 0.1 g, 21.48% yield |
| 29 | 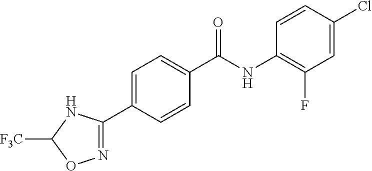 [SMILES: O=C(Nc1ccc(Cl)cc1F)c1ccc(C2=NOC(C(F)(F)F)N2)cc1] | N-(4-chloro-2-fluorophenyl)-4- (5-(trifluoromethyl)-4,5- dihydro-1,2,4-oxadiazol-3- yl)benzamide | 0.08 g, 23% yield |

### Example 5: Preparation of ((4-(5-(difluoromethyl)-1,2,4-oxadiazol-3-yl)phenyl)imino)(4-methoxyphenyl)(methyl)-λ⁶-sulfanone (Compound-15)

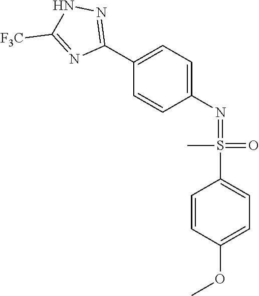 [SMILES: COc1ccc(S(C)(=O)=Nc2ccc(-c3n[nH]c(C(F)(F)F)n3)cc2)cc1]

### Step 1: 4-(((4-Methoxyphenyl)(methyl)(oxo)-λ⁶-sulfanylidene)amino)benzonitrile

To a stirred solution of p-iodobenzonitrile (1.5 g, 6.5 mmol) and toluene (20 mL), imino(4-methoxyphenyl)(methyl)-λ⁶-sulfanone (1.5 g, 7.8 mmol) and caesium carbonate (2.9 g, 9.1 mmol) were added under nitrogen atmosphere. The reaction mixture was degassed with nitrogen for 10 minutes and then 2,2′-bis(diphenylphosphino)-1,1′-binaphthyl (0.408 g, 0.655 mmol) was added followed by the addition of palladium(I)acetate (0.07 g, 0.3 mmol) and stirred at 105° C. for 18 h. After completion of the reaction, the reaction mixture was filtered through celite bed and diluted with ethyl acetate (30 mL). The ethyl acetate layer was washed three times with water (15 mL), separated the organic layer, dried over anhydrous sodium sulphate. The ethyl acetate layer was evaporated under reduced pressure. The crude residue was purified using column chromatography on silica gel using 50% ethyl acetate in hexane to obtain 4-(((4-methoxyphenyl)(methyl)(oxo)-λ⁶-sulfanylidene)amino)benzonitrile (1.8 g, 6.3 mmol, 96% yield).

### Step-2: N′-hydroxy-4-(((4-methoxyphenyl)(methyl)(oxo)-λ⁶-sulfanylidene)amino) benzimidamide

To a stirred solution of 4-(((4-methoxyphenyl)(methy)(oxo)-λ⁶-sulfanylidene)amino)benzonitrile (1.8 g, 6.3 mmol) and ethanol (20 mL), hydroxylamine hydrochloride (0.8 g, 11.3 mmol) and sodium bicarbonate (0.9 g, 11.3 mmol) were added at 25° C. and stirred at 70° C. for 16 h. The reaction mixture was filtered and concentrated to obtain N′-hydroxy-4-(((4-methoxyphenyl)(methyl)(oxo)-λ⁶-sulfanylidene)amino)benzimidamide (2 g, 6.26 mmol, 100% yield).

### Step-3: (4-Methoxyphenyl)(methyl)((4-(5-(trifluoromethyl)-1,2,4-oxadiazol-3-yl)phenyl)imino)-λ⁶-sulfanone

To a stirred solution of N′-hydroxy-4-(((4-methoxyphenyl)(methyl)(oxo)-λ⁶-sulfanylidene)amino)benzimidamide (1.5 g, 4.7 mmol) and tetrahydrofuran (15 mL), trifluoroacetic anhydride (0.9 mL, 7.1 mmol) was added at 0-5° C. under nitrogen atmosphere. The reaction mixture was then stirred at 25° C. for 18 h. The crude product was extracted into ethyl acetate (40 mL). The ethyl acetate layer was washed two times with saturated sodium bicarbonate solution (10 mL), dried over sodium sulphate and concentrated under reduced pressure. The crude product was purified by column chromatography on silica gel using hexane to 50% ethyl acetate in hexane to obtain (4-methoxyphenyl)(methyl)((4-(5-(trifluoromethyl)-1,2,4-oxadiazol-3-yl)phenyl)imino)-λ⁶-sulfanone (1.3 g, 3.3 mmol, 69.7% yield).

### Step 4: (4-Methoxyphenyl)(methyl)((4-(5-(trifluoromethyl)-1h-1,2,4-triazol-3-yl)phenyl)imino)-λ⁶-sulfanone

To a stirred solution of (4-methoxyphenyl)(methyl)((4-(5-(trifluoromethyl)-1,2,4-oxadiazol-3-yl)phenyl)imino)-λ⁶-sulfanone (0.3 g, 0.7 mmol) and ethanol (10 mL), hydrazine hydrate (0.2 g, 3.7 mmol) was added at 25° C. and stirred at 75° C. for 18 h. The reaction mixture was concentrated and residue was purified by column chromatography using 80% ethyl acetate in hexane to obtain (4-methoxyphenyl)(methyl)((4-(5-(trifluoromethyl)-1h-1,2,4-triazol-3-yl)phenyl)imino)-λ⁶-sulfanone (133 mg, 0.33 mmol, 44.4% yield).

TABLE 5
The following compounds were prepared by the process analogous to that of the
compound 15.

| Compound | | | |
|---|---|---|---|
| no. | Structure | Iupac name | Yield |
| 40 | 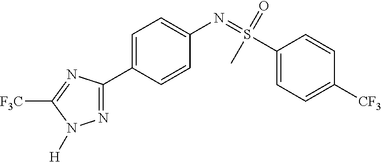 [SMILES: CS(=O)(=Nc1ccc(-c2n[nH]c(C(F)(F)F)n2)cc1)c1ccc(C(F)(F)F)cc1] | Methyl((4-(5-(trifluoromethyl)- 1h-1,2,4-triazol-3- yl)phenyl)imino)(4- (trifluoromethyl)phenyl)-λ⁶- sulfanone | 0.27 g, 54% yield |
| 41 | 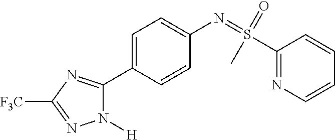 [SMILES: CS(=O)(=Nc1ccc(-c2nc(C(F)(F)F)n[nH]2)cc1)c1ccccn1] | Methyl(pyridin-2-yl)((4-(3- (trifluoromethyl)-1h-1,2,4- triazol-5-yl)phenyl)imino)-λ⁶- sulfanone | 0.18 g, 37% yield |

### Example 6:—Preparation of (4-methoxyphenyl)(methyl)((4-(5-(trifluoromethyl)-4,5-dihydro-1,2,4-oxadiazol-3-yl)phenyl)imino)-λ⁶-sulfanone (Compound 16)

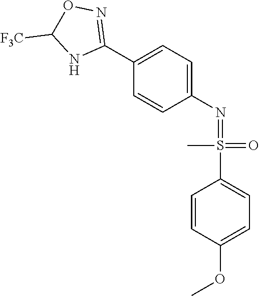 [SMILES: COc1ccc(S(C)(=O)=Nc2ccc(C3=NOC(C(F)(F)F)N3)cc2)cc1]

To a solution of (4-methoxyphenyl)(methyl)((4-(5-(trifluoromethyl)-1,2,4-oxadiazol-3-yl)phenyl)imino)-λ⁶-sulfanone (0.3 g, 0.7 mmol) and tetrahydrofuran (3 mL), borane-methyl sulfide complex (0.2 mL, 2.2 mmol) was added at −15° C., stirred at 0° C. for 30 minutes and then at 25° C. for 30 minutes. The reaction was quenched by drop wise addition of methanol (5 ml) at 0-5° C. The reaction mixture was concentrated and the residue Was purified by column chromatography on silica gel using 50% ethyl acetate in hexane to obtain the product (155 mg, yield 51%).

TABLE 6
The following compounds were prepared by the process analogous to that of the
compound 16.

| Compound | | | |
|---|---|---|---|
| no. | Structure | Iupac name | Yield |
| 24 | 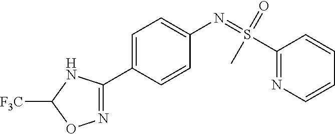 [SMILES: CS(=O)(=Nc1ccc(C2=NOC(C(F)(F)F)N2)cc1)c1ccccn1] | Methyl(pyridin-2-yl)((4-(5- (trifluoromethyl)-4,5-dihydro-1,2,4- oxadiazol-3-yl)phenyl)imino)-λ⁶- sulfanone | 0.17 g, 34% yield |
| 26 | 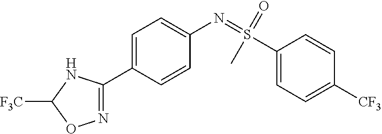 [SMILES: CS(=O)(=Nc1ccc(C2=NOC(C(F)(F)F)N2)cc1)c1ccc(C(F)(F)F)cc1] | Methyl((4-(5-(trifluoromethyl)-4,5- dihydro-1,2,4-oxadiazol-3- yl)phenyl)imino)(4- (trifluoromethyl)phenyl)-λ⁶- sulfanone | 0.25 g, 50% yield |

### Example 7:—Preparation of 3-(4-(phenylsulfonyl)phenyl)-5-(trifluoromethyl)-1h-1,2,4-triazole (Compound 9)

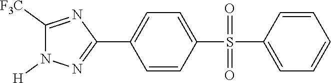 [SMILES: O=S(=O)(c1ccccc1)c1ccc(-c2n[nH]c(C(F)(F)F)n2)cc1]

### Step-1: 4-(phenylthio)benzonitrile (prb-cn205-08)

To a stirred solution of thiophenol (0.3 mL, 3.30 mmol) and N,N-dimethyl formamide (10 mL), potassium tert-butoxide (0.5 g, 4.1 mmol) was added at 0-5° C. and stirred at 25° C. for 15 minutes. The reaction mixture was cooled to 0-5° C. followed by addition of 4-bromobenzonitrile (0.5 g, 2.7 mmol). The reaction mixture was stirred at 80° C. for 2 h. The reaction mixture was cooled to 25° C., extracted with ethyl acetate (30 mL). The ethyl acetate layer was washed with water (10 mL), dried over sodium sulphate and concentrated to obtain 4-(phenylthio)benzonitrile (0.48 g).

### Step-2: N′-hydroxy-4-(phenylthio)benzimidamide

To a solution of 4-(phenylthio)benzonitrile (5.8 g, 27.5 mmol) in ethanol (30 mL) and water (90 mL), hydroxylamine hydrochloride (4.8 g, 68.6 mmol) and triethyl amine (9.5 mL, 68.6 mmol) were added at 25° C. The reaction mixture was stirred at 65° C. for 6 h. The reaction mixture was concentrated under reduced pressure to obtain N′-hydroxy-4-(phenylthio)benzimidamide.

### Step-3: 3-(4-(Phenylthio)phenyl)-5-(trifluoromethyl)-1,2,4-oxadiazole

To a solution of N′-hydroxy-4-(phenylthio)benzimidamide (6.5 g, 26.6 mmol) in tetrahydrofuran (53.2 mL), trifluoroacetic acid (5.6 mL, 39.9 mmol) was added at 0-5° C. and stirred at 25° C. for 18 hs. The reaction mixture was concentrated and purified by column chromatography on silica gel using 40% ethyl acetate in hexane to obtain 3-(4-(phenylthio)phenyl)-5-(trifluoromethyl)-1,2,4-oxadiazole. (Yield—6 g, 70% yield).

### Step-4: 3-(4-(Phenylsulfonyl)phenyl)-5-(trifluoromethyl)-1,2,4-oxadiazole

To a stirred solution of 3-(4-(phenylthio)phenyl)-5-trifluoromethyl)-1,2,4-oxadiazole (2.2 g, 6.8 mmol) in dichloro methane (25 mL), m-chloroperbenzoic acid (3.1 g, 10.9 mmol) was added at 0-5° C. under nitrogen atmosphere. The white suspension was stirred at 25° C. for 16 h. After completion of the reaction, the crude product was extracted into dichloro methane (50 mL). The dichloro methane layer was washed twice with saturated sodium bicarbonate solution (20 mL), dried over anhydrous sodium sulphate and concentrated under reduced pressure. The crude product was purified by column chromatography on silica gel using 40% ethyl acetate in hexane. The pure fractions were combined and concentrated to obtain 3-(4-(phenylsulfonyl)phenyl)-5-(trifluoromethyl)-1,2,4-oxadiazole (1.4 g, 4.1 mmol, 60.9% yield).

### Step-5: 3-(4-(Phenylsulfonyl)phenyl)-5-(trifluoromethyl)-1h-1,2,4-triazole

To a stirred solution of 3-(4-(phenylsulfonyl)phenyl)-5-(trifluoromethyl)-1,2,4-oxadiazole (0.7 g, 1.9 mmol) in ethanol (10 mL), hydrazine hydrate (0.5 mL, 9.8 mmol) was added at 0-5° C. The reaction mixture was heated at 65° C. for 16 h. The reaction mixture was concentrated under reduced pressure and the residue was purified by column chromatography on silica gel to obtain 3-(4-(phenylsulfonyl)phenyl)-5-(trifluoromethyl)-1h-1,2,4-triazole (310 mg, 0.8 mmol, 44.4% yield).

### Example 8:—Preparation of 3-(4-(phenylsulfonyl)phenyl)-5-(trifluoromethyl)-4,5-dihydro-1,2,4-oxadiazole (Compound 12)

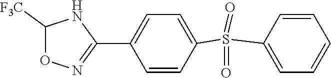 [SMILES: O=S(=O)(c1ccccc1)c1ccc(C2=NOC(C(F)(F)F)N2)cc1]

To a stirred solution of 3-(4-(phenylsulfonyl)phenyl)-5-(trifluoromethyl)-1,2,4-oxadiazole (450 mg, 1.3 mmol) in tetrahydrofuran (6 mL), borane dimethyl sulfide complex (603 μl, 6.3 mmol) was added at 0° C. under nitrogen atmosphere. The reaction mixture was stirred at 0° C. for 30 minutes. The reaction mixture was quenched by drop wise addition of methanol (5 mL). The reaction mixture was concentrated and the residue was purified by column chromatography to obtain 3-(4-(phenylsulfonyl)phenyl)-5-(trifluoromethyl)-4,5-dihydro-1,2,4-oxadiazole (172 mg, 0.5 mmol, 38.0% yield).

### Example 9: Preparation of 1-methyl-5-(4-(phenylsulfonyl)phenyl)-3-(trifluoromethyl)-1h-1,2,4-triazole (Compound 34)

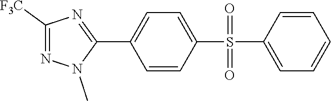 [SMILES: Cn1nc(C(F)(F)F)nc1-c1ccc(S(=O)(=O)c2ccccc2)cc1]

To a stirred solution of 3-(4-(phenylsulfonyl)phenyl)-5-(trifluoromethyl)-1,2,4-oxadiazole (300 mg, 0.8 mmol) in ethanol (8 mL) was added methyl hydrazine (0.4 mL, 6.7 mmol) at 0-5° C. The reaction mixture was heated at 70° C. for 16 h. After completion of the reaction, the volatiles were evaporated and the residue was purified by flash column chromatography to get 1-methyl-5-(4-(phenylsulfonyl)phenyl)-3-(trifluoromethyl)-1h-1,2,4-triazole (152 mg, 0.4 mmol, 48.9% yield)

### Example 10:—Preparation of 5-(difluoromethyl)-3-(4-(phenylsulfonyl)phenyl)-1,2,4-oxadiazole (Compound 4)

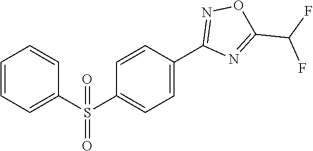 [SMILES: O=S(=O)(c1ccccc1)c1ccc(-c2noc(C(F)F)n2)cc1]

### Step 1: 5-(Difluoromethyl)-3-(4-(phenylthio)phenyl)-1,2,4-oxadiazole

To a solution of (z)-N′-hydroxy-4-(phenylthio)benzimidamide (330 mg, 1.3 mmol) in tetrahydrofuran (4 mL), trifluoroacetic anhydride (0.3 mL, 2.1 mmol) was added at 0-5° C. then stirred at 25° C. for 16 h. The reaction mixture was poured onto saturated solution of sodium carbonate (4 mL) at 0-5° C. The aqueous layer was extracted three times with dichloromethane (25 mL). The combined dichloromethane layers were washed with water (25 mL), brine solution (25 mL), dried over anhydrous sodium sulphate and evaporated under reduced pressure to obtain the residue. The residue was purified by flash chromatography using 40% ethyl acetate in hexane to give the title compound (0.4 g, 1.6 mmol, 92% yield).

### Step 2: 5-(Difluoromethyl)-3-(4-(phenylsulfonyl)phenyl)-1,2,4-oxadiazole

To a stirred solution of 5-(difluoromethyl)-3-(4-(phenylthio)phenyl)-1,2,4-oxadiazole (460 mg, 1.5 mmol) in dichloromethane (4.6 mL), meta-chloro perbenzoic acid (745 mg, 3.1 mmol) was added at 0-5° C. and stirred for 24 h at 25° C. The reaction mixture was poured onto saturated potassium carbonate solution (4 mL) at 0-5° C. The aqueous layer was extracted three times with dichloromethane (15 mL). The combined dichloromethane layers were washed with water (25 mL), brine solution (25 mL), dried over anhydrous sodium sulphate and evaporated under reduced pressure to obtain the residue. The residue was purified by flash chromatography using 40% ethyl acetate in hexane as an eluent to obtain 5-(difluoromethyl)-3-(4-(phenylsulfonyl)phenyl)-1,2,4-oxadiazole (0.368 mg, 1.5 mmol, 72% yield).

### Example 11: Preparation of N-(4-(5-(difluoromethyl)-1,2,4-oxadiazol-3-yl)benzyl)-N-methoxybenzamide (Compound 2)

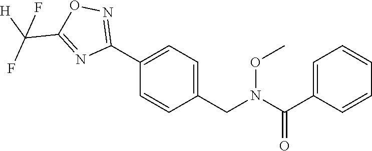 [SMILES: CON(Cc1ccc(-c2noc(C(F)F)n2)cc1)C(=O)c1ccccc1]

### Step 1: N′-hydroxy-4-methylbenzimidamide

To a stirred solution of 4-methylbenzonitrile (1 g, 8.5 mmol) in ethanol (10 mL), 50% aqueous hydroxylamine (1.1 mL, 17.1 mmol) was added at 25° C. and stirred at 78° C. for 18 h. The reaction mixture was concentrated under reduced pressure to get N′-hydroxy-4-methylbenzimidamide (1.3 g, 8.5 mmol, 100% yield).

### Step 2: 5-(Difluoromethyl)-3-(p-tolyl)-1,2,4-oxadiazole

To a stirred suspension of N′-hydroxy-4-methylbenzimidamide (1.3 g, 8.6 mmol) in tetrahydrofuran (15 mL), 2,2-difluoroacetic anhydride (1.2 mL, 11.2 mmol) was added at 0-5° C. The reaction mixture was stirred at 25° C. for 16 h. After completion of the reaction, the crude product was extracted into dichloromethane (30 mL). The dichloromethane layer was washed twice with saturated sodium bicarbonate solution (10 mL), dried over anhydrous sodium sulphate and concentrated under reduced pressure. The crude product was purified by column chromatography on silica gel using 50% ethyl acetate in hexane to obtain 5-(difluoromethyl)-3-(p-tolyl)-1,2,4-oxadiazole (1.4 g, 74% yield).

### Step 3: 3-(4-(Bromomethyl)phenyl)-5-(difluoromethyl)-1,2,4-oxadiazole

To a stirred solution of 5-(difluoromethyl)-3-(p-tolyl)-1,2,4-oxadiazole (1.4 g, 6.4 mmol) and chloroform (30 mL), N-bromosuccinimide (1.4 g, 7.7 mmol) was added and stirred at 65° C. for 15 minutes. Azobisisobutyronitrile (0.2 g, 0.9 mmol) was added to the reaction mixture and stirred at 65° C. for 3 h. After completion of the reaction, the crude product was extracted with dichloromethane (30 mL), dichloromethane layer was washed two time with saturated solution of sodium bicarbonate (10 mL), dried over sodium sulphate and concentrated under reduced pressure to obtain 3-(-(4-(bromomethyl)phenyl)-5-(difluoromethyl)-1,2,4-oxadiazole (1.5 g, 81%).

### Step 4: N-(4-(5-(difluoromethyl)-1,2,4-oxadiazol-3-yl)benzyl)-o-methylhydroxylamine

To a stirred solution of 3-(4-(bromomethyl)phenyl)-5-(difluoromethyl)-1,2,4-oxadiazole (1.0 g, 3.4 mmol) and dimethylformamide (10 mL), di-isopropyl ethyl amine (2.4 mL, 13.8 mmol) and o-methoxyamine hydrochloride (0.7 g, 8.6 mmol) were added at 25° C. The reaction mixture was stirred at 25° C. for 16 h. After completion of the reaction, the crude product was extracted with ethyl acetate (30 mL). The ethyl acetate layer was washed with water (10 ML), dried over anhydrous sodium sulphate and concentrated under reduced pressure. The product was purified by column chromatography on silica gel using eluent 50% ethyl acetate in hexane to obtain N-(4-(5-(difluoromethyl)-1,2,4-oxadiazol-3-yl)benzyl)-o-methylhydroxylamine (0.8 g, 63.4% yield).

### Step 5: N-(4-(5-(difluoromethyl)-1,2,4-oxadiazol-3-yl)benzyl)-N-methoxybenzamide

To a stirred solution of N-(4-(5-(difluoromethyl)-1,2,4-oxadiazol-3-yl)benzyl)-o-methylhydroxylamine (0.2 g, 0.7 mmol) in dichloromethane (5 mL), di-isopropylethyl amine (0.3 ml, 1.5 mmol) and benzoyl chloride (0.1 mL, 1.1 mmol) were added at 0-5° C. under nitrogen atmosphere. The reaction mixture was stirred at 25° C. for 16 h. After completion of the reaction, the crude product was extracted with ethyl acetate (30 mL). The ethyl acetate layer was washed with saturated solution of sodium bicarbonate (10 mL), dried over anhydrous sodium sulphate and concentrated under reduced pressure. The crude product was purified by column chromatography on silica gel using 50% ethyl acetate in hexane to obtain N-(4-(5-(difluoromethyl)-1,2,4-oxadiazol-3-yl)benzyl)-N-methoxybenzamide (0.2 gm, 88% yield).

### Example 12:—Preparation of N-(4-(5-(trifluoromethyl)-1h-1,2,4-triazol-3-yl)benzyl)pivalamide (Compound 3)

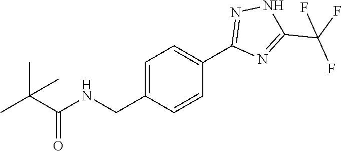 [SMILES: CC(C)(C)C(=O)NCc1ccc(-c2n[nH]c(C(F)(F)F)n2)cc1]

### Step 1: N′-hydroxy-4-methylbenzimidamide

To a stirred suspension of 4-methylbenzonitrile (1 g, 8.5 mmol) and ethanol (6.2 mL), hydroxylamine hydrochloride (1.2 g, 17.1 mmol), potassium carbonate (1.9 g, 13.8 mmol) and 8-hydroxyquinoline (0.02 g, 0.13 mmol) were added at 25° C. The resulting suspension was stirred at 80° C. for 4 h. Ethanol was removed under reduced pressure. The pH of the reaction mixture was adjusted to 8 by 10% aqueous solution of hydrochloric acid at 5-8° C. The precipitate was filtered and washed with ice cold water (5 mL), dried under reduced pressure to obtain N′-hydroxy-4-methylbenzimidamide (0.9 g, 5.9 mmol, 70.2% yield).

### Step 2: 3-(p-Tolyl)-5-(trifluoromethyl)-1,2,4-oxadiazole

To a stirred solution of N′-hydroxy-4-methylbenzimidamide (0.9 g, 5.9 mmol) in tetrahydrofuran (15 mL), trifluoroacetic acid (1.3 mL, 8.9 mmol) was added at 0-5° C. under nitrogen atmosphere. The reaction mixture was then stirred at 25° C. for 6 h. Ethyl acetate (50 mL) was added to the reaction mixture followed by the cautious addition of saturated solution of sodium bicarbonate. Ethyl acetate layer was separated washed with water (10 mL), dried over anhydrous sodium sulphate and concentrated under reduced pressure. The crude product was purified by column chromatography on silica gel using 10% ethyl acetate in hexane to obtain 3-(p-tolyl)-5-(trifluoromethyl)-1,2,4-oxadiazole (1.2 g, yield 88%).

### Step 3: 3-(4-(Bromomethyl)phenyl)-5-(trifluoromethyl)-1,2,4-oxadiazole

To a stirred solution of 3-(p-tolyl)-5-(trifluoromethyl)-1,2,4-oxadiazole (13.2 g, 57.9 mmol) and carbon tetrachloride (100 mL), N-bromosuccinimide (11.33 g, 63.6 mmol) was added and stirred at 70° C. for 10 minutes after which a brown solution was obtained. Benzoyl peroxide (2.0 g, 5.7 mmol) was added and the reaction mixture was stirred at 65° C. for 15 h. The crude product was extracted with dichloromethane (20 mL). The dichloromethane layer was washed with saturated solution of sodium bicarbonate (10 mL), dried over anhydrous sodium sulphate and concentrated under reduced pressure. The crude product was purified by column chromatography on silica gel using 10% ethyl acetate in hexane to obtain 3-(4-(bromomethyl)phenyl)-5-(trifluoromethyl)-1,2,4-oxadiazole (13.8 g, 78% yield).

### Step 4: 2-(4-(5-(Trifluoromethyl)-1,2,4-oxadiazol-3-yl)benzyl)isoindoline-1,3-dione

To a stirred solution of 3-(4-(bromomethyl)phenyl)-5-(trifluoromethyl)-1,2,4-oxadiazole (0.2 g, 0.6 mmol) and N,N-dimethylformamide (2 mL), potassium phthalimide (0.2 g, 0.9 mmol) was added at 0-5° C. under nitrogen. The reaction mixture was stirred at 65° C. for 4 h. The reaction mixture was cooled to 25° C. and poured over crushed ice with stirring. The white precipitate obtained was filtered, washed with water (10 mL) and dried under reduced pressure to obtain 2-(4-(5-(trifluoromethyl)-1,2,4-oxadiazol-3-yl)benzyl)isoindoline-1,3-dione (0.2 g, 0.56 mmol, 86% yield).

### Step 5: (4-(5-(Trifluoromethyl)-1h-1,2,4-triazol-3-yl)phenyl)methanamine

To a stirred suspension of 2-(4-(5-(trifluoromethyl)-1,2,4-oxadiazol-3-yl)benzyl)isoindoline-1,3-dione (2.1 g, 5.6 mmol) and ethanol (20 mL), hydrazine monohydrate (2.8 g, 56.3 mmol) was added and the reaction mixture was stirred at 75° C. for 3 h. The reaction mixture was cooled to 25° C. The white precipitate was filtered, washed with ethanol (5 mL). The combined filtrates were concentrated to obtain (4-(5-(trifluoromethyl)-1h-1,2,4-triazol-3-yl)phenyl)methanamine (1.2 g, 4.9 mmol, 88% yield).

### Step 6: N-(4-(5-(trifluoromethyl)-1h-1,2,4-triazol-3-yl)benzyl)pivalamide

To a stirred solution of (4-(5-(trifluoromethyl)-1h-1,2,4-triazol-3-yl)phenyl)methanamine (1 g, 4.1 mmol) and dichloromethane (20 mL), pyridine (1.6 mL, 20.6 mmol) and triethyl amine (1.7 mL, 12.3 mmol) were added at 25° C. The reaction mixture was cooled to 0-5° C. and pivaloyl chloride (0.7 mL, 6.1 mmol) was added to it. The reaction mixture was stirred at 25° C. for 2 h. After completion of reaction, saturated solution of sodium bicarbonate (10 mL) was added into the reaction mixture. The crude product was extracted two times with dichloromethane (20 mL). The combined dichloromethane layers were washed with water (20 mL), dried over anhydrous sodium sulphate and concentrated under reduced pressure. The crude product was purified by column chromatography on silica using 40% ethyl acetate in hexane to obtain N-(4-(5-(trifluoromethyl)-1h-1,2,4-triazol-3-yl)benzyl)pivalamide (0.3 g, 22% yield).

### Step 7: Purification of N-(4-(5-(trifluoromethyl)-1h-1,2,4-triazol-3-yl)benzyl)pivalamide

A mixture of N-(4-(5-(trifluoromethyl)-1h-1,2,4-triazol-3-yl)benzyl)pivalamide (0.3 g, 1.1 mmol) and diethyl ether (10 mL) was stirred at 25° C. for 2 h and filtered. Obtained powder was dried under reduced pressure to get N-(4-(5-(trifluoromethyl)-1h-1,2,4-triazol-3-yl)benzyl)pivalamide (0.2 g, 0.7 mmol, 70% yield).

### Example 13:—Preparation of 4-(5-(difluoromethyl)-1,2,4-oxadiazol-3-yl)-N-methyl-N-(2-phenoxyethyl)benzamide (Compound 17)

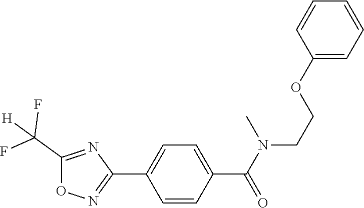 [SMILES: CN(CCOc1ccccc1)C(=O)c1ccc(-c2noc(C(F)F)n2)cc1]

### Step 1: 4-Cyano-N-methyl-N-(2-phenoxyethyl)benzamide

To a stirred solution of 4-cyanobenzoic acid (720 mg, 4.8 mmol) and dichloromethane (2 mL), 1-[bis(dimethylamino)methylene]-1h-1,2,3-triazolo[4,5-b]pyridinium 3-oxide hexafluorophosphate (1861 mg, 4.8 mmol) and di-isopropylethylamine (1.4 mL, 8.1 mmol) were added and stirred for 20 min followed by addition of N-methyl-2-phenoxyethan-1-amine (700 mg, 4.6 mmol). The stirring was continued for 16 h at 25° C. After completion of the reaction, the reaction mixture was diluted with ethyl acetate (20 mL). The ethyl acetate layer was separated, washed with saturated solution of sodium bicarbonate (30 mL), 10% dilute hydrochloric acid (30 mL), water (20 mL) and brine solution (20 mL). The ethyl acetate layer was dried over anhydrous sodium sulphate and concentrated under reduced pressure. The crude product was purified with column chromatography on silica gel using 40% ethyl acetate in hexane to obtain methyl 4-(5-(difluoromethyl)-1,2,4-oxadiazol-3-yl)benzoate (0.6 g, 2.1 mmol, 52.5% yield).

### Step 2: 4-(N′-hydroxycarbamimidoyl)-N-methyl-N-(2-phenoxyethyl)benzamide

To a stirred solution of 4-cyano-N-methyl-N-(2-phenoxyethyl)benzamide (1.1 g, 3.9 mmol) in methanol (11 mL), sodium carbonate (0.7 g, 7.1 mmol), hydroxylamine hydrochloride (0.5 g, 7.1 mmol) were added and stirred at 25° C. for 16 h. After completion of the reaction, water (100 mL) was added and the reaction mixture was extracted twice with ethyl acetate (30 mL). The combined ethyl acetate layers were washed with water (10 mL), brine solution (10 mL), dried over anhydrous sodium sulphate, and concentrated under reduced pressure to obtain 4-(N′-hydroxycarbamimidoyl)-N-methyl-N-(2-phenoxyethyl)benzamide (0.8 g, 2.5 mmol, 65%).

### Step 3: 4-(5-(Difluoromethyl)-1,2,4-oxadiazol-3-yl)-N-methyl-N-(2-phenoxyethyl)benzamide

To a stirred solution of 4-(N′-hydroxycarbamimidoyl)-N-methyl-N-(2-phenoxyethyl)benzamide (400 mg, 1.2 mmol) in tetrahydrofuran (8.5 mL), difluoroacetic anhydride (0.3 mL, 2.3 mmol) was added at 0-5° C. and the reaction mixture was stirred for 16 h at 25° C. After completion of the reaction, the reaction mixture was poured onto saturated solution of sodium carbonate (4 mL) at 0-5° C. The aqueous layer was extracted twice with dichloromethane (10 mL). The combined dichloromethane layers were washed with water (10 mL), brine solution (10 mL), dried over anhydrous sodium sulphate and evaporated under reduced pressure to obtain the residue, which was purified by flash chromatography using 40% ethyl acetate in hexane as eluent to obtain the title compound (0.3 g, 1.2 mmol, 72.4% yield).

### Example 14:—Preparation of ((4-(5-(difluoromethyl)-4,5-dihydro-1,2,4-oxadiazol-3-yl)benzyl)imino)(methyl)(phenyl)-λ⁶-sulfanone (Compound 27)

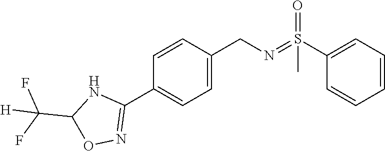 [SMILES: CS(=O)(=NCc1ccc(C2=NOC(C(F)F)N2)cc1)c1ccccc1]

### Step 1:—4-(((methyl(oxo)(phenyl)-ko-sulfaneylidene)amino)methyl)benzonitrile

To a stirred solution of imino(methyl)(phenyl)-λ⁶-sulfanone (1.2 g, 7.7 mmol), 4-(bromomethyl)benzonitrile (1.6 g, 8.5 mmol) in N,N-dimethylformamide (8 mL) was added potassium tert-butoxide (1.7 g, 15.4 mmol) at 0-5° C. under nitrogen atmosphere. The reaction mixture was stirred at 0-5° C. for 15 minutes. Saturated aqueous ammonium chloride solution (10 mL) and ethyl acetate (40 mL) were added to the reaction mixture and stirred at 0-5° C. for 10 minutes. The ethyl acetate layer was isolated, washed with water (20 mL), dried over anhydrous sodium sulphate and concentrated under reduced pressure to obtain 4-(((methyl(oxo)(phenyl)-λ⁶-sulfanylidene)amino)methyl)benzonitrile (1.9 g, 7. mmol, 91% yield).

### Step 2: N′-hydroxy-4-(((methyl(oxo)(phenyl)-ka-sulfaneylidene)amino)methyl)benzimidamide

To a stirred solution of 4-(((methyl(oxo)(phenyl)-λ⁶-sulfanylidene)amino)methyl)benzonitrile (1.9 g, 7 mmol) in ethanol (10 mL), hydroxylamine hydrochloride (0.9 g, 14 mmol) and sodium bicarbonate (1.1 g, 14.1 mmol) were added at room temperature. The reaction mixture was heated to 65° C. for 12 h. After completion of the reaction, the reaction mixture was filtered through sintered funnel. The filtrate was evaporated under reduced pressure to obtain N′-hydroxy-4-(((methyl(oxo)(phenyl)-λ⁶-sulfanylidene)amino)methyl)benzimidamide (2.0 g, 6.5 mmol, 94% yield).

### Step 3: ((4-(5-(Difluoromethyl)-1,2,4-oxadiazol-3-yl)benzyl)imino)(methyl)(phenyl)-λ⁶-sulfanone

To a stirred solution of N′-hydroxy-4-(((methyl(oxo)(phenyl)-λ⁶-sulfanylidene)amino)methyl)benzimidamide (2.0 g, 6.5 mmol) in tetrahydrofuran (30 mL), difluoroacetic anhydride (0.7 mL, 6.5 mmol) was added at 0-5° C. under nitrogen atmosphere. The reaction mixture was stirred at 25° C. for 16 h. After completion of the reaction, the reaction mixture was diluted with ethyl acetate (30 mL) and washed with saturated sodium bicarbonate (30 ml) solution, dried over anhydrous sodium sulphate and concentrated under reduced pressure. The crude product was purified by column chromatography using 25% ethyl acetate in hexane on silica gel to obtain ((4-(5-(difluoromethyl)-1,2,4-oxadiazol-3-yl)benzyl)imino)(methyl)(phenyl)-λ⁶-sulfanone (0.5 g, 1.4 mmol, 20.8% yield).

### Step 4:—((4-(5-(Difluoromethyl)-4,5-dihydro-1,2,4-oxadiazol-3-yl)benzyl)imino)(methyl)(phenyl)-λ⁶-sulfanone (Compound 27)

To a stirred solution of ((4-(5-(difluoromethyl)-1,2,4-oxadiazol-3-yl)benzyl)imino)(methyl)(phenyl)-λ⁶-sulfanone (0.35 g, 0.9 mmol) in tetrahydrofuran (7 mL) was added borane dimethyl sulfide complex (0.3 mL, 2.8 mmol) drop wise at 0° C. The resulting reaction mixture was stirred for 2 h at 0° C. The reaction was allowed to stir for 2 h at 0° C. After completion of the reaction, reaction mixture was quenched with methanol, the reaction mixture was evaporated under reduced pressure, the crude product was purified using column chromatography using 50% ethyl acetate in hexane on silica gel to obtain ((4-(5-(difluoromethyl)-4,5-dihydro-1,2,4-oxadiazol-3-yl)benzyl)imino)(methyl)(phenyl)-λ⁶-sulfanone (100 mg, 0.3 mmol, 28.4% yield).

### Example 15:—Preparation of ((4-(3-(difluoromethyl)-1h-1,2,4-triazol-5-yl)benzyl)imino)(methyl)(phenyl)-λ⁶-sulfanone (Compound 42)

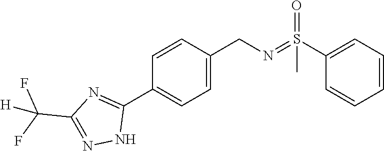 [SMILES: CS(=O)(=NCc1ccc(-c2nc(C(F)F)n[nH]2)cc1)c1ccccc1]

To a stirred solution of ((4-(5-(difluoromethyl)-1,2,4-oxadiazol-3-yl)benzyl)imino)(methyl)(phenyl)-λ⁶-sulfanone (0.2 g, 0.4 mmol) in ethanol (12 mL) was added hydrazine hydrate (0.034 mL, 0.7 mmol) at 25° C. The reaction mixture was heated at 40°. The reaction was allowed to stir for 12 h at 40° C. After completion of the reaction, reaction mixture was quenched with methanol, the reaction mixture was evaporated under reduced pressure, the crude product was purified using column chromatography using 50% ethyl acetate in hexane) on silica gel to obtain ((4-(3-(difluoromethyl)-1h-1,2,4-triazol-5-yl)benzyl)imino)(methyl)(phenyl)-λ⁶-sulfanone (96 mg, 0.2 mmol, 56.6% yield).

TABLE 7
The following compound was prepared from intermediate methyl(pyridin-3-yl)((4-(5-
(trifluoromethyl)-1,2,4-oxadiazol-3-yl)benzyl)imino)-λ⁶-sulfanone by procedure analogous to
that of the compound 42.

| Compound | | | |
|---|---|---|---|
| no. | Structure | Iupac name | Yield |
| 47 | 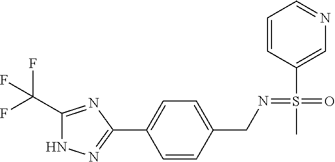 [SMILES: CS(=O)(=NCc1ccc(-c2n[nH]c(C(F)(F)F)n2)cc1)c1cccnc1] | Methyl(pyridin-3-yl)((4-(5- (trifluoromethyl)-1h-1,2,4- triazol-3-yl)benzyl)imino)- λ⁶-sulfanone | 0.07 g, 23% yield |

### Example 16:—Preparation of methyl(p-tolyl)((4-(5-(trifluoromethyl)-4,5-dihydro-1,2,4-oxadiazol-3-yl)benzyl)imino)-λ⁶-sulfanone (Compound 30)

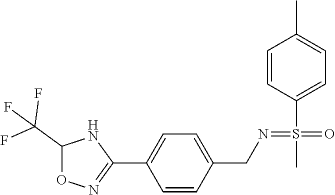 [SMILES: Cc1ccc(S(C)(=O)=NCc2ccc(C3=NOC(C(F)(F)F)N3)cc2)cc1]

### Step-1:—Methyl(p-tolyl)((4-(5-(trifluoromethyl)-1,2,4-oxadiazol-3-yl)benzyl)imino)-λ⁶-sulfanone

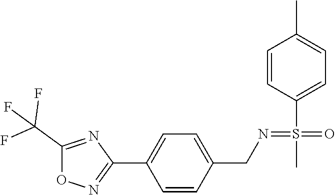 [SMILES: Cc1ccc(S(C)(=O)=NCc2ccc(-c3noc(C(F)(F)F)n3)cc2)cc1]

To a stirred solution of N′-hydroxy-4-(((methyl(oxo)(p-tolyl)-λ⁶-sulfanylidene)amino)methyl)benzimidamide (0.9 g, 2.8 mmol) in tetrahydrofuran (30 mL) was added trifluoroacetic anhydride (0.4 mL, 2.8 mmol) at 0° C. under nitrogen atmosphere. The reaction mixture was allowed to stir at 25° C. for 16 h. After completion of the reaction, reaction mixture was diluted with ethyl acetate (40 mL) and washed with ice cold sodium bicarbonate solution (40 mL). The organic layer was dried over anhydrous sodium sulphate, evaporated under reduced pressure to get crude compound. The crude product was purified chromatography to obtain methyl(p-tolyl)((4-(5-(trifluoromethyl)-1,2,4-oxadiazol-3-yl)benzyl)imino)-λ⁶-sulfanone (100 mg, 0.2 mmol, 9.00% yield).

### Step 2:—Methyl(p-tolyl)((4-(5-(trifluoromethyl)-4,5-dihydro-1,2,4-oxadiazol-3-yl)benzyl)imino)-λ⁶-sulfanone

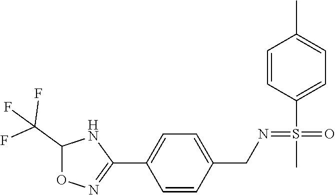 [SMILES: Cc1ccc(S(C)(=O)=NCc2ccc(C3=NOC(C(F)(F)F)N3)cc2)cc1]

To a stirred solution of methyl(p-tolyl)((4-(5-(trifluoromethyl)-1,2,4-oxadiazol-3-yl)benzyl)imino)-λ⁶-sulfanone (0.2 g, 0.5 mmol) in tetrahydrofuran (30 mL) was added borane-methyl sulfide complex (0.1 mL, 1.2 mmol) at 0° C. under nitrogen atmosphere. The reaction mixture was allowed to stir at 25° C. for 16 h. After completion of the reaction, the reaction mixture was diluted with ethyl acetate (40 mL) and washed with ice cold sodium bicarbonate solution (40 mL). The organic layer was dried over anhydrous sodium sulphate, evaporated under reduced pressure to get crude compound. The crude product was purified by chromatography to obtain methyl(p-tolyl)((4-(5-(trifluoromethyl)-4,5-dihydro-1,2,4-oxadiazol-3-yl)benzyl)imino)-λ⁶-sulfanone (66.7 mg, 0.2 mmol, 33% yield).

### Example 17:—Preparation of 4-(5-(difluoromethyl)-4,5-dihydro-1,2,4-oxadiazol-3-yl)-N-((4-fluorophenyl)(methyl)(oxo)-λ⁶-sulfaneylidene)benzamide (Compound 19)

### Step 1:—4-Cyano-N-((4-fluorophenyl)(methyl)(oxo)-λ⁶-sulfaneylidene)benzamide

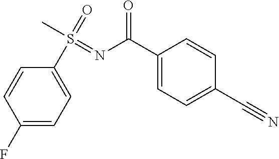 [SMILES: CS(=O)(=NC(=O)c1ccc(C#N)cc1)c1ccc(F)cc1]

To a stirred solution of 4-cyanobenzoic acid (347 mg, 2.4 mmol) in dichloromethane (3.4 mL) was added 1-(3-dimethylaminopropyl)-3-ethylcarbodiimide hydrochloride (753 mg, 3.9 mmol), 4-dimethylaminopyridine (719 mg, 5.9 mmol) and stirred for 20 min followed by the addition of (4-fluorophenyl)(imino)(methyl)-λ⁶-sulfanone (340 mg, 1.9 mmol). The reaction mixture was stirred for 24 h at 25° C. After completion of the reaction, the reaction mixture was poured onto aqueous saturated potassium carbonate solution at 0-5° C. The aqueous layer was extracted thrice with dichloromethane (100 mL). The combined dichloromethane layers were washed with water (25 mL), saturated brine solution (25 mL), dried over anhydrous sodium sulphate and evaporated under reduced pressure to get the residue, which was then purified by column chromatography using 20% ethyl acetate in hexane as an eluent. (0.35 g, 58%)

### Step-2:—N-((4-fluorophenyl)(methyl)(oxo)-λ⁶-sulfaneylidene)-4-(N′-hydroxycarbamimidoyl)benzamide

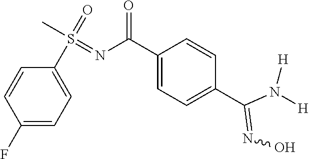 [SMILES: CS(=O)(=NC(=O)c1ccc(C(N)=NO)cc1)c1ccc(F)cc1]

To a solution of 4-cyano-N-((4-fluorophenyl)(methyl)(oxo)-λ⁶-sulfanylidene)benzamide (345 mg, 1.1 mmol) in a mixture of methanol (8 mL) and water (2 mL) was added sodium carbonate (218 mg, 2.0 mmol), hydroxylamine hydrochloride (143 mg, 2.0 mmol) then stirred at 25° C. for 16 h. After completion of the reaction, water (100 mL) was added, extracted twice with ethyl acetate (100 mL). Combined organic layers were washed with water (25 mL), saturated brine solution (25 mL), dried over anhydrous sodium sulphate and concentrated under reduced pressure to obtain N-((4-fluorophenyl)(methyl)(oxo)-λ⁶-sulfaneylidene)-4-(N′-hydroxycarbamimidoyl)benzamide (0.3 g, 1.1 mmol, 81% yield).

### Step 3:—4-(5-(Difluoromethyl)-1,2,4-oxadiazol-3-yl)-N-((4-fluorophenyl)(methyl)(oxo)-λ⁶-sulfaneylidene)benzamide

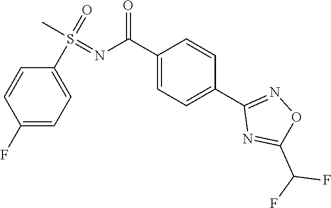 [SMILES: CS(=O)(=NC(=O)c1ccc(-c2noc(C(F)F)n2)cc1)c1ccc(F)cc1]

To a stirred solution of N-((4-fluorophenyl)(methyl)(oxo)-λ⁶-sulfanylidene)-4-(N′-hydroxycarbamimidoyl)benzamide (430 mg, 1.3 mmol) in tetrahydrofuran (4.3 mL) was added difluoroacetic anhydride (0.3 mL, 1.9 mmol) at 0-5° C. then stirred for 24 h at 25° C. After completion of the reaction, the reaction mixture was poured onto aqueous saturated sodium bicarbonate solution (10 ml) at 0-5° C. The aqueous layer was extracted thrice with dichloromethane (25 mL). The combined dichloromethane layers were washed with water (25 mL), brine solution (25 mL), dried over anhydrous sodium sulphate and evaporated under reduced pressure to get the residue, which was then purified by column chromatography using 40% ethyl acetate in hexane as an eluent to obtain 4-(5-(difluoromethyl)-1,2,4-oxadiazol-3-yl)-N-((4-fluorophenyl)(methyl)(oxo)-λ⁶-sulfaneylidene)benzamide (0.4 g, 81% yield).

### Step 4:—4-(5-(Difluoromethyl)-4,5-dihydro-1,2,4-oxadiazol-3-yl)-N-((4-fluorophenyl)(methyl)(oxo)-λ⁶-sulfaneylidene)benzamide

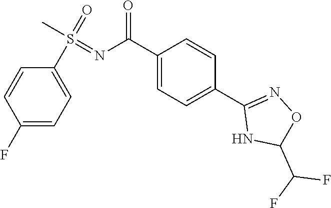 [SMILES: CS(=O)(=NC(=O)c1ccc(C2=NOC(C(F)F)N2)cc1)c1ccc(F)cc1]

To a solution of 4-(5-(difluoromethyl)-1,2,4-oxadiazol-3-yl)-N-((4-fluorophenyl)(methyl)(oxo)-λ⁶-sulfanylidene)benzamide (200 mg, 0.5 mmol) in tetrahydrofuran (2 mL) was added borane-dimethyl sulphide complex (0.2 mL, 1.5 mmol) at 0-5° C. then stirred at 25° C. for 2 h. After completion of the reaction, the reaction mixture was quenched by adding methanol (3 mL) drop wise at 0-5° C., added water (10 mL), extracted with ethyl acetate (15 mL). Ethyl acetate layer was dried over sodium sulphate, concentrated under reduced pressure to obtain crude solid, which was triturated with hexanes (15 mL), filtered, washed with hexane (5 mL) and dried under reduced pressure to afford 4-(5-(difluoromethyl)-4,5-dihydro-1,2,4-oxadiazol-3-yl)-N-((4-fluorophenyl)(methyl)(oxo)-λ⁶-sulfaneylidene)benzamide (150 mg, 75% yield).

### Example 18:—Preparation of 2-phenyl-N-(4-(5-(trifluoromethyl)-1h-1,2,4-triazol-3-yl)phenyl)propanamide (Compound 39)

### Step 1:—tert-butyl (4-cyanophenyl)carbamate

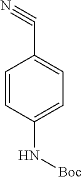 [SMILES: CC(C)(C)OC(=O)Nc1ccc(C#N)cc1]

To a stirred solution of 4-aminobenzonitrile (15 g, 127 mmol) in dichloromethane (10 mL) was added 4-dimethylaminopyridine (3.1 g, 25.4 mmol) and boc-anhydride (32.4 mL, 140 mmol) at 0-5° C. and further stirred at room temperature for 48 h. After completion of reaction, water (100 mL) was added and product was extracted using ethyl acetate (150 mL). The ethyl acetate layer was dried over anhydrous sodium sulfate and concentrated to obtain crude mass. This was further purified using column chromatography using 50% ethyl acetate in hexane as an eluent to get the pure compound tert-butyl (4-cyanophenyl)carbamate (10.5 g, 38% yield).

### Step 2:—tert-Butyl (4-(N′-hydroxycarbamimidoyl)phenyl)carbamate

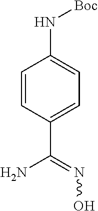 [SMILES: CC(C)(C)OC(=O)Nc1ccc(C(N)=NO)cc1]

A stirred suspension of tert-butyl (4-cyanophenyl)carbamate (9.5 g, 43.5 mmol), hydroxylamine hydrochloride (4.5 g, 65.3 mmol) and sodium bicarbonate (5.5 g, 65.3 mmol) in ethanol (50 mL) was refluxed for 16 h. After completion of reaction, the reaction mixture was concentrated under reduced pressure, the residue obtained was diluted with cold water (200 mL). The resulting precipitate was filtered off and washed with cold water (50 mL) to afford tert-butyl (4-(N′-hydroxycarbamimidoyl)phenyl)carbamate (9.2 g, 84% yield).

### Step 3:—tert-Butyl (4-(5-(trifluoromethyl)-1,2,4-oxadiazol-3-yl)phenyl)carbamate

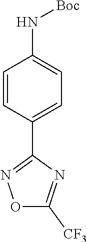 [SMILES: CC(C)(C)OC(=O)Nc1ccc(-c2noc(C(F)(F)F)n2)cc1]

To a stirred solution of tert-butyl (4-(N′-hydroxycarbamimidoyl)phenyl)carbamate (6.5 g, 25.9 mmol) in dry tetrahydrofuran (80 mL), trifluoroacetic anhydride (4.4 mL, 31.0 mmol) was added drop wise at 0-5° C. and stirred for 16 h at 25° C. After completion of the reaction, water was added and the reaction mixture was extracted with ethyl acetate (100 mL). The ethyl acetate layer was washed with saturated aqueous sodium bicarbonate (200 mL), dried over anhydrous sodium sulphate and concentrated under reduced pressure to get the residue. This residue was purified by column chromatography using 40% ethyl acetate in hexane as an eluent to afford tert-butyl (4-(5-(trifluoromethyl)-1,2,4-oxadiazol-3-yl)phenyl)carbamate (6.0 g, 70% yield).

### Step-4:—4-(5-(Trifluoromethyl)-1,2,4-oxadiazol-3-yl)aniline

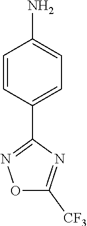 [SMILES: Nc1ccc(-c2noc(C(F)(F)F)n2)cc1]

To a stirred solution of tert-butyl (4-(5-(trifluoromethyl)-1,2,4-oxadiazol-3-yl)phenyl)carbamate (1.5 g, 4.5 mmol) in dichloromethane (15 mL) was added trifluoroacetic acid (2.0 mL, 26 mmol) at 0-5° C. and stirred for 2 h at 25° C. After completion of the reaction it was neutralized with saturated sodium bicarbonate solution and product was extracted by ethyl acetate (50 mL) and distilled to get 4-(5-(trifluoromethyl)-1,2,4-oxadiazol-3-yl)aniline (0.8 g, 3.5 mmol, 77% yield).

### Step 5:—2-Phenyl-N-(4-(5-(trifluoromethyl)-1,2,4-oxadiazol-3-yl)phenyl)propanamide

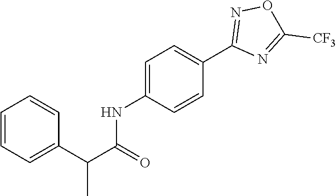 [SMILES: CC(C(=O)Nc1ccc(-c2noc(C(F)(F)F)n2)cc1)c1ccccc1]

To a stirred solution of 2-phenylpropanoic acid (0.1 g, 0.7 mmol) in N,N-dimethylformamide (3 mL) was added 1-[bis(dimethylamino)methylene]-1H-1,2,3-triazolo[4,5-b]pyridinium 3-oxide hexafluorophosphate (0.304 g, 0.8 mmol) and stirred for 15 minute at 0-5° C. followed by addition of 4-(5-(trifluoromethyl)-1,2,4-oxadiazol-3-yl)aniline (0.168 g, 0.732 mmol) and N,N-diisopropylethylamine (0.291 mL, 1.67 mmol) and stirred at 25° C. for 2 h. After completion of the reaction, water (100 mL) was added and product was extracted thrice by ethyl acetate (50 mL). The combined ethyl acetate layers were dried over anhydrous sodium sulfate and concentrated to get crude residue which was further purified by column chromatography using 60% ethyl acetate in hexane as an eluent to obtain 2-phenyl-N-(4-(5-(trifluoromethyl)-1,2,4-oxadiazol-3-yl)phenyl)propanamide (145 mg, 60% yield).

### Step 6:—2-Phenyl-N-(4-(5-(trifluoromethyl)-1h-1,2,4-triazol-3-yl)phenylpropanamide

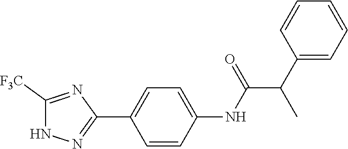 [SMILES: CC(C(=O)Nc1ccc(-c2n[nH]c(C(F)(F)F)n2)cc1)c1ccccc1]

To a stirred solution of 2-phenyl-N-(4-(5-(trifluoromethyl)-1,2,4-oxadiazol-3-yl)phenyl)propanimide (0.3 g, 0.7 mmol) in ethanol (10 mL) was added hydrazine hydrate (0.07 mL, 2.1 mmol) at 0-5° C. The reaction mixture was stirred at 65° C. for 16 h. After completion of reaction, the reaction mixture was concentrated under reduced pressure to get a crude compound which was purified by column chromatography using 50% ethyl acetate in hexane as an eluent to get 2-phenyl-N-(4-(5-(trifluoromethyl)-1h-1,2,4-triazol-3-yl)phenyl)propanamide (105 mg, 42% yield).

TABLE 8
The following compound was prepared by procedure analogous to that of the
compound 39.

| Compound | | | |
|---|---|---|---|
| no. | Structure | Iupac name | Yield |
| 36 | 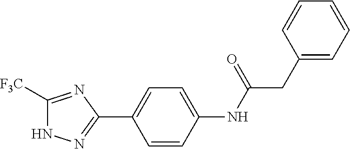 [SMILES: O=C(Cc1ccccc1)Nc1ccc(-c2n[nH]c(C(F)(F)F)n2)cc1] | 2-phenyl-N-(4-(5- (trifluoromethyl)-1h-1,2,4- triazol-3- yl)phenyl)acetamide | 0.06 g, 24% yield |

### Example 19:—Preparation of 2-phenyl-N-(4-(5-(trifluoromethyl)-4,5-dihydro-1,2,4-oxadiazol-3-yl)phenyl)propanamide (Compound 20)

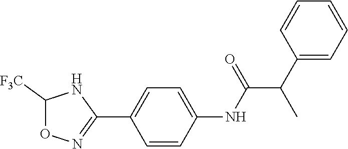 [SMILES: CC(C(=O)Nc1ccc(C2=NOC(C(F)(F)F)N2)cc1)c1ccccc1]

2-Phenyl-N-(4-(5-(trifluoromethyl)-4,5-dihydro-1,2,4-oxadiazol-3-yl)phenyl)propanamide (0.1 g, 41% yield) and compound no. 23 were prepared by the procedure analogous to that of the compound 27.

| Compound | | | |
|---|---|---|---|
| no. | Structure | Iupac name | Yield |
| 23 | 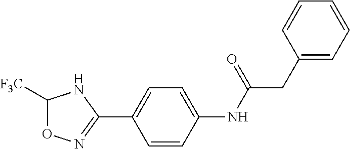 [SMILES: O=C(Cc1ccccc1)Nc1ccc(C2=NOC(C(F)(F)F)N2)cc1] | 2-phenyl-N-(4-(5- (trifluoromethyl)-4,5- dihydro-1,2,4-oxadiazol-3- yl)phenyl)acetamide | 0.09 g, 36% yield |

### Example 20:—Preparation of N-(4-(5-(difluoromethyl)-1,2,4-oxadiazol-3-yl)phenyl)-2-phenylacetamide (Compound 31)

### Step 1: tert-Butyl (4-(5-(difluoromethyl)-1,2,4-oxadiazol-3-yl)phenyl)carbamate

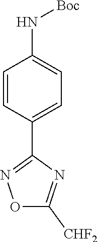 [SMILES: CC(C)(C)OC(=O)Nc1ccc(-c2noc(C(F)F)n2)cc1]

To a stirred solution of tert-butyl (4-(N′-hydroxycarbamimidoyl)phenyl)carbaminate (6.5 g, 26 mmol) in dry tetrahydrofuran (10 mL) 2,2-difluoroacetic anhydride (5.4 g, 31.0 mmol) was added drop wise at 0-5° C. and stirred for 16 h at 25° C. After completion of the reaction, the reaction mixture was extracted with ethyl acetate (500 mL). Ethyl acetate layer was washed with saturated sodium bicarbonate solution (300 mL), dried over sodium sulphate and concentrated under reduced pressure to obtain a crude product. This was purified by column chromatography using 60% ethyl acetate in hexane to obtain tert-butyl (4-(5-(difluoromethyl)-1,2,4-oxadiazol-3-yl)phenyl)carbamate (5.2 g, 65% yield).

### Step 2:—4-(5-(Difluoromethyl)-1,2,4-oxadiazol-3-yl)aniline

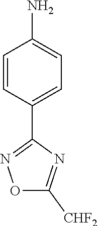 [SMILES: Nc1ccc(-c2noc(C(F)F)n2)cc1]

To a stirred solution of tert-butyl (4-(5-(difluoromethyl)-1,2,4-oxadiazol-3-yl)phenyl)carbamate (2.2 g, 7.1 mmol) in dichloromethane (25 mL) was added trifluoroacetic acid (2.0 mL, 26.0 mmol) at 0-5° C. and stirred for 2 h at 25° C. After completion of the reaction it was neutralized with aqueous saturated sodium bicarbonate solution (30 mL) and the product was extracted with ethyl acetate (60 mL). The ethyl acetate layer was dried over anhydrous sodium sulphate and concentrated under reduced pressure to obtain 4-(5-(difluoromethyl)-1,2,4-oxadiazol-3-yl)aniline (1.3 g, 87% yield).

### Step 3:—N-(4-(5-(difluoromethyl)-1,2,4-oxadiazol-3-yl)phenyl)-2-phenylacetamide

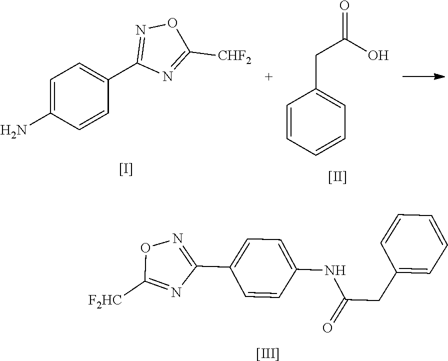

To a stirred solution of 2-phenylacetic acid (0.25 g 1.8 mmol) in N,N-dimethylformamide (3 mL) was added 1-[bis(dimethylamino)methylene]-1H-1,2,3-triazolo[4,5-b]pyridinium 3-oxide hexafluorophosphate (0.84 g, 2.2 mmol) and stirred for 15 min at 0-5° C. followed by addition of 4-(5-(difluoromethyl)-1,2,4-oxadiazol-3-yl)aniline (0.4 g, 1.8 mmol) and diisopropylethylamine (0.8 mL, 4.6 mmol) and stirred at 25° C. for 2-3 h. After completion of the reaction, water (10 mL) was added and the product was extracted using ethyl acetate (30 mL). The ethyl acetate layer was dried over anhydrous sodium sulfate and concentrated under reduced pressure to get crude mass which was purified by column chromatography using 50% ethyl acetate in hexane as an eluent to obtain N-(4-(5-(difluoromethyl)-1,2,4-oxadiazol-3-yl)phenyl)-2-phenylacetamide (177 mg, 29% yield).

### Example 21:—Preparation of N-methyl-N-(4-(5-(trifluoromethyl)-4,5-dihydro-1,2,4-oxadiazol-3-yl)benzyl)cyclopropanecarboxamide (Compound 21)

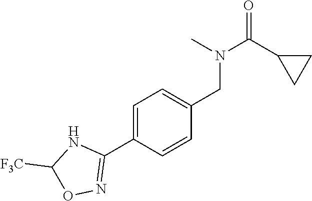 [SMILES: CN(Cc1ccc(C2=NOC(C(F)(F)F)N2)cc1)C(=O)C1CC1]

### Step 1:—N-methyl-1-(4-(5-(trifluoromethyl)-1,2,4-oxadiazol-3-yl)phenyl)methanamine

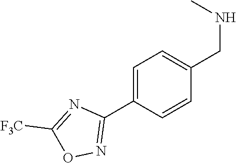 [SMILES: CNCc1ccc(-c2noc(C(F)(F)F)n2)cc1]

To a solution of 3-(4-(bromomethyl)phenyl)-5-(trifluoromethyl)-1,2,4-oxadiazole (0.7 g, 2.3 mmol) and N,N-dimethylformamide (5 mL) was added 2 M methylamine in tetrahydrofuran (6.8 mL, 13.7 mmol) at 25° C. and stirred for 16 h. The reaction mixture was extracted with dichloromethane (30 mL). The dichloromethane layer was washed twice with saturated sodium bicarbonate solution (10 mL), dried over anhydrous sodium sulphate and concentrated under reduced pressure. Crude product obtained as purified by column chromatography using eluent 10% methanol in dichloromethane as an eluent (0.3 g, 43%)

### Step 2:—N-methyl-N-(4-(5-(trifluoromethyl)-1,2,4-oxadiazol-3-yl)benzyl)cyclopropanecarboxamide

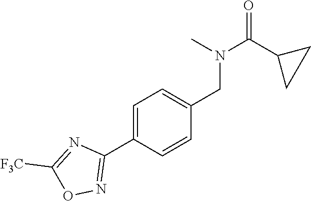 [SMILES: CN(Cc1ccc(-c2noc(C(F)(F)F)n2)cc1)C(=O)C1CC1]

To a solution of N-methyl-1-(4-(5-(trifluoromethyl)-1,2,4-oxadiazol-3-yl)phenyl)methanamine (1.5 g, 5.8 mmol), triethylamine (3.3 mL, 23.3 mmol) and dichloromethane (10 mL) was added cyclopropanecarbonyl chloride (0.5 mL, 5.8 mmol) at 5° C. and stirred at 25° C. for 4 h. After completion of reaction, the reaction mixture was extracted with dichloromethane (30 mL). The dichloromethane layer was washed twice with saturated sodium bicarbonate solution, dried over anhydrous sodium sulphate and concentrated under reduced pressure. The crude product obtained was purified by column chromatography using 50% ethyl acetate in hexane as an eluent to get N-methyl-N-(4-(5-(trifluoromethyl)-1,2,4-oxadiazol-3-yl)benzyl)cyclopropanecarboxamide (1.2 g, 3.7 mmol, 63% yield).

### Step-3N-methyl-N-(4-(5-(trifluoromethyl)-4,5-dihydro-1,2,4-oxadiazol-3-yl)benzyl)cyclopropanecarboxamide

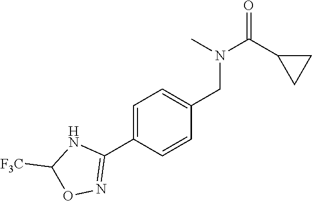 [SMILES: CN(Cc1ccc(C2=NOC(C(F)(F)F)N2)cc1)C(=O)C1CC1]

To a solution of N-methyl-N-(4-(5-(trifluoromethyl)-1,2,4-oxadiazol-3-yl)benzyl)cyclopropanecarboxamide (0.3 g, 0.8 mmol) in tetrahydrofuran (3 mL) was added borane-methyl sulfide complex (0.2 mL, 2.3 mmol) at −15° C. and stirred at 0° C. for 30 minutes and at 25° C. for 30 minutes. After completion of reaction, the reaction mixture was quenched by drop wise addition of methanol (5 mL). Reaction mixture was concentrated and obtained residue was purified by column chromatography using 50% ethyl acetate in hexane as an eluent (0.135 g, 54% yield).

TABLE 9
Compound 22 was prepared by the procedure analogous to that of the compound 21.
Compound 37 and 38 were prepared by procedure analogous to that of 27.

| Compound | | | |
|---|---|---|---|
| no. | Structure | Iupac name | Yield |
| 22 | 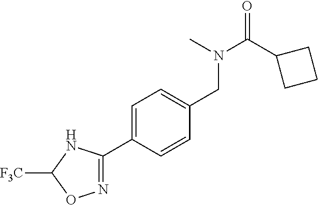 [SMILES: CN(Cc1ccc(C2=NOC(C(F)(F)F)N2)cc1)C(=O)C1CCC1] | N-methyl-N-(4-(5-(trifluoromethyl)- 4,5-dihydro-1,2,4-oxadiazol-3- yl)benzyl)cyclobutanecarboxamide | 0.172 g, 68% yield |
| 37 | 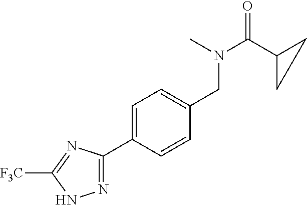 [SMILES: CN(Cc1ccc(-c2n[nH]c(C(F)(F)F)n2)cc1)C(=O)C1CC1] | N-methyl-N-(4-(5-(trifluoromethyl)- 1h-1,2,4-triazol-3- yl)benzyl)cyclopropanecarboxamide | 0.132 g, 44% |
| 38 | 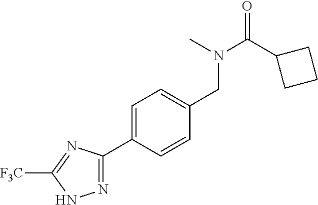 [SMILES: CN(Cc1ccc(-c2n[nH]c(C(F)(F)F)n2)cc1)C(=O)C1CCC1] | N-methyl-N-(4-(5-(trifluoromethyl)- 1h-1,2,4-triazol-3- yl)benzyl)cyclobutanecarboxamide | 0.1 g, 37% |

### Example 22:—Preparation of N-methyl-4-(5-(perfluoroethyl)-1,2,4-oxadiazol-3-yl)-N-(2-phenoxyethyl)benzamide (Compound 53)

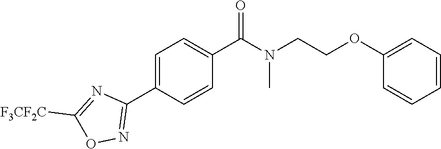

To a stirred solution of 4-(N′-hydroxycarbamimidoyl)-N-methyl-N-(2-phenoxyethyl)benzamide (0.6 g, 1.9 mmol) in tetrahydrofuran (30 mL) was added difluoroacetic anhydride (03 mL, 2.6 mmol) at 0° C. under nitrogen atmosphere. The reaction mixture was allowed to stir at 25° C. for 16 h. After completion of the reaction, the reaction mixture was diluted with ethyl acetate (40 mL) and washed with ice cold sodium bicarbonate solution (40 ml). The organic layer was dried over anhydrous sodium sulphate. The solvent was evaporated under reduced pressure to get crude residue which was purified by column chromatography by using 60% ethyl acetate in hexane to obtain N-methyl-4-(5-(perfluoroethyl)-1,2,4-oxadiazol-3-yl)-N-(2-phenoxyethyl)benzamide (380 mg, 43% yield)

### Example 23:—Preparation of N-(2,4-difluorophenyl)-4-(5-(trifluoromethyl)isoxazol-3-yl)benzamide (Compound 57)

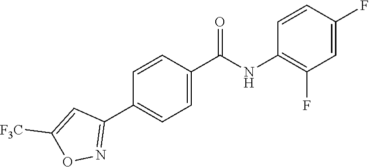 [SMILES: O=C(Nc1ccc(F)cc1F)c1ccc(-c2cc(C(F)(F)F)on2)cc1]

### Step 1:—Synthesis of ethyl-4-(4,4,4-trifluoro-3-oxobutanoyl)benzoate

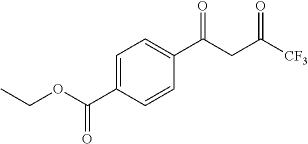 [SMILES: CCOC(=O)c1ccc(C(=O)CC(=O)C(F)(F)F)cc1]

To a suspension of sodium ethoxide (0.4 g, 6.2 mmol) in toluene (10 mL), ethyl-2,2,2-trifluoroacetate (0.6 g, 4.2 mmol) was added and allowed to stir at 0° C. for 30 minutes. Methyl-4-acetylbenzoate (0.5 g, 2.81 mmol) was added portion wise. The reaction was allowed to stir at 25° C. for further 16 h. The reaction was then cooled to 0-5° C. and filtered. The filtered solid was dissolved in ethyl acetate (30 mL) and washed with aqueous 5% aqueous sulphuric acid (20 mL). The ethyl acetate layer was washed twice with brine solution (10 mL), dried over anhydrous magnesium sulphate and concentrated under reduced pressure to obtain ethyl-4-(4,4,4-trifluoro-3-oxobutanoyl)benzoate (0.7 g, 80% yield).

### Step 2:—Ethyl-4-(5-hydroxy-5-(trifluoromethyl)-4,5-dihydroisoxazol-3-yl)benzoate-2,2,2-trifluoroacetaldehyde

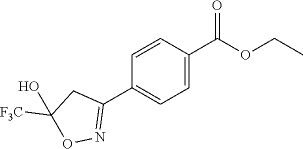 [SMILES: CCOC(=O)c1ccc(C2=NOC(O)(C(F)(F)F)C2)cc1]

A solution of ethyl-4-(4,4,4-trifluoro-3-oxobutanoyl)benzoate (0.7 g, 2.3 mmol) in glacial acetic acid (1.5 mL) was treated with hydroxylamine hydrochloride (0.2 g, 2.7 mmol) and heated at 80-90° C. for 4 h. The reaction was cooled to 25° C. and the resulting solid was filtered and washed with water (10 mL) to obtain ethyl-4-(5-hydroxy-5-(trifluoromethyl)-4,5-dihydroisoxazol-3-yl)benzoate (0.35 g, 51% yield).

### Step 3:—Ethyl-4-(5-(trifluoromethyl)isoxazol-3-yl)benzoate

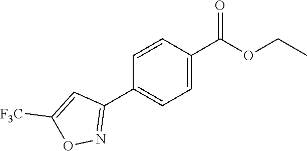 [SMILES: CCOC(=O)c1ccc(-c2cc(C(F)(F)F)on2)cc1]

A solution of ethyl-4-(5-hydroxy-5-(trifluoromethyl)-4,5-dihydroisoxazol-3-yl)benzoate (0.4 g, 1.2 mmol) in trifluoroacetic acid (2.5 mL) was stirred at 100-110° C. for 24 h. The reaction mixture was cooled to 25° C., the compound was extracted with ethyl acetate (20 mL) and washed with aqueous saturated potassium carbonate solution (10 mL). Ethyl acetate layer was further washed with brine solution (5 mL), dried over anhydrous sodium sulphate, filtered and evaporated under reduced pressure to obtain a crude product. The crude product was purified by column chromatography using 40% ethyl acetate in hexane as an eluent to obtain ethyl-4-(5-(trifluoromethyl)isoxazol-3-yl)benzoate (0.16 g, 49% yield).

### Step 4:—4-(5-(Trifluoromethyl)isoxazol-3-yl)benzoic Acid

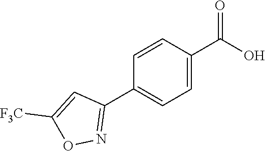 [SMILES: O=C(O)c1ccc(-c2cc(C(F)(F)F)on2)cc1]

To a solution of ethyl-4-(5-(trifluoromethyl)isoxazol-3-yl)benzoate (0.5 g, 1.7 mmol) in tetrahydrofuran (10 mL), aqueous solution (3 mL) of lithium hydroxide hydrate (0.2 g, 3.5 mmol) was added and allowed to stir for 16 h. After completion of reaction, the reaction was then evaporated to and treated with a 1 N aqueous hydrochloric acid (15 mL). The solid obtained was filtered, washed with water (5 mL) and diethyl ether (5 mL) and dried to obtain 4-(5-(trifluoromethyl)isoxazol-3-yl)benzoic acid (0.33 g, 73% yield).

### Step 5:—N-(2,4-difluorophenyl)-4-(5-(trifluoromethyl)isoxazol-3-yl)benzamide

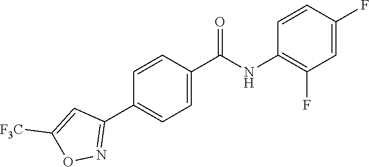 [SMILES: O=C(Nc1ccc(F)cc1F)c1ccc(-c2cc(C(F)(F)F)on2)cc1]

To a solution of 4-(5-(trifluoromethyl)isoxazol-3-yl)benzoic acid (0.2 g, 0.8 mmol) in dichloromethane (10 mL), 1-[Bis(dimethylamino)methylene]-1H-1,2,3-triazolo[4,5-b]pyridinium 3-oxide hexafluorophosphate (0.5 g, 1.2 mmol), triethylamine (0.3 mL, 2.1 mmol) and 2,4-difluoroaniline (0.13 mL, 1.3 mmol) were added at 0-5° C. under nitrogen atmosphere and stirred at 25° C. for 18 h. After completion of reaction, dichloromethane (20 mL) was added to the reaction mixture. Dichloromethane layer was washed with sodium bicarbonate solution (10 mL) and water (10 mL) then dried over anhydrous magnesium sulphate. The organic layer was evaporated under reduced pressure to get a crude product which was purified by column chromatography using 60% ethyl acetate in hexane as an eluent to obtain N-(2,4-difluorophenyl)-4-(5-(trifluoromethyl)isoxazol-3-yl)benzamide (0.23 g, 73% yield).

TABLE 10
The following compounds were prepared by the procedure analogous to that of the
compound 57.

| Compound | | | |
|---|---|---|---|
| no. | Structure | Iupac name | Yield |
| 64 | 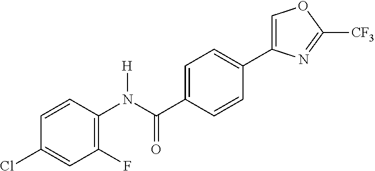 [SMILES: O=C(Nc1ccc(Cl)cc1F)c1ccc(-c2coc(C(F)(F)F)n2)cc1] | N-(4-chloro-2-fluorophenyl)- 4-(2-(trifluoromethyl)oxazol- 4-yl)benzamide | 210 mg, 49% yield |
| 65 | 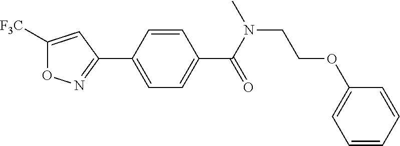 [SMILES: CN(CCOc1ccccc1)C(=O)c1ccc(-c2cc(C(F)(F)F)on2)cc1] | N-methyl-N-(2- phenoxyethyl)-4-(5- (trifluoromethyl)isoxazol-3- yl)benzamide | 0.170 g, 51% yield |

### Example 24:—Preparation of N-((4-fluorophenyl)(methyl)(oxo)-λ⁶-sulfaneylidene)-4-(5-(trifluoromethyl)isoxazol-3-yl)benzamide (Compound 58)

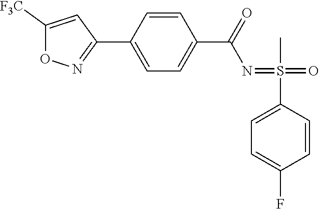 [SMILES: CS(=O)(=NC(=O)c1ccc(-c2cc(C(F)(F)F)on2)cc1)c1ccc(F)cc1]

To a solution of 4-(5-(trifluoromethyl)isoxazol-3-yl)benzoic acid (0.2 g, 0.8 mmol) in dichloromethane (8 mL), 4-dimethylaminopyridine (0.3 g, 2.6 mmol), 1-(3-dimethylaminopropyl)-3-ethylcarbodiimide hydrochloride (0.3 g, 1.7 mmol) and (4-fluorophenyl)(imino)(methyl)-λ⁶-sulfanone (0.2 g, 1.3 mmol) were added at 0-5° C. under nitrogen atmosphere and stirred at 25° C. for 18 h. After completion of reaction, dichloromethane (20 mL) was added to the reaction mixture. Dichloromethane layer was washed with sodium bicarbonate solution (10 mL) and water (10 mL) then dried over anhydrous magnesium sulphate and concentrated under reduced pressure. The crude compound was purified by column chromatography using 60% ethyl acetate in hexane as an eluent to obtain N-((4-fluorophenyl)(methyl)(oxo)-λ⁶-sulfanylidene)-4-(5-(trifluoromethyl)isoxazol-3-yl)benzamide (0.3 g, 0.7 mmol, 79% yield.

TABLE 11
The following compounds were prepared by the procedure analogous to that of the
compound 58.

| Compound | | | |
|---|---|---|---|
| no. | Structure | Iupac name | Yield |
| 59 | 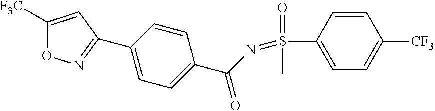 [SMILES: CS(=O)(=NC(=O)c1ccc(-c2cc(C(F)(F)F)on2)cc1)c1ccc(C(F)(F)F)cc1] | N-(methyl(oxo)(4- (trifluoromethyl)phenyl)-λ⁶- sulfaneylidene)-4-(5- (trifluoromethyl)isoxazol-3- yl)benzamide | 0.302 g, 76% yield |
| 60 | 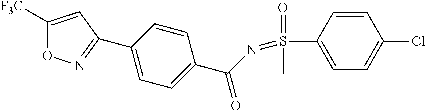 [SMILES: CS(=O)(=NC(=O)c1ccc(-c2cc(C(F)(F)F)on2)cc1)c1ccc(Cl)cc1] | N-((4- chlorophenyl)(methyl)(oxo)- λ⁶-sulfaneylidene)-4-(5- (trifluoromethyl)ixoazol-3- yl)benzamide | 0.125 g, 34% yield |
| 61 | 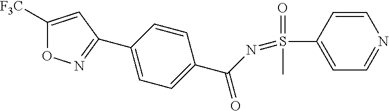 [SMILES: CS(=O)(=NC(=O)c1ccc(-c2cc(C(F)(F)F)on2)cc1)c1ccncc1] | N-(methyl(oxo)(pyridiN-4-yl)- λ⁶-sulfaneylidene)-4-(5- (trifluoromethyl)isoxazol-3- yl)benzamide | 0.148 g, 44% yield |
| 62 | 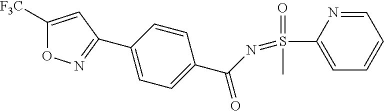 [SMILES: CS(=O)(=NC(=O)c1ccc(-c2cc(C(F)(F)F)on2)cc1)c1ccccn1] | N-(methyl(oxo)(pyridin-2-yl)- λ⁶-sulfaneylidene)-4-(5- (trifluoromethyl)isoxazol-3- yl)benzamide | 0.350 g, 76% yield |
| 63 | 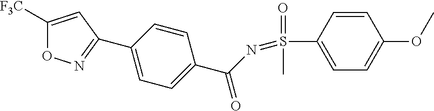 [SMILES: COc1ccc(S(C)(=O)=NC(=O)c2ccc(-c3cc(C(F)(F)F)on3)cc2)cc1] | N-((4- methoxyphenyl)(methyl)(oxo)- λ⁶-sulfaneylidene)-4-(5- (trifluoromethyl)isoxazol-3- yl)benzamide | 0.265 g, 64% yield |
| 66 | 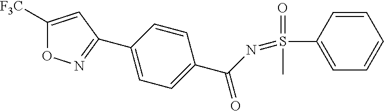 [SMILES: CS(=O)(=NC(=O)c1ccc(-c2cc(C(F)(F)F)on2)cc1)c1ccccc1] | N-(methyl(oxo)(phenyl)-λ⁶- sulfaneylidene)-4-(5- (trifluoromethyl)isoxazol-3- yl)benzamide | 0.375 g, 82% yield |

### Example 25:—Preparation of N-methyl-N-(2-phenoxyethyl)-4-(2-(trifluoromethyl)oxazol-4-yl)benzamide (Compound 67)

### Step 1:—methyl 4-(1-(hydroxyimino)ethyl)benzoate

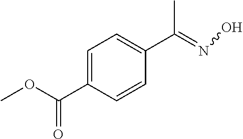

To a solution of methyl 4-acetylbenzoate (10 g, 56.1 mmol) in methanol (100 mL) was added sodium acetate (9.2 g, 112 mmol), hydroxylamine hydrochloride (4.7 g, 67.3 mmol) and stirred at 25° C. for 16 h. Reaction mixture was diluted with 400 mL of water, stirred for 30 min. The solid obtained was filtered and dried under reduced pressure to obtain methyl 4-(1-(hydroxyimino)ethyl)benzoate (10 g, 92% yield).

### Step 2:—Methyl 4-(2-iodo-1-(2,2,2-trifluoroacetamido)vinyl)benzoate

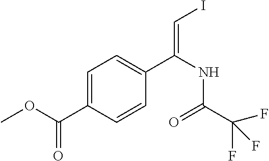 [SMILES: COC(=O)c1ccc(/C(=C/I)NC(=O)C(F)(F)F)cc1]

To a solution of methyl 4-(1-(hydroxyimino)ethyl)benzoate (11 g, 56.9 mmol) in 1,2-dichloromethane (160 mL) was added trifluoroacetic anhydride (23.7 mL, 171 mmol), cuprous iodide (32.5 g, 171 mmol) then heated at 80° C. for 16 h. The reaction mixture was quenched by pouring onto ice-cold water (200 mL) and extracted with ethyl acetate (200 mL). Organic layer was washed with water (50 mL), brine solution (50 mL) and dried over anhydrous sodium sulphate and concentrated under reduced pressure to obtain a crude product. The crude product was purified by column chromatography using 30% ethyl acetate in hexane to obtain methyl 4-(2-iodo-1-(2,2,2-trifluoroacetamido)vinyl)benzoate (12 g, 54%).

### Step 3: Methyl 4-(2-(trifluoromethyl)oxazol-4-yl)benzoate

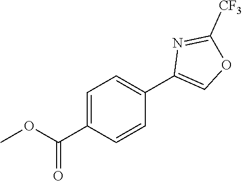 [SMILES: COC(=O)c1ccc(-c2coc(C(F)(F)F)n2)cc1]

To a solution of methyl 4-(2-iodo-1-(2,2,2-trifluoroacetamido)vinyl)benzoate (12 g, 30 mmol) in N,N-dimethylformamide (120 mL) was added cuprous iodide (0.6 g, 3 mmol) and potassium carbonate (8.3 g, 60 mmol) then heated at 80° C. for 3 h. The reaction mixture was diluted with water (100 mL), extracted with ethyl acetate (300 mL). Ethyl acetate layer was washed with water (200 mL), brine solution (100 mL), dried over anhydrous sodium sulphate and concentrated under reduced pressure to obtain a crude product. Purification was done by column chromatography using 20% ethyl acetate in hexane to obtain methyl 4-(2-(trifluoromethyl)oxazol-4-yl)benzoate (2 g, 25% yield).

### Step 4:—N-methyl-N-(2-phenoxyethyl)-4-(2-(trifluoromethyl)oxazol-4-yl)benzamide

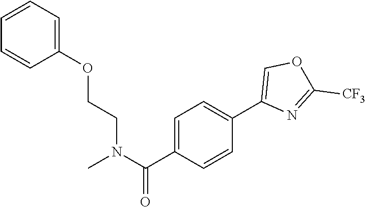 [SMILES: CN(CCOc1ccccc1)C(=O)c1ccc(-c2coc(C(F)(F)F)n2)cc1]

To a stirred solution of methyl 4-(2-(trifluoromethyl)oxazol-4-yl)benzoate (400 mg, 1.5 mmol) in toluene was added N-methyl-2-phenoxyethan-1-amine (335 mg, 2.2 mmol) and trimethylaluminium (1475 μl, 2.9 mmol) at 0-5° C. and then stirred at 80° C. for 24 h. After completion of reaction, the reaction was quenched by pouring onto ice cold 10% aqueous acetic acid, extracted with ethyl acetate (50 mL). The ethyl acetate layer was washed with water (10 mL), brine solution (10 mL), dried over anhydrous sodium sulphate and concentrated under reduced pressure to afford crude product, The crude product was purified by column chromatography using 60% ethyl acetate in hexane as an eluent to obtain N-methyl-N-(2-phenoxyethyl)-4-(2-(trifluoromethyl)oxazol-4-yl)benzamide (0.5 g, 85% yield).

TABLE 12
The following compounds were prepared by the procedure analogous to that of the
compound 67.

| Compound | | | |
|---|---|---|---|
| no. | Structure | Iupac name | Yield |
| 77 | 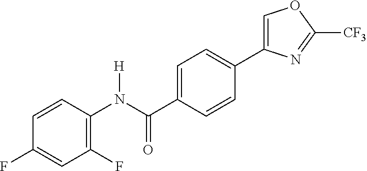 [SMILES: O=C(Nc1ccc(F)cc1F)c1ccc(-c2coc(C(F)(F)F)n2)cc1] | N-(2,4-difluorophenyl)-4-(2- (trifluoromethyl)oxazol-4- yl)benzamide | 175 mg, 43% yield |
| 80 | 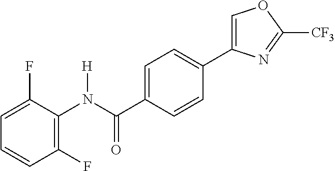 [SMILES: O=C(Nc1c(F)cccc1F)c1ccc(-c2coc(C(F)(F)F)n2)cc1] | N-(2,6-difluorophenyl)-4-(2- (trifluoromethyl)oxazol-4- yl)benzamide | 175 mg, 43% yield |
| 81 | 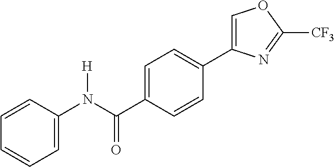 [SMILES: O=C(Nc1ccccc1)c1ccc(-c2coc(C(F)(F)F)n2)cc1] | N-phenyl-4-(2- (trifluoromethyl)oxazol-4- yl)benzamide | 180 mg, 49% yield |
| 82 | 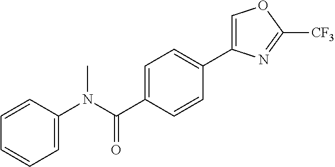 [SMILES: CN(C(=O)c1ccc(-c2coc(C(F)(F)F)n2)cc1)c1ccccc1] | N-methyl-N-phenyl-4-(2- (trifluoromethyl)oxazol-4- yl)benzamide | 155 mg, 41% yield |

### Example 26:—Preparation of N-methyl(oxo)(pyridin-4-yl)-λ⁶-sulfaneylidene)-4-(2-(trifluoromethyl)oxazol-4-yl)benzamide (Compound 68)

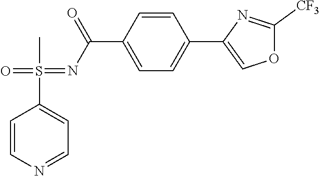 [SMILES: CS(=O)(=NC(=O)c1ccc(-c2coc(C(F)(F)F)n2)cc1)c1ccncc1]

To a solution of methyl 4-(2-(trifluoromethyl)oxazol-4-yl)benzoate (191 mg, 0.7 mmol) in toluene (2 mL) was added imino(methyl)(pyridin-4-yl)-λ⁶-sulfanone (100 mg, 0.6 mmol) then added trimethylaluminium, 2M in toluene (46.1 mg, 0.6 mmol) at 0-5° C. and then stirred at 80° C. for 24 h. After completion of reaction, the reaction was quenched by pouring onto ice cold 10% aqueous acetic acid (10 mL) and extracted with ethyl acetate (50 mL). The ethyl acetate layer was washed with water (10 mL), brine solution (10 mL), dried over anhydrous sodium sulphate and concentrated under reduced pressure to afford crude product. The crude product was purified by column chromatography using 60% ethyl acetate in hexane to obtain N-(methyl(oxo)(pyridin-4-yl)-λ⁶-sulfaneylidene)-4-(2-(trifluoromethyl)oxazol-4-yl)benzamide (135 mg, 53% yield).

TABLE 13
The following compounds were prepared by the procedure analogous to that of the
compound 68.

| Compound | | | |
|---|---|---|---|
| no. | Structure | Iupac name | Yield |
| 69 | 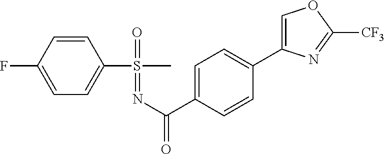 [SMILES: CS(=O)(=NC(=O)c1ccc(-c2coc(C(F)(F)F)n2)cc1)c1ccc(F)cc1] | N-((4- fluorophenyl)(methyl)(oxo)-λ⁶- sulfaneylidene)-4-(2- (trifluoromethyl)oxazol-4- yl)benzamide | 0.125 g, 41% yield |
| 72 | 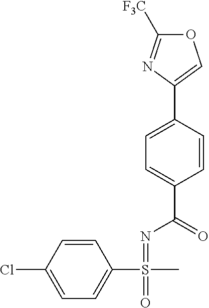 [SMILES: CS(=O)(=NC(=O)c1ccc(-c2coc(C(F)(F)F)n2)cc1)c1ccc(Cl)cc1] | N-((4- chlorophenyl)(methyl)(oxo)- λ⁶-sulfaneylidene)-4-(2- (trifluoromethyl)oxazol-4- yl)benzamide | 0.21 g, 66% yield |
| 78 | 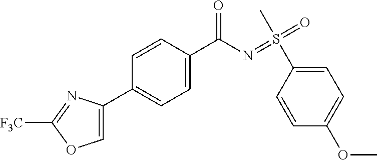 [SMILES: COc1ccc(S(C)(=O)=NC(=O)c2ccc(-c3coc(C(F)(F)F)n3)cc2)cc1] | N-((4- methoxyphenyl)(methyl)(oxo)- λ⁶-sulfaneylidene)-4-(2- (trifluoromethyl)oxazol-4- yl)benzamide | 350 mg, 36% yield |
| 83 | 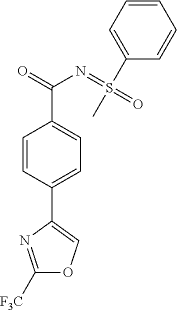 [SMILES: CS(=O)(=NC(=O)c1ccc(-c2coc(C(F)(F)F)n2)cc1)c1ccccc1] | N-(methyl(oxo)(phenyl)-λ⁶- sulfaneylidene)-4-(2- (trifluoromethyl)oxazol-4- yl)benzamide | 172 mg, 46% yield |
| 84 | 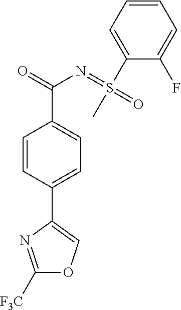 [SMILES: CS(=O)(=NC(=O)c1ccc(-c2coc(C(F)(F)F)n2)cc1)c1ccccc1F] | N-((2- fluorophenyl)(methyl)(oxo)-λ⁶- sulfaneylidene)-4-(2- (trifluoromethyl)oxazol-4- yl)benzamide | 175 mg, 46% yield |

### Example 27 Preparation of 4-(5-chloro-2-(trifluoromethyl)oxazol-4-yl)-N-(2,4-difluorophenyl)benzamide (Compound 70)

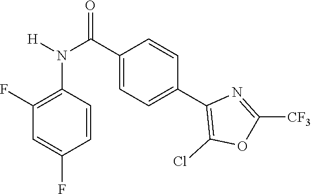 [SMILES: O=C(Nc1ccc(F)cc1F)c1ccc(-c2nc(C(F)(F)F)oc2Cl)cc1]

### Step 1:—Methyl 4-(5-chloro-2-(trifluoromethyl)oxazol-4-yl)benzoate

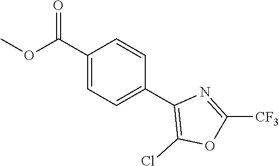 [SMILES: COC(=O)c1ccc(-c2nc(C(F)(F)F)oc2Cl)cc1]

To a solution of methyl 4-(2-(trifluoromethyl)oxazol-4-yl)benzoate (1.5 g, 5.5 mmol) in acetonitrile (15 mL), was added N-chlorosuccinimide (3.7 g, 27.7 mmol) and stirred at 80° C. for 16 b. After completion of reaction, the product was extracted with ethyl acetate (50 mL). The ethyl acetate layer was washed with saturated sodium bicarbonate solution (10 mL), brine solution (10 mL), dried over anhydrous sodium sulphate and concentrated under reduced pressure to afford the crude product. The crude product was purified by column chromatography using 10% ethyl acetate in hexane as an eluent to obtain methyl 4-(5-chloro-2-(trifluoromethyl)oxazol-4-yl)benzoate (1.2 g, 71% yield).

### Step:—4-(5-Chloro-2-(trifluoromethyl)oxazol-4-yl)-N-(2,4-difluorophenyl)benzamide

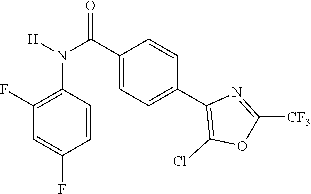 [SMILES: O=C(Nc1ccc(F)cc1F)c1ccc(-c2nc(C(F)(F)F)oc2Cl)cc1]

To a solution of methyl 4-(5-chloro-2-(trifluoromethyl)oxazol-4-yl)benzoate 200 mg, 0.7 mmol) in toluene (5 mL) was added 24-difluoroaniline (169 mg, 1.3 mmol) and trimethylaluminium (654 μl, 1.3 mmol) at 0-5° C. and then stirred at 80° C. for 24 h. After completion of reaction, the reaction was quenched by pouring onto ice cold 10% aqueous acetic acid (10 ml) and extracted with ethyl acetate (50 mL). The ethyl acetate layer was washed with water (10 mL), brine solution (10 mL), dried over anhydrous sodium sulphate and concentrated under reduced pressure to afford the crude product. The crude product was purified by column chromatography using 60% ethyl acetate in hexane to obtain 4-(5-chloro-2-(trifluoromethyl)oxazol-4-yl)-N-(2,4-difluorophenyl)benzamide (0.2 g, 70% yield).

TABLE 14
The following compound was prepared by the procedure analogous to that of the
compound 70.

| Compound | | | |
|---|---|---|---|
| no. | Structure | Iupac name | Yield |
| 71 | 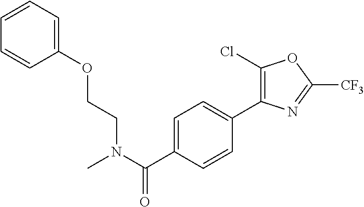 [SMILES: CN(CCOc1ccccc1)C(=O)c1ccc(-c2nc(C(F)(F)F)oc2Cl)cc1] | 4-(5-chloro-2- (trifluoromethyl)oxazol- 4-yl)-N-methyl-N-(2- phenoxyethyl)benzamide | 0.130 g, 47% yield |

### Example 28:—Preparation of 4-(5-chloro-2-(trifluoromethyl)oxazol-4-yl)-N-((4-fluorophenyl)(methyl)(oxo)-λ⁶-sulfaneylidene)benzamide (Compound 73)

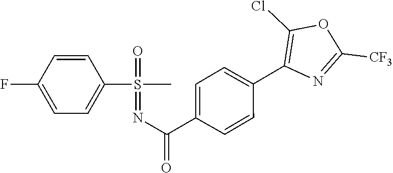 [SMILES: CS(=O)(=NC(=O)c1ccc(-c2nc(C(F)(F)F)oc2Cl)cc1)c1ccc(F)cc1]

To a solution of methyl 4-(5-chloro-2-(trifluoromethyl)oxazol-4-yl)benzoate (250 mg, 0.8 mmol) in toluene (5 mL) was added (4-fluorophenyl)(imino)(methyl)-λ⁶-sulfanone (213 mg, 1.2 mmol) and trimethylaluminium (818 μl, 1.6 mmol) at 0-5° C. and then heated at 80° C. for 24 h. After completion of reaction, the reaction was quenched by pouring onto ice cold 10% aqueous acetic acid (10 mL), extracted with ethyl acetate (50 mL). The ethyl acetate layer was washed with water (10 mL), brine solution (10 mL), dried over anhydrous sodium sulphate and concentrated under reduced pressure to afford the crude product. The crude product was purified by column chromatography using 60% ethyl acetate in hexane as an eluent to obtain 4-(5-chloro-2-(trifluoromethyl)oxazol-4-yl)-N-((4-fluorophenyl)(methyl)(oxo)-λ⁶-sulfaneylidene)benzamide (0.125 g, 45% yield)

TABLE 15
The following compounds were prepared by the procedure analogous to that of the
compound 73.

| Compound | | | |
|---|---|---|---|
| no. | Structure | Iupac name | Yield |
| 74 | 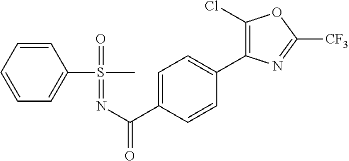 [SMILES: CS(=O)(=NC(=O)c1ccc(-c2nc(C(F)(F)F)oc2Cl)cc1)c1ccccc1] | 4-(5-chloro-2- (trifluoromethyl)oxazol-4-yl)- N-(methyl(oxo)(phenyl)-λ⁶- sulfaneylidene)benzamide | 0.185 g, 53% yield |
| 75 | 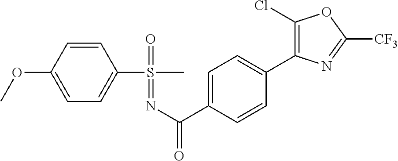 [SMILES: COc1ccc(S(C)(=O)=NC(=O)c2ccc(-c3nc(C(F)(F)F)oc3Cl)cc2)cc1] | 4-(5-chloro-2- (trifluoromethyl)oxazol-4-yl)- N-((4- methoxyphenyl)(methyl)(oxo)- λ⁶-sulfaneylidene)benzamide | 0.165 g, 44% yield |

### Example 29:—Preparation of 4-fluoro-N-(4-(5-(trifluoroethyl)-4,5-dihydro-1,2,4-oxadiazol-3-yl)benzyl)benzenesulfonamide (Compound 86)

### Step 1:—4-Fluoro-N-(4-(5-(trifluoromethyl)-1,2,4-oxadiazol-3-yl)benzyl)benzenesulfonamide

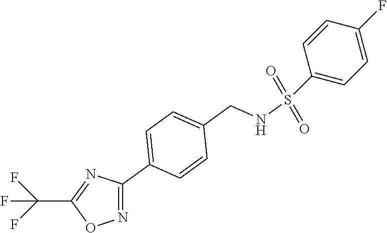 [SMILES: O=S(=O)(NCc1ccc(-c2noc(C(F)(F)F)n2)cc1)c1ccc(F)cc1]

To a stirred solution of (4-(5-(trifluoroethyl)-1,2,4-oxadiazol-3-yl)phenyl)methanamine (1 g, 4.1 mmol) in dichloromethane (10 mL), triethylamine (1.4 mL, 10.3 mmol) and p-fluorobenzenesulfonyl chloride (0.96 g 4.9 mmol) were added at 0-5° C. The reaction mixture was stirred at 25° C. for 2 h. After completion of reaction, the reaction mixture was diluted with dichloromethane (100 mL) and the dichloromethane layer was washed twice by aqueous sodium bicarbonate solution (20 mL). The dichloromethane layer was dried over anhydrous sodium sulphate and concentrated under reduced pressure. The crude product obtained was purified by column chromatography on silica gel using eluent 60% ethyl acetate in hexane to obtain 4-fluoro-N-(4-(5-(trifluoromethyl)-1,2,4-oxadiazol-3-yl)benzyl)benzenesulfonamide (0.9 g, 54% yield).

### Step 2:—4-Fluoro-N-(4-(5-(trifluoromethyl)-4,5-dihydro-1,2,4-oxadiazol-3-yl)benzyl)benzenesulfonamide

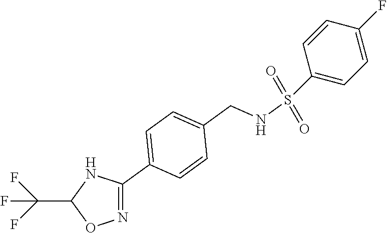 [SMILES: O=S(=O)(NCc1ccc(C2=NOC(C(F)(F)F)N2)cc1)c1ccc(F)cc1]

4-Fluoro-N-(4-(5-(trifluoromethyl)-4,5-dihydro-1,2,4-oxadiazol-3-yl)benzyl)benzenesulfonamide was prepared by the procedure analogous to that of the compound 21.

### Example 30:—Preparation of 4-fluoro-N-(4-(3-(trifluoromethyl)-1H-1,2,4-triazol-5-yl)benzyl)benzenesulfonamide (Compound 90)

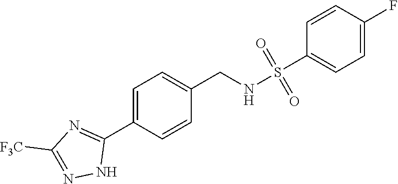 [SMILES: O=S(=O)(NCc1ccc(-c2nc(C(F)(F)F)n[nH]2)cc1)c1ccc(F)cc1]

4-Fluoro-N-(4-(3-(trifluoromethyl)-1H-1,2,4-triazol-5-yl)benzyl)benzenesulfonamide (0.27 g, 61% yield) was prepared by the procedure analogous to that of the compound 27 from 4-fluoro-N-(4-(5-(trifluoromethyl)-1,2,4-oxadiazol-3-yl)benzyl)benzenesulfonamide.

### Example 31:—Preparation of N-(4-(3-(trifluoromethyl)-1,2,4-oxadiazol-5-yl)benzyl)cyclobutanecarboxamide (Compound 87)

### Step 1-N-(4-(5-(trifluoromethyl)-1,2,4-oxadiazol-3-yl)benzyl)cyclobutanecarboxamide

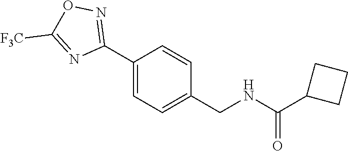 [SMILES: O=C(NCc1ccc(-c2noc(C(F)(F)F)n2)cc1)C1CCC1]

To a stirred solution of (4-(5-(trifluoromethyl)-1,2,4-oxadiazol-3-yl)phenyl)methanamine (0.5 g, 2.1 mmol) in dichloromethane (10 mL), triethylamine (0.43 ml, 3.1 mmol) and cyclobutanecarbonyl chloride (0.47 mL, 4.1 mmol) were added at 0-5° C. The reaction mixture was stirred at 25° C. for 2 h.

After completion of reaction, the reaction mixture was diluted with dichloromethane (100 mL) and the dichloromethane layer was washed twice by aqueous sodium bicarbonate solution (20 mL). The dichloromethane layer was dried over anhydrous sodium sulphate and concentrated under reduced pressure. The crude product was purified by column chromatography on silica gel using eluent 60% ethyl acetate in hexane to obtain N-(4-(5-(trifluoromethyl)-1,2,4-oxadiazol-3-yl)benzyl)cyclobutanecarboxamide (0.5 g, 75% yield).

### Step 2—N-(4-(3-(trifluoromethyl)-1,2,4-oxadiazol-5-yl)benzyl)cyclobutanecarboxamide

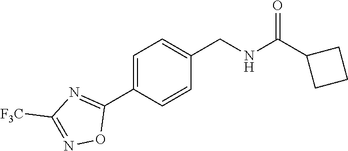 [SMILES: O=C(NCc1ccc(-c2nc(C(F)(F)F)no2)cc1)C1CCC1]

A solution of hydroxylamine hydrochloride (385 mg, 5.5 mmol) and potassium tert-butoxide (621 mg, 5.5 mmol) in dimethylformamide (8 mL) was stirred for 15 min at 25° C. To this stirred solution N-(4-(5-(trifluoromethyl)-1,2,4-oxadiazol-3-yl)benzyl)cyclobutanecarboxamide (450 mg, 1.4 mmol) was added at 25° C. Then the reaction mixture was stirred at 25° C. for 16 h. The reaction mixture was diluted with ethyl acetate (60 mL) and washed thrice with water (20 mL). The ethyl acetate layer was dried over anhydrous sodium sulphate and concentrated under reduced pressure. The crude product was purified by column chromatography on silica gel using eluent 50% ethyl acetate in hexane to obtain N-(4-(3-(trifluoromethyl)-1,2,4-oxadiazol-5-yl)benzyl)cyclobutanecarboxamide (202 mg, 45% yield).

### Example 32:—Preparation of 4-fluoro-N-(4-(3-(trifluoromethyl)-1,2,4-oxadiazol-5-yl)benzyl)benzenesulfonamide (Compound 92)

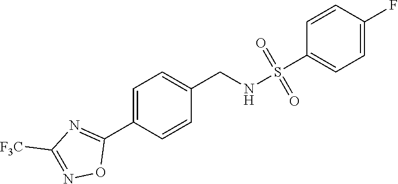 [SMILES: O=S(=O)(NCc1ccc(-c2nc(C(F)(F)F)no2)cc1)c1ccc(F)cc1]

4-Fluoro-N-(4-(3-(trifluoromethyl)-1,2,4-oxadiazol-5-yl)benzyl)benzenesulfonamide (0.137 g, 54% yield) was prepared by procedure analogous to that of the compound 87 from 4-fluoro-N-(4-(5-(trifluoromethyl)-1,2,4-oxadiazol-3-yl)benzyl)benzenesulfonamide.

### Example 33:—Preparation of methyl(phenyl)((4-(3-(trifluoromethyl)-1,2,4-oxadiazol-5-yl)benzyl)imino)-λ⁶-sulfanone (Compound 93)

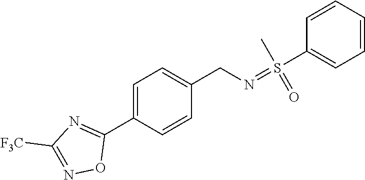 [SMILES: CS(=O)(=NCc1ccc(-c2nc(C(F)(F)F)no2)cc1)c1ccccc1]

Methyl(phenyl)((4-(3-(trifluoromethyl)-1,2,4-oxadiazol-5-yl)benzyl)imino)-λ⁶-sulfanone (0.23 g, 77% yield) was prepared by procedure analogous to that of the compound 87 from methyl(phenyl)((4-(5-(trifluoromethyl)-1,2,4-oxadiazol-3-yl)benzyl)imino)-λ⁶-sulfanone.

### Example 34:—Preparation of 1-isopropyl-3-(4-(3-(trifluoromethyl)-1,2,4-oxadiazol-5-yl)benzyl)urea (Compound 88)

### Step 1—1-Isopropyl-3-(4-(5-(trifluoromethyl)-1,2,4-oxadiazol-3-yl)benzyl)urea

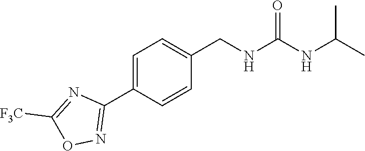 [SMILES: CC(C)NC(=O)NCc1ccc(-c2noc(C(F)(F)F)n2)cc1]

To a stirred solution of (4-(5-(trifluoromethyl)-1,2,4-oxadiazol-3-yl)phenyl)methanamine (1.3 g, 5.4 mmol) in tetrahydrofuran (10 mL), triethylamine (1.12 mL, 8 mmol) and 2-isocyanatopropane (0.64 mL, 6.4 mmol) were added at 0-5° C. The reaction mixture was stirred at 25° C. for 2 h. After completion of reaction, the reaction mixture was diluted with dichloromethane (100 mL) and dichloromethane layer was washed by aqueous sodium bicarbonate solution (40 mL) and water (30 mL). The reaction mixture was dried over anhydrous sodium sulphate and concentrated under reduced pressure. The crude product was purified by column chromatography on silica gel using eluent 40% ethyl acetate in hexane to obtain 1-isopropyl-3-(4-(5-(trifluoromethyl)-1,2,4-oxadiazol-3-yl)benzyl)urea (0.9 g, 51% yield).

### Step 2—1-Isopropyl-3-(4-(3-(trifluoromethyl)-1,2,4-oxadiazol-5-yl)benzyl)urea

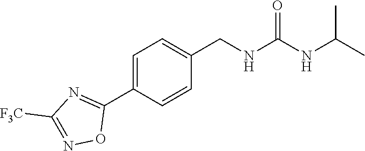 [SMILES: CC(C)NC(=O)NCc1ccc(-c2nc(C(F)(F)F)no2)cc1]

1-Isopropyl-3-(4-(3-(trifluoromethyl)-1,2,4-oxadiazol-5-yl)benzyl)urea (0.1 g, 39% yield) was prepared by procedure analogous to that of the compound 87 from 1-isopropyl-3-(4-(5-(trifluoromethyl)-1,2,4-oxadiazol-3-yl)benzyl)urea.

### Example 35:—Preparation of 1-isopropyl-3-(4-(3-(trifluoromethyl)-1H-1,2,4-triazol-5-yl)benzyl)urea (Compound 89)

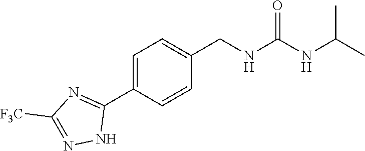 [SMILES: CC(C)NC(=O)NCc1ccc(-c2nc(C(F)(F)F)n[nH]2)cc1]

1-Isopropyl-3-(4-(3-(trifluoromethyl)-1H-1,2,4-triazol-5-yl)benzyl)urea (71 mg, 31% yield) was prepared by procedure analogous to that of the compound 27 from 1-isopropyl-3-(4-(5-(trifluoromethyl)-1,2,4-oxadiazol-3-yl)benzyl)urea.

### Example 36:—Preparation of 1-isopropyl-3-(4-(5-(trifluoromethyl)-4,5-dihydro-1,2,4-oxadiazol-3-yl)benzyl)urea (Compound 91)

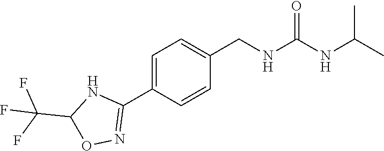 [SMILES: CC(C)NC(=O)NCc1ccc(C2=NOC(C(F)(F)F)N2)cc1]

1-Isopropyl-3-(4-(5-(trifluoromethyl)-4,5-dihydro-1,2,4-oxadiazol-3-yl)benzyl)urea (96 mg, 38% yield) was prepared by procedure analogous to that of the compound 21 from 1-isopropyl-3-(4-(5-(trifluoromethyl)-1,2,4-oxadiazol-3-yl)benzyl)urea.

### Example 37:—Preparation of (4-methoxyphenyl)(methyl)((4-(5-(trifluoromethyl)isoxazol-3-yl)phenyl)imino)-λ⁶-sulfanone (Compound 94)

### Step 1:—Ethyl-4-(4,4,4-trifluoro-3-oxobutanoyl)benzoate

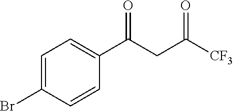 [SMILES: O=C(CC(=O)C(F)(F)F)c1ccc(Br)cc1]

To a stirred suspension of sodium methoxide (0.6 g, 11 mmol) in toluene (10 mL), ethyl-2,2,2-trifluoroacetate (0.90 mL, 7.54 mmol) was added at 0° C. and stirred for 30 min. Then 1-(4-bromophenyl)ethan-1-one (1 g, 5.02 mmol) was added portion wise. The resulting reaction mixture was allowed to stir at 25° C. for further 3 h. After completion of reaction, the reaction was then cooled to 0-5° C. and washed with aqueous 5% sulfuric acid solution (20 mL). Obtained solid mass was dissolved in ethyl acetate (30 mL), the ethyl acetate layer was washed with brine solution (10 mL), dried over anhydrous magnesium sulfate, filtered, and concentrated under reduced pressure to obtain 1-(4-bromophenyl)-4,4,4-trifluorobutane-1,3-dione (1.3 g, 88% yield).

### Step 2:—3-(4-Bromophenyl)-5-(trifluoromethyl)-4,5-dihydroisoxazol-5-01

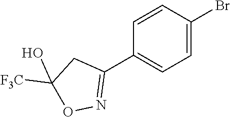 [SMILES: OC1(C(F)(F)F)CC(c2ccc(Br)cc2)=NO1]

A solution of 1-(4-bromophenyl)-4,4,4-trifluorobutane-1,3-dione (1.5 g, 5.1 mmol) in concentrated hydrochloric acid (10 mL) was treated with hydroxylamine hydrochloride (0.42 g, 6.1 mmol) in methanol (5 mL) and stirred at 60-70° C. for 16 h. After completion of reaction, the reaction mixture was cooled and the resulting solid was extracted with ethyl acetate (30 mL). The ethyl acetate layer was washed with a brine solution (10 mL), dried over anhydrous magnesium sulfate, filtered and concentrated under reduced pressure to obtain a crude product. The crude product was purified by column chromatography on silica gel using eluent 50% ethyl acetate in hexane to obtain 3-(4-bromophenyl)-5-(trifluoromethyl)-4,5-dihydroisoxazol-5-ol (1.02 g, 65% yield).

### Step 3:—3-(4-Bromophenyl)-5-(trifluoromethyl)isoxazole

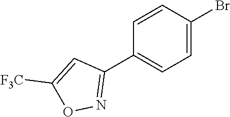 [SMILES: FC(F)(F)c1cc(-c2ccc(Br)cc2)no1]

A solution of 3-(4-bromophenyl)-5-(trifluoromethyl)-4,5-dihydroisoxazol-5-ol (6.0 g, 19.3 mmol) in trifluoroacetic acid (25 mL) was stirred at 80-90° C. for 24 h. After completion of reaction, the reaction mixture was cooled to 25° C. and extracted twice with ethyl acetate (50 mL). The combined ethyl acetate layers were washed twice with saturated aqueous sodium carbonate solution (30 mL), dried over anhydrous sodium sulfate and concentrated under reduced pressure to obtain a crude product. The crude product was purified by column chromatography on silica gel using eluent 30% ethyl acetate in hexane to obtain 3-(4-bromophenyl)-5-(trifluoromethyl)isoxazole (4.85 g, 86% yield).

### Step 4:—(4-Methoxyphenyl)(methyl)((4-(5-(trifluoromethyl)isoxazol-3-yl)phenyl)imino)-λ⁶-sulfanone

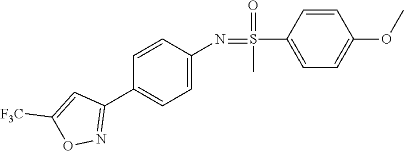 [SMILES: COc1ccc(S(C)(=O)=Nc2ccc(-c3cc(C(F)(F)F)on3)cc2)cc1]

To a stirred solution of 3-(4-bromophenyl)-5-(trifluoromethyl)isoxazole (0.35 g, 1.2 mmol) in dry toluene (10 mL), imino(4-methoxyphenyl)(methyl)-λ⁶-sulfanone (0.27 g, 1.44 mmol) and cesium carbonate (0.78 g, 2.4 mmol) were added under nitrogen atmosphere. The mixture was degassed with nitrogen for 10 min and then (±)-2,2′-bis(diphenylphosphino)-1,1′-binaphthalene (75 mg, 0.12 mmol) and palladium(II) acetate (13 mg, 0.060 mmol) were added. The resulting reaction mixture was degassed with nitrogen for 10 min and stirred at 110° C. for 12 h. After completion of the reaction, the reaction mixture was cooled to 25° C. and diluted with ethyl acetate (50 mL). The ethyl acetate layer was washed thrice with water (15 mL), dried over anhydrous sodium sulfate and concentrated under reduced pressure to obtain a crude product. The crude product was purified by column chromatography on silica gel using eluent 30% ethyl acetate in hexane to obtain (4-methoxyphenyl)(methyl)((4-(5-(trifluoromethyl)isoxazol-3-yl)phenyl)imino)-λ⁶-sulfanone (0.115 g, 24% yield).

TABLE 16
The following compounds were prepared by the procedure analogous to that of the
compound 94

| Compound | | | |
|---|---|---|---|
| no. | Structure | Iupac name | Yield |
| 95 | 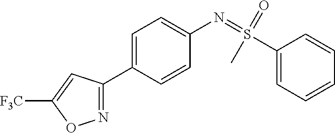 [SMILES: CS(=O)(=Nc1ccc(-c2cc(C(F)(F)F)on2)cc1)c1ccccc1] | methyl(phenyl)((4-(5- (trifluoromethyl)isoxazol-3- yl)phenyl)imino)-λ⁶-sulfanone | 0.102 g, 23% yield |
| 97 | 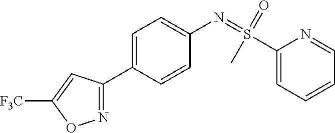 [SMILES: CS(=O)(=Nc1ccc(-c2cc(C(F)(F)F)on2)cc1)c1ccccn1] | methyl(pyridin-2-yl)((4-(5- (trifluoromethyl)isoxazol-3- yl)phenyl)imino)-λ⁶-sulfanone | 0.102 g, 16% yield |

### Example 38:—Preparation of tert-butyl (4-(5-(trifluoromethyl)isoxazol-3-yl)phenyl)carbamate (Compound 96)

### Step 1—tert-butyl (4-(5-(trifluoromethyl)isoxazol-3-yl)phenyl)carbamate

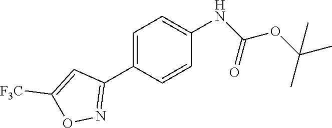 [SMILES: CC(C)(C)OC(=O)Nc1ccc(-c2cc(C(F)(F)F)on2)cc1]

To a stirred solution of 3-(4-bromophenyl)-5-(trifluoromethyl)isoxazole (3.0 g, 10.3 mmol) in dry toluene (20 mL), tert-butyl carbamate (1.45 g, 12.3 mmol) and cesium carbonate (6.7 g, 20.5 mmol) were added under nitrogen atmosphere. The mixture was degassed with nitrogen for 10 min and then (±)-2,2′-bis(diphenylphosphino)-1,1′-binaphthalene (0.64 g, 1 mmol) and palladium(II) acetate (0.12 g, 0.51 mmol) were added to it. The resulting reaction mixture was degassed with nitrogen for 10 min and stirred at 110° C. for 6 h. After completion of the reaction, the reaction mixture was diluted with ethyl acetate (50 mL) and washed twice with water (15 mL). The ethyl acetate layer was separated, and concentrated under reduced pressure to obtain a crude product. The crude product was purified by column chromatography on silica gel using eluent 60% ethyl acetate in hexane to obtain tert-butyl (4-(5-(trifluoromethyl)isoxazol-3-yl)phenyl)carbamate (1 g, 30% yield).

### Example 39: Preparation of (4-methoxyphenyl)(methyl)((4-(5-(trifluoromethyl)-1,3,4-oxadiazol-2-yl)phenyl)imino)-λ⁶-sulfanone (Compound 100)

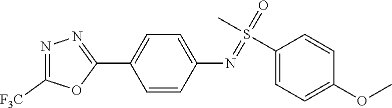 [SMILES: COc1ccc(S(C)(=O)=Nc2ccc(-c3nnc(C(F)(F)F)o3)cc2)cc1]

### Step-1:—5-(4-Bromophenyl)-1H-tetrazole

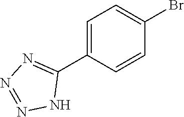

To a stirred solution of 4-bromobenzonitrile (10 g, 55 mmol) in N, N-dimethyl formamide (90 mL), sodium azide (3.9 g, 60 mmol) was added at 0° C. and then ceric ammonium nitrate (3 g, 5.5 mmol) was added portion wise. The resulting reaction mixture was stirred at 110° C. for 6 h. After completion of the reaction, the reaction mixture was cooled to 0° C., then 10% solution of acetic acid (9.4 ml, 165 mmol) was added to achieve pH 6. The precipitated solid was filtered through sintered funnel and washed by water (100 mL). The obtained solid was dried under reduced pressure to obtain 5-(4-bromophenyl)-1H-tetrazole (10 g, 81% yield).

### Step-2:—2-(4-bromophenyl)-5-(trifluoromethyl)-1,3,4-oxadiazole

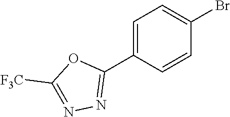 [SMILES: FC(F)(F)c1nnc(-c2ccc(Br)cc2)o1]

To a stirred solution of 5-(4-bromophenyl)-1H-tetrazole (1 g, 49 mmol) in pyridine (120 mL) trifluoroacetic anhydride (51.3 mL, 244 mmol) was added at 0° C. The resulting reaction mixture was stirred at 90° C. for 12 h. After completion of the reaction, the reaction mixture was extracted with ethyl acetate (140 mL) and washed twice with aqueous 1N hydrochloric acid solution (60 mL). The ethyl acetate layer was dried over anhydrous sodium sulphate and concentrated to obtain a crude product. The crude product was purified by column chromatography on silica gel using eluent 30% ethyl acetate in hexane to obtain 2-(4-bromophenyl)-5-(trifluoromethyl)-1,3,4-oxadiazole (4.2 g, 29% yield).

### Step-3:—(4-methoxyphenyl)(methyl)((4-(5-(trifluoromethyl)-1,3,4-oxadiazol-2-yl)phenyl)imino)-λ⁶-sulfanone

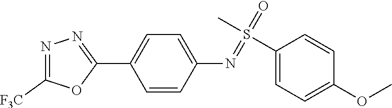 [SMILES: COc1ccc(S(C)(=O)=Nc2ccc(-c3nnc(C(F)(F)F)o3)cc2)cc1]

To a stirred solution of 2-(4-bromophenyl)-5-(trifluoromethyl)-1,3,4-oxadiazole (0.25 g, 0.85 mmol) in toluene (20 mL), cesium carbonate (0.42 g, 1.3 mmol) and imino(4-methoxyphenyl)(methyl)-λ⁶-sulfanone (0.17 g, 0.94 mmol) were added under nitrogen atmosphere at 25° C. The reaction mixture was degassed by nitrogen for 10 min and then (±)-2,2′-bis(diphenylphosphino)-1,1′-binaphthalene (0.08 g, 0.13 mmol) and palladium(II) acetate (0.015 g, 0.068 mmol) were added. The resulting reaction mixture was degassed by nitrogen for 10 min and stirred at 120° C. for 12 h. After completion of the reaction, the reaction mixture was diluted with ethyl acetate (25 mL) and washed with water (15 mL). The ethyl acetate layer was dried over anhydrous sodium sulphate and concentrated to obtain a crude product. The crude product was purified by column chromatography on silica gel using eluent 30% ethyl acetate in hexane to obtain (4-methoxyphenyl)(methyl)((4-(5-(trifluoromethyl)-1,3,4-oxadiazol-2-yl)phenyl)imino)-λ⁶-sulfanone (0.25 g, 74% yield).

TABLE 17
The following compounds were prepared by the procedure analogous to that of the
compound 100

| Compound | | | |
|---|---|---|---|
| no. | Structure | Iupac name | Yield |
| 98 | 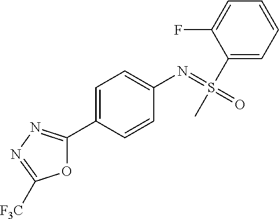 [SMILES: CS(=O)(=Nc1ccc(-c2nnc(C(F)(F)F)o2)cc1)c1ccccc1F] | (2-fluorophenyl)(methyl)((4-(5- (trifluoromethyl)-1,3,4-oxadiazol-2- yl)phenyl)imino)-λ⁶-sulfanone | 0.15 g, 38% yeld |
| 99 | 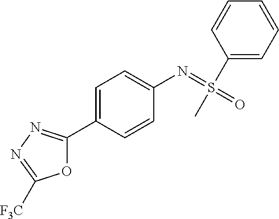 [SMILES: CS(=O)(=Nc1ccc(-c2nnc(C(F)(F)F)o2)cc1)c1ccccc1] | methyl(phenyl)((4-(5- (trifluoromethyl)-1,3,4-oxadiazol-2- yl)phenyl)imino)-λ⁶-sulfanone | 0.22 g, 58% yield |
| 101 | 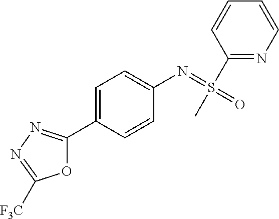 [SMILES: CS(=O)(=Nc1ccc(-c2nnc(C(F)(F)F)o2)cc1)c1ccccn1] | methyl(pyridin-2-yl)((4-(5- (trifluoromethyl)-1,3,4-oxadiazol-2- yl)phenyl)imino)-λ⁶-sulfanone | 87 mg, 23% yield |
| 102 | 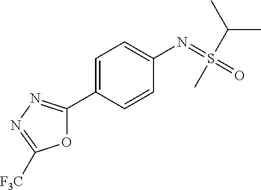 [SMILES: CC(C)S(C)(=O)=Nc1ccc(-c2nnc(C(F)(F)F)o2)cc1] | isopropyl(methyl)((4-(5- (trifluoromethyl)-1,3,4-oxadiazol-2- yl)phenyl)imino)-λ⁶-sulfanone | 0.12 g, 36% yield |

### Example 40:—Preparation of tert-butyl (4-(5-(trifluoromethyl)-1,3,4-oxadiazol-2-yl)phenyl)carbamate (Compound 103)

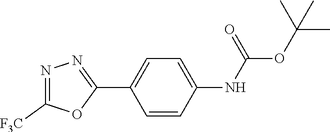 [SMILES: CC(C)(C)OC(=O)Nc1ccc(-c2nnc(C(F)(F)F)o2)cc1]

To a stirred solution of 2-(4-bromophenyl-5-(trifluoromethyl)-1,3,4-oxadiazole (1.5 g, 5.12 mmol) in toluene (20 mL), cesium carbonate (215 g, 7.7 mmol) and tert-butyl carbamate (0.72 g, 6.1 mmol) were added under nitrogen atmosphere at 25° C. The mixture was degassed by nitrogen for 10 min, then (±)-2,2′-bis(diphenylphosphino)-1,1′-binaphthalene (0.48 g, 0.77 mmol) and palladium(II) acetate (0.09 g, 0.41 mmol) were added. The resulting reaction mixture was degassed by nitrogen for 10 min and stirred at 110° C. for 12 h. After completion of the reaction, the reaction mixture was diluted with ethyl acetate (25 mL) and washed with water (15 mL). The ethyl acetate layer was dried over anhydrous sodium sulphate and concentrated under reduced pressure to obtain a crude product. The crude product was purified by column chromatography on silica gel using eluent 30% of ethyl acetate in hexane to obtain tert-butyl (4-(5-(trifluoromethyl)-1,3,4-oxadiazol-2-yl)phenyl)carbamate (0.55 g, 33% yield).

### Example 41:—Preparation of Preparation of N-(4-(5-(trifluoromethyl)-1,3,4-oxadiazol-2-yl)phenyl)cyclopropanecarboxamide (Compound 104)

### Step 1:—4-(5-(Trifluoromethyl)-1,3,4-oxadiazol-2-yl)aniline

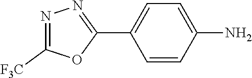 [SMILES: Nc1ccc(-c2nnc(C(F)(F)F)o2)cc1]

To a stirred solution of tert-butyl (4-(5-(trifluoromethyl)-1,3,4-oxadiazol-2-yl)phenyl)carbamate (0.5 g, 1.52 mmol) in dichloromethane (8 mL), trifluoroacetic acid (2 mL) was added under nitrogen atmosphere at 0° C. The reaction was stirred at 25° C. for 2 h. After completion of the reaction, the reaction mixture was concentrated under reduced pressure to obtain 4-(5-(trifluoromethyl)-1,3,4-oxadiazol-2-yl)aniline (0.3 g, 86% yield).

### Step 2:—N-(4-(5-(trifluoromethyl)-1,3,4-oxadiazol-2-yl)phenyl)cyclopropanecarboxamide

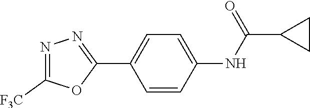 [SMILES: O=C(Nc1ccc(-c2nnc(C(F)(F)F)o2)cc1)C1CC1]

To a stirred solution of 4-(5-(trifluoromethyl)-1,3,4-oxadiazol-2-yl)aniline (0.11 g, 0.5 mmol) in dichloromethane (8 mL), N, N-diisopropylethylamine (0.42 mL, 2.4 mmol) was added and then stirred for 10 min. Cyclopropanecarbonyl chloride (0.05 mL, 0.6 mmol) was added and the reaction mixture was stirred 12 h at 25° C. The reaction mixture was diluted with dichloromethane (20 mL). The dichloromethane layer was washed by aqueous saturated sodium bicarbonate solution (10 mL), dried over anhydrous sodium sulphate and concentrated under reduced pressure to obtain a crude product. The crude product was purified by column chromatography on silica gel using eluent 30% of ethyl acetate in hexane to obtain N-(4-(5-(trifluoromethyl)-1,3,4-oxadiazol-2-yl)phenyl)cyclopropanecarboxamide (0.04 g, 29% yield).

### Example 42: Preparation of N-((4-methoxyphenyl)(methyl)(oxo)-λ⁶-sulfaneylidene)-4-(5-(trifluoromethyl)-1,3,4-oxadiazol-2-yl)benzamide (Compound 107)

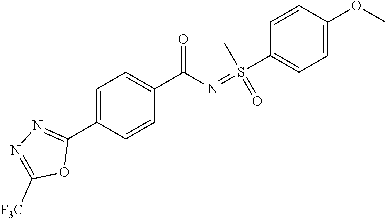 [SMILES: COc1ccc(S(C)(=O)=NC(=O)c2ccc(-c3nnc(C(F)(F)F)o3)cc2)cc1]

### Step-1:—methyl 4-(1H-tetrazol-5-yl)benzoate

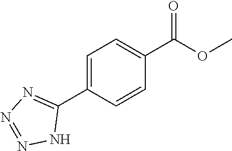

To a stirred solution of methyl 4-cyanobenzoate (6 g, 37 mmol) in N, N-dimethylformamide (40 mL), sodium azide (2.7 g, 41 mmol) was added at 0° C. and then ceric ammonium nitrate (2 g, 3.7 mmol) was added portion wise. The resulting reaction mixture was stirred at 110° C. for 6 h. After completion of the reaction, the reaction mixture was cooled to 0° C. and 10% solution of acetic acid (9 mL, 165 mmol) was added to achieve pH 6. The precipitated solid was filtered through sintered funnel and washed by water (100 mL). The obtained solid was dried under reduced pressure to obtain methyl 4-(1H-tetrazol-5-yl)benzoate (7 g, 92% yield).

### Step-2:—methyl 4-(5-(trifluoromethyl)-1,3,4-oxadiazol-2-yl)benzoate

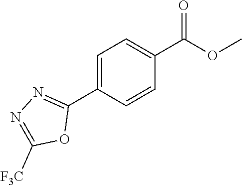 [SMILES: COC(=O)c1ccc(-c2nnc(C(F)(F)F)o2)cc1]

To a stirred solution of methyl 4-(1H-tetrazol-5-yl)benzoate (5 g, 24.5 mmol) in pyridine (60 mL) trifluoroacetic anhydride (17.29 mL, 122 mmol) was added at 0° C. The resulting reaction mixture was stirred at 90° C. for 12 h. After completion of the reaction, the reaction mixture was extracted with ethyl acetate (100 mL) and washed twice with 1N hydrochloric acid solution (50 mL). The ethyl acetate layer was dried over anhydrous sodium sulphate and concentrated under reduced pressure. The obtained crude product was purified by column chromatography on silica gel using eluent 40% of ethyl acetate in hexane to obtain methyl 4-(5-(trifluoromethyl)-1,3,4-oxadiazol-2-yl)benzoate (3.1 g, 46% yield).

### Step-3:—N-((4-methoxyphenyl)(methyl)(oxo)-λ⁶-sulfaneylidene)-4-(5-trifluoromethyl)-1,3,4-oxadiazol-2-yl)benzamide

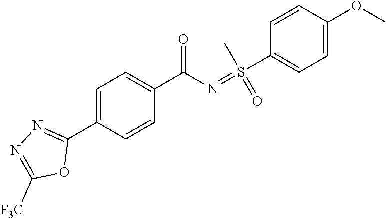 [SMILES: COc1ccc(S(C)(=O)=NC(=O)c2ccc(-c3nnc(C(F)(F)F)o3)cc2)cc1]

To a solution of methyl 4-(5-(trifluoromethyl)-1,3,4-oxadiazol-2-yl)benzoate (0.3 g, 1.1 mmol) in toluene (7 mL), imino(4-methoxyphenyl)(methyl)-λ⁶-sulfanone (0.51 g, 2.8 mmol) was added. Then trimethylaluminium (25% in hexane) (1.38 mL, 2.8 mmol) was added at 0-5° C. and stirred at 65° C. for 16 h. The reaction mixture was cooled to 25° C. and poured into a mixture of 5% aqueous acetic acid (7 mL) and ethyl acetate (15 mL) and stirred at 25° C. for 10 minutes. The ethyl acetate layer was isolated, washed by water (20 mL), dried over anhydrous sodium sulphate and concentrated under reduced pressure. The obtained crude product was purified by column chromatography on silica gel using eluent 40% of ethyl acetate in hexane to obtain N-((4-methoxyphenyl)(methyl)(oxo)-λ⁶-sulfanylidene)-4-(5-(trifluoromethyl)-1,3,4-oxadiazol-2-yl)benzamide (68 mg, 14% yield).

TABLE 18
The following compounds were prepared by the procedure analogous to that of the
compound 107

| Compound | | | |
|---|---|---|---|
| no. | Structure | Iupac name | Yield |
| 105 | 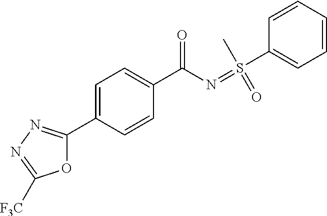 [SMILES: CS(=O)(=NC(=O)c1ccc(-c2nnc(C(F)(F)F)o2)cc1)c1ccccc1] | N-(methyl(oxo)(phenyl)-λ⁶- sulfaneylidene)-4-(5- (trifluoromethyl)-1,3,4-oxadiazol- 2-yl)benzamide | 0.09 g, 21% yield |
| 106 | 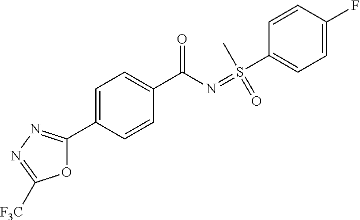 [SMILES: CS(=O)(=NC(=O)c1ccc(-c2nnc(C(F)(F)F)o2)cc1)c1ccc(F)cc1] | N-((4- fluorophenyl)(methyl)(oxo)-λ⁶- sulfaneylidene)-4-(5- (trifluoromethyl)-1,3,4-oxadiazol- 2-yl)benzamide | 0.16 g, 36% yield |

### Example 43:—Preparation of tert-butyl (4-(1-methyl-5-(trifluoromethyl)-1H-pyrazol-3-yl)phenyl)carbamate (Compound 108) and tert-butyl (4-(1-methyl-3-(trifluoromethyl)-1H-pyrazol-5-yl)phenyl)carbamate (Compound 109)

### Step 1:—3-(4-Bromophenyl)-5-(trifluoromethyl)-1H-pyrazole

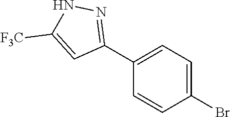 [SMILES: FC(F)(F)c1cc(-c2ccc(Br)cc2)n[nH]1]

To a stirred solution of 1-(4-bromophenyl)-4,4,4-trifluorobutane-1,3-dione (5.8 g, 19.7 mmol) in ethanol (2 mL), hydrazine hydrate (4.78 mL, 98 mmol) was added and refluxed at 90° C. for 18 h. After completion of the reaction, the reaction mixture was concentrated and quenched with crushed ice (100 g) with stirring. The precipitate was filtered and solid was dried under reduced pressure to obtain 3-(4-bromophenyl)-5-(trifluoromethyl)-1H-pyrazole (5.62 g, 98% yield).

### Step 2—3-(4-bromophenyl)-1-methyl-5-(trifluoromethyl)-1H-pyrazole and 5-(4-bromophenyl)-1-methyl-3-(trifluoromethyl)-1H-pyrazole

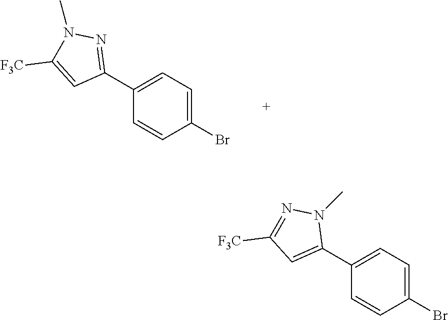 [SMILES: Cn1nc(-c2ccc(Br)cc2)cc1C(F)(F)F ; Cn1nc(C(F)(F)F)cc1-c1ccc(Br)cc1]

To a solution of 3-(4-bromophenyl)-5-(trifluoromethyl)-1H-pyrazole (5.62 g, 19.31 mmol) and acetonitrile (5 mL), potassium carbonate (6.67 g, 48.3 mmol) and methyl iodide (1.45 mL, 23.2 mmol) were added at 0-5° C. and stirred at 25° C. for 2 h. The resulting reaction mixture was filtered through celite and washed by ethyl acetate (30 mL). The combined filtrate was concentered under reduced pressure to obtain a crude product (4.7 g) as a mixture of 3-(4-bromophenyl)-1-methyl-5-(trifluoromethyl)-1H-pyrazole and 5-(4-bromophenyl)-1-methyl-3-(trifluoromethyl)-1H-pyrazole.

### Step 3:—tert-butyl (4-(1-methyl-5-(trifluoromethyl)-1H-pyrazol-3-yl)phenyl)carbamate and tert-butyl(4-(1-methyl-3-(trifluoromethyl)-1H-pyrazol-5-yl)phenyl)carbamate

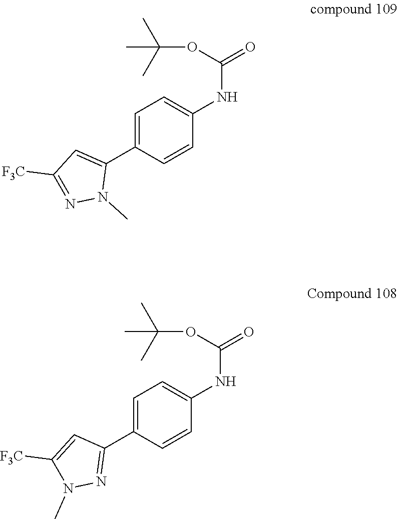 [SMILES: Cn1nc(C(F)(F)F)cc1-c1ccc(NC(=O)OC(C)(C)C)cc1 ; Cn1nc(-c2ccc(NC(=O)OC(C)(C)C)cc2)cc1C(F)(F)F]

To a stirred solution of 3-(4-bromophenyl)-1-methyl-5-(trifluoromethyl)-1H-pyrazole and 5-(4-bromophenyl)-1-methyl-3-(trifluoromethyl)-1H-pyrazole (0.3 g, 1 mmol) and toluene (8 mL) in seal tube, tert-butyl carbamate (0.23 g, 2 mmol) and cesium carbonate (0.8 g, 2.5 mmol) were added under nitrogen atmosphere. The mixture was degassed with nitrogen for 10 min and (±)-2,2′-bis(diphenylphosphino)-1,1′-binaphthalene (0.12 g, 0.2 mmol) was added. The mixture was degassed again with nitrogen for 5 min and palladium(II) acetate (22 mg, 0.1 mmol) was added. The resulting reaction mixture was degassed with nitrogen for 10 min and stirred at 100° C. for 18 h. After completion of the reaction, the reaction mixture was diluted with water and extracted with ethyl acetate (20 mL). The ethyl acetate layer was separated, dried over anhydrous sodium sulphate and concentrated under reduced pressure to obtain a crude product. The crude product was purified by column chromatography on silica gel using eluent 50% ethyl acetate in hexane to obtain tert-butyl (4-(1-methyl-5-(trifluoromethyl)-1H-pyrazol-3-yl)phenyl)carbamate (87 mg, 0.26 mmol, 26% yield) and tert-butyl (4-(1-methyl-3-(trifluoromethyl)-1H-pyrazol-5-yl)phenyl)carbamate (0.11 g, 0.32 mmol, 32% yield).

### Example 44:—Preparation of N-(4-(1-methyl-3-(trifluoromethyl)-1H-pyrazol-5-yl)phenyl)cyclopropanecarboxamide (Compound 110)

### Step 1:—4-(1-Methyl-3-(trifluoromethyl)-1H-pyrazol-5-yl)benzenaminium 2,2,2-trifluoroacetate

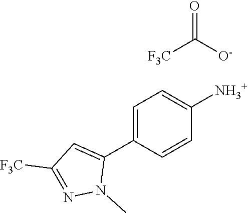 [SMILES: O=C([O-])C(F)(F)F ; Cn1nc(C(F)(F)F)cc1-c1ccc([NH3+])cc1]

To a stirred solution of tert-butyl (4-(1-methyl-3-(trifluoromethyl)-1H-pyrazol-5-yl)phenyl)carbamate (500 mg, 1.4 mmol) in dichloromethane (8 mL), trifluoroacetic acid (2 mL) was added under nitrogen atmosphere at 0° C. The reaction was allowed to stir at 25° C. for 2 h. After completion of the reaction, the reaction mixture was concentrated under reduced pressure to obtain 4-(1-methyl-3-(trifluoromethyl)-1H-pyrazol-5-yl)benzenaminium 2,2,2-trifluoroacetate (520 mg, 100% yield).

### Step 2:—N-(4-(1-methyl-3-(trifluoromethyl)-1H-pyrazol-5-yl)phenyl)cyclopropanecarboxamide

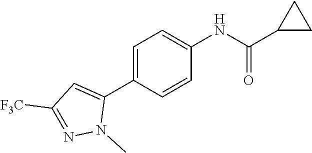 [SMILES: Cn1nc(C(F)(F)F)cc1-c1ccc(NC(=O)C2CC2)cc1]

To a stirred solution of 4-(1-methyl-3-(trifluoromethyl)-1H-pyrazol-5-yl)benzenaminium 2,2,2-trifluoroacetate (300 mg, 1.18 mmol) in dichloromethane (10 mL), N, N-diisopropyl ethyl amine (1.1 mL, 6.22 mmol) was added and stirred for 10 min. Cyclopropanecarbonyl chloride (0.135 mL, 1.5 mmol) was added at 0-5° C. slowly and stirred for 16 h at 25° C. After completion of reaction, the reaction mixture was extracted with dichloromethane (10 mL) and washed by sodium bicarbonate. The dichloromethane layer was dried over anhydrous sodium sulphate and concentrated under reduced pressure to obtain N-(4-(1-methyl-3-(trifluoromethyl)-1H-pyrazol-5-yl)phenyl)cyclopropanecarboxamide (144 mg, 37% yield).

### Example 45:—Preparation of 1-isopropyl-3-(4-(1-methyl-5-(trifluoromethyl)-1H-pyrazol-3-yl)phenyl)urea (Compound 111)

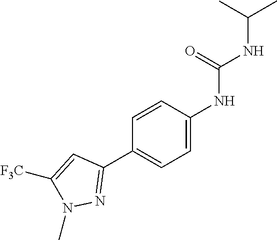 [SMILES: CC(C)NC(=O)Nc1ccc(-c2cc(C(F)(F)F)n(C)n2)cc1]

### Step 1—1-Isopropyl-3-(4-(1-methyl-5-(trifluoromethyl)-1H-pyrazol-3-yl)phenyl)urea

To a stirred solution of 4-(1-methyl-3-(trifluoromethyl)-1H-pyrazol-5-yl)benzenaminium 2,2,2-trifluoroacetate (300 mg, 1.2 mmol) in tetrahydrofuran (10 mL), triethylamine (0.4 mL, 3.1 mmol) was added slowly followed by slow addition of 2-isocyanatopropane (0.18 mL, 1.8 mmol) at 0-5° C. The resulting reaction mixture was stirred at 25° C. for 16 h. After completion of reaction, the reaction mixture was diluted with dichloromethane (30 mL) and washed twice with water (40 mL), dried over anhydrous sodium sulphate and concentrate under reduced pressure to obtain a crude product. The crude product was purified by flash column chromatography on silica gel using eluent 60% ethyl acetate in hexane to obtain 1-isopropyl-3-(4-(1-methyl-5-(trifluoromethyl)-1H-pyrazol-3-yl)phenyl)urea (75 mg, 19% yield).

### Example 46:—Preparation of N-ethyl-N-methyl-4-(3-(trifluoromethyl)-1,2,4-oxadiazol-5-yl)benzenesulfonamide (Compound 112)

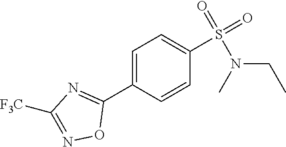 [SMILES: CCN(C)S(=O)(=O)c1ccc(-c2nc(C(F)(F)F)no2)cc1]

### Step 1: N-ethyl-4-isocyano-N-methylbenzenesulfonamide

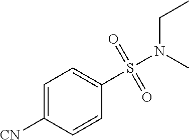

To a solution of 4-iso cyanobenzenesulfonyl chloride (150 mg, 0.74 mmol) in dichloromethane (10 mL), N-methylethanamine (57 mg, 1 mmol), triethylamine (0.26 mL, 1.9 mmol) were added at 0-5° C. and stirred at 25° C. for 1 h. The reaction mixture was diluted with water and extracted with ethyl acetate (20 mL). The ethyl acetate layer was washed with brine (10 mL), dried over anhydrous sodium sulphate and concentrated to obtain N-ethyl-4-isocyano-N-methylbenzenesulfonamide (164 mg, 98% yield).

### Step 2—-4-(N-ethyl-N-methylsulfamoyl)-N′-hydroxybenzimidamide

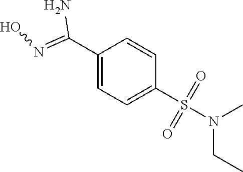 [SMILES: CCN(C)S(=O)(=O)c1ccc(C(N)=NO)cc1]

To a solution of methyl N-ethyl-4-isocyano-N-methylbenzenesulfonamide (160 mg, 0.7 mmol) in ethanol (15 mL), hydroxylamine solution (50% in water) (0.178 mL, 2.9 mmol) was added and stirred at 65° C. for 18 h. The reaction mixture was filtered, filtrate was concentrated and washed by ethyl acetate to obtain the 4-(N-ethyl-N-methylsulfamoyl)-N′-hydroxybenzimidamide (180 mg, 98% yield).

### Step 3—N-ethyl-N-methyl-4-(5-(trifluoromethyl)-1,2,4-oxadiazol-3-yl)benzenesulfonamide

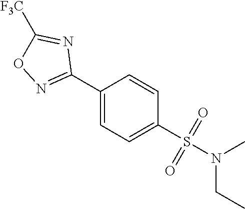 [SMILES: CCN(C)S(=O)(=O)c1ccc(-c2noc(C(F)(F)F)n2)cc1]

To a solution of 4-(N-ethyl-N-methylsulfamoyl)-N′-hydroxybenzimidamide (1.91 g, 7.42 mmol) and tetrahydrofuran (20 mL), trifluoroacetic anhydride (1.887 mL, 13.36 mmol) was added at 0-5° C. and stirred at 25° C. for 16 h. After completion of reaction, the reaction mixture was poured into ice cold mixture of ethyl acetate (80 mL) and saturated sodium bicarbonate (60 mL) with stirring (caution-pH must remain basic). The ethyl acetate layer was isolated and washed twice by saturated sodium bicarbonate solution (50 mL), dried over anhydrous sodium sulphate and concentrated under reduced pressure. The crude product was purified by column chromatography on silica gel using eluent 60% ethyl acetate in hexane to obtain N-ethyl-N-methyl-4-(5-(trifluoromethyl)-1,2,4-oxadiazol-3-yl)benzenesulfonamide (1.25 g, 50% yield).

### Step 4—N-ethyl-N-methyl-4-(3-(trifluoromethyl)-1,2,4-oxadiazol-5-yl)benzenesulfonamide

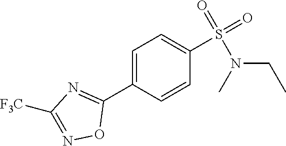 [SMILES: CCN(C)S(=O)(=O)c1ccc(-c2nc(C(F)(F)F)no2)cc1]

N-ethyl-N-methyl-4-(3-(trifluoromethyl)-1,2,4-oxadiazol-5-yl)benzenesulfonamide (90 mg, 76% yield) was prepared by procedure analogous to that of the compound 87 from N-ethyl-N-methyl-4-(5-(trifluoromethyl)-1,2,4-oxadiazol-3-yl)benzenesulfonamide.

### Example 47:—Preparation of N-ethyl-N-methyl-4-(3-(trifluoromethyl)-1H-1,2,4-triazol-5-yl)benzenesulfonamide (Compound 113)

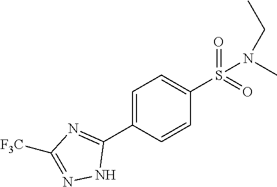 [SMILES: CCN(C)S(=O)(=O)c1ccc(-c2nc(C(F)(F)F)n[nH]2)cc1]

N-ethyl-N-methyl-4-(3-(trifluoromethyl)-1H-1,2,4-triazol-5-yl)benzenesulfonamide was prepared by procedure analogous to that of the compound 27 from N-ethyl-N-methyl-4-(5-(trifluoromethyl)-1,2,4-oxadiazol-3-yl)benzenesulfonamide (0.27 g, 89% yield).

### BIOLOGY EXAMPLES

### Example 1: Pyricularia oryzae (Rice Blast)

Compounds were dissolved in 0.3% DMSO & then added to Potato Dextrose Agar medium just prior to dispensing it into Petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds 1 16 17 24 27 30 42 67 82 and 84 at 300 ppm gave more than 70% control in these tests when compared to the untreated check which showed extensive disease development.

### Example 2: Rhizoctonia solani (Rice Sheath Blight/Potato Black Scurf)

Compounds were dissolved in 0.3% DMSO & then added to Potato Dextrose Agar medium just prior to dispensing it into Petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds 56 and 62 at 300 ppm gave more than 70% control in these tests when compared to the untreated check which showed extensive disease development.

### Example 3: Botrytis cinerea (Gray Mold)

Compounds were dissolved in 0.3% DMSO & then added to Potato Dextrose Agar medium just prior to dispensing it into Petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 22° C. temperature and 90% relative humidity for seven days and radial growth was measured. Compounds 40 and 42 at 300 ppm gave more than 70% in these tests when compared to the untreated check which showed extensive disease development.

### Example 4: Alternaria solani (Early Blight of Tomato/Potato)

Compounds were dissolved in 0.3% DMSO & then added to Potato Dextrose Agar medium just prior to dispensing it into Petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds 11 16 24 25 26 30 31 37 38 56 62 67 78 81 82 and 84 at 300 ppm gave more than 70% control in these tests when compared to the untreated check which showed extensive disease development.

### Example 5: Colletotrichum capsici (Anthracnose)

Compounds were dissolved in 0.3% DMSO & then added to Potato Dextrose Agar medium just prior to dispensing it into Petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured. Compounds 62 and 78 at 300 ppm gave more than 70% control in these tests when compared to the untreated check which showed extensive disease development.

### Example 6: Corynespora cassiicola (Leaf Spot of Tomato)

Compounds were dissolved in 0.3% DMSO & then added to Potato Dextrose Agar medium just prior to dispensing it into Petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 70% relative humidity for seven days and radial growth was measured. Compound 59 at 300 ppm gave more than 70% control in these tests when compared to the untreated check which showed extensive disease development.

### Example 7: Fusarium culmorum (Foot Rot of Cereals)

Compounds were dissolved in 0.3% DMSO & then added to Potato Dextrose Agar medium just prior to dispensing it into petri dishes. 5 ml medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 25° C. temperature and 60% relative humidity for seven days and radial growth was measured.

### Example 8: Phakopsora pachyrhizi Test in Soybean

Compounds were dissolved in 2% DMSO/Acetone & then mixed with water to the calibrated spray volume of 50 ml and poured into the spray bottles for further applications.

To test the preventive activity of compounds, healthy young soybean plants raised in the greenhouse were sprayed with active compound preparation at the stated application rates inside the spray cabinets using hallowcone nozzles. One day after treatment, the plants were inoculated with spore suspension containing 2.1×10⁶ Phakopsora pachyrhizi inoculum. The inoculated plants were then kept in greenhouse chamber at 250 C temperature & 90% Relative Humidity for disease expression.

A visual assessment of compound's performance was carried out by rating the disease severity (0-100% scale) on treated plants on 3, 7, 10 & 15 days after application. Efficacy (% control) of the compounds was calculated by comparing the disease rating in the treatment with the one of the untreated control. The sprayed plants were also assessed for compound's plant compatibility by recording symptoms like necrosis, chlorosis & stunting.

Compounds 1 3 20 21 22 23 24 25 29 30 34 40 43 44 47 56 57 65 66 67 69 70 71 72 74 77 80 82 83 showed >90% at 500 ppm control in these tests when compared to the untreated check which showed extensive disease development.

1. A compound of the Formula I, 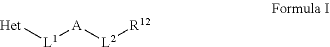
wherein;
Het is selected from the group consisting of Het-1 to Het-18; 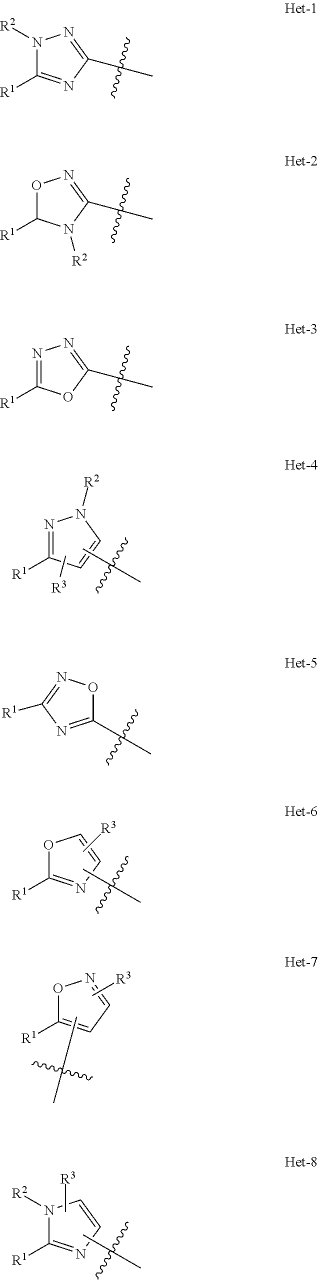 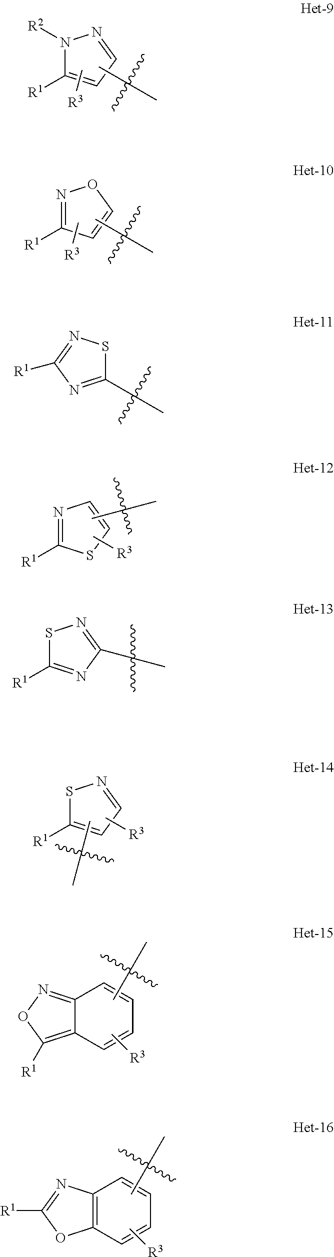 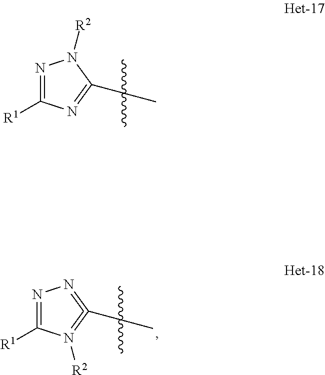
wherein, the expression “ [symbol] ” indicates the point of attachment to L¹;
R¹ is C₁-C₆ haloalkyl;
R² is selected from the group consisting of hydrogen, C₁-C₆-alkyl, C₁-C₆-haloalkyl, C₃-C₆-cycloalkyl, C₃-C₆-halocycloalkyl, C₁-C₆-alkoxy, C₁-C₆-haloalkoxy, C₃-C₆-cycloalkylalkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl- and C₃-C₆-halocycloalkylalkyl;
R³ is selected from the group consisting of hydrogen, halogen, C₁-C₆-alkyl, C₁-C₆-haloalkyl, C₃-C₆-cycloalkyl, C₁-C₆-alkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl and C₁-C₆-haloalkoxy;
L¹ is a direct bond, —CR⁴R⁵—, —C(═O)—, —CH₂C(═O)—, —O—, —S(═O)₀₋₂ or —NR⁶—, wherein, an expression “-” at the start and the end of the group indicates the point of attachment to either Het or A;
wherein, R⁴ and R⁵ are independently selected from the group consisting of hydrogen, halogen, cyano, C₁-C₄-alkyl, C₁-C₄-haloalkyl, C₃-C₆-cycloalkyl, C₃-C₆-halocycloalkyl, C₁-C₄-alkoxy and C₁-C₄-haloalkoxy; or
R⁴ and R⁵ together with the atoms to which they are attached may form 3- to 6-membered non aromatic carbocylic ring or heterocyclic ring which may be optionally substituted with halogen, C₁-C₂-alkyl, C₁-C₂-haloalkyl and C₁-C₂-alkoxy; and
R⁶ is selected from the group consisting of hydrogen, C₁-C₆-alkyl, C₁-C₆-haloalkyl, C₃-C₆-cycloalkyl, C₃-C₆-halocycloalkyl, C₁-C₆-alkoxy, C₁-C₆-haloalkoxy, C₃-C₆-cycloalkylalkyl and C₃-C₆-halocycloalkylalkyl;
A is phenyl or a 5- or 6-membered heteroaryl; wherein the heteroatoms of the heteroaryl are selected from N, O and S; and wherein the phenyl or the 5- or 6-membered heteroaryl may be unsubstituted or substituted with one or more identical or different R^{A} groups,
wherein, R^{A} is selected from the group consisting of hydrogen, halogen, cyano, nitro, sulfanyl, amino, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₂-C₆-alkenyloxy, C₂-C₆-haloalkenyloxy, C₂-C₆-alknyloxy, C₂-C₆-haloalknyloxy, C₁-C₆-haloalkoxy, C₃-C₈-cycloalkoxy, C₁-C₆-haloalkoxycarbonyl, C₁-C₆-alkylthio, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, C₁-C₆-dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, C₁-C₆-dialkylaminocarbonyl, C₁-C₆-alkoxycarbonyloxy, C₁-C₆-alkylaminocarbonyloxy and C₁-C₆-dialkylaminocarbonyloxy;
and wherein R^{A} may be optionally substituted with one or more identical or different R^{a} selected from the group consisting of halogen, cyano, nitro, sulfanyl, amino, hydroxy, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₁-C₆-alkoxy-C₁-C₄-alkyl, C₃-C₈-halocycloalkyl, C₁-C₆-alkylamino, di-C₁-C₆-alkylamino and C₃-C₈-cycloalkylamino;
L² is a direct bond or is selected from the group of —CR⁷R⁸—; —C(═O)—; —C(═S)—; —O—; —S(═O)₀₋₂—; —NR¹⁰—; 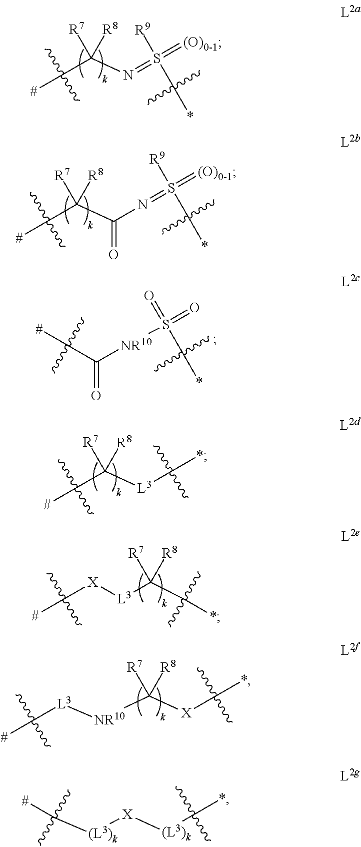
wherein X is a direct bond or —NR¹⁰—, or —O—, or —S(O)₀₋₂— or —C(═NR¹¹)—; or a 5-membered heteroaryl substituted or unsubstituted with one or more identical or different R^{L} is independently selected from the group consisting of halogen, amino, C₁-C₆-alkylamino, di-C₁-C₆-alkylamino, C₁-C₆-alkyl, C₁-C₆-alkoxy, C₂-C₆-alkenyl, C₂-C₆-alkynyl, C₃-C₈-cycloalkyl, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl and C₃-C₈-cycloalkoxy; and wherein R^{L} may be optionally substituted with one or more identical or different Rⁱ; wherein, Rⁱ is selected from the group consisting of halogen, cyano, C₁-C₆-alkyl, C₁-C₆-haloalkyl, C₁-C₆-alkoxy, C₁-C₆-haloalkoxy and C₃-C₈-cycloalkyl;
wherein,
k is an integer ranging from 0 to 4; expressions “-”, “#” and “*” indicate point of attachments to either A or R¹²;
L³ is a direct bond, —CR⁷R⁸—, —CH₂C(O)—, —C(═O)—, —C(═S)—, —O—, —S(═O)₀₋₂—, —S(O)₀₋₁(═N—R¹⁰)—, —S(═N—CN)—, —S(═N—NO₂)—, —S(═N—COR⁷)—, —S(═N—COOR¹¹)—, —S(═N—(S(═O)₂R⁹))—, —NR¹⁰—, —NR¹⁰(C(═O))O—, —CR⁷(═N)O;
wherein, R⁷ and R⁸ are independently selected from the group consisting of hydrogen, halogen, cyano, C₁-C₆-haloalkyl, C₁-C₆-alkoxy, C₂-C₆-alkenyl, C₂-C₆-alkynyl, C₃-C₈-cycloalkyl, C₃-C₈-cycloalkenyl, phenyl-C₁-C₆-alkyl, heteroaryl-C₁-C₆-alkyl, phenyl, naphthyl and a 3- to 10-membered saturated, partially unsaturated or aromatic mono- or bicyclic carbocyclic ring or heterocyclic ring, wherein the ring members of the heteroaryl of the heteroaryl-C₁-C₆-alkyl and the heterocyclic ring include C, N, O and S(O)₀₋₂ and the C ring members of the carbocylic ring or the heterocyclic ring may be replaced by one or more C(═O) and C(═S); and wherein R⁷ and R⁸ are independently unsubstituted or substituted with one or more identical or different R^{7a} selected from the group consisting of halogen, cyano, nitro, hydroxyl, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, amino-C₁-C₆-alkyl, di-C₁-C₆-alkylamino, NHSO₂—C₁-C₆-alkyl, —C(═O)—C₁-C₆-alkyl, C(═O)—C₁-C₆-alkoxy, C₁-C₆-alkylsulfonyl, hydroxy-C₁-C₆-alkyl, —C(═O)—NH₂, C(═O)—NH(C₁-C₆-alkyl), C₁-C₆-alkylthio-C₁-C₆-alkyl, C₁-C₆-alkylamino-C₁-C₆-alkyl, di-C₁-C₆-alkylamino-C₁-C₆-alkyl, aminocarbonyl-C₁-C₆-alkyl and C₁-C₆-alkoxy-C₁-C₆-alkyl; or
R⁷ and R⁸ together with the carbon atom to which they are bound form C(═O) or a vinyl group or a saturated, monocyclic 3- to 7-membered heterocycle or carbocycle, wherein the ring members of heterocyclic include C, N, O and S(O)₀₋₂; and wherein the vinyl group, the heterocyclic ring or the carbocyclic ring is unsubstituted or substituted with one or more identical or different R^{7b}, wherein R^{7b} is selected from the group consisting of halogen, cyano, nitro, hydroxyl, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, SO₂—C₁-C₆-alkyl, NHSO₂—C₁-C₆-alkyl, —C(═O)—C₁-C₆-alkyl, C(═O)—C₁-C₆-alkoxy, C₁-C₆-alkylsulfonyl, SO₂—CH₄CH₃ and SO₂-aryl;
R⁹ is selected from the group consisting of hydrogen; NR^{g}R^{h}, wherein, R^{g} and R^{h} are independently represent hydrogen, hydroxyl, cyano, C₁-C₄-alkyl, C₁-C₄-haloalkyl, C₁-C₄-alkoxy or C₃-C₈-cycloalkyl; (C═O)—Rⁱ, wherein, Rⁱ represents hydrogen, halogen, cyano, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, C₃-C₆-halocycloalkyl, C₁-C₄-alkoxy, and C₁-C₄-haloalkoxy; C₁₋₃-alkyl-S(O)₀₋₂R^{j}, wherein R^{j} represents hydrogen, halogen, cyano, C₁-C₆-alkyl, C₁-C₆-haloalkyl, C₁-C₆-alkoxy, C₁-C₆-haloalkoxy, C₃-C₈-cycloalkyl; C₁-C₆-alkyl-(C═O)—Rⁱ, CRⁱ═NR^{g}, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₁-C₆-haloalkoxy, C₃-C₈-cycloalkyl, C₄-C₈-cycloalkenyl, C₇-C₁₉-aralkyl, bicyclic C₅-C₁₂-alkyl, C₇-C₁₂-alkenyl, fused or non-fused or bicyclic C₃-C₁₃-carbocyclic ring or ring system; wherein one or more C atoms of the carbocyclic ring or ring system may be replaced by N, O, S(═O)₀₋₂, S(═O)₀₋₁(═NR¹⁰), C(═O), C(═S), C(═CR⁷R⁸) and C═NR¹⁰;
wherein, R⁹ may optionally be substituted with one or more identical or different substituents selected from the group consisting of hydrogen, halogen, cyano, nitro, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, C₁-C₆-haloalkoxycarbonyl, C₁-C₆-alkylthio, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, di-C₁-C₆-alkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, di-C₁-C₆-alkylaminocarbonyl, C₁-C₆-alkoxycarbonyloxy, C₁-C₆-alkylaminocarbonyloxy, di C₁-C₆-alkylaminocarbonyloxy, 5- to 11-membered spirocyclic ring and 3- to 6-membered carbocyclic or heterocyclic ring;
R¹⁰ is selected from the group consisting of hydrogen, C₁-C₆-alkyl, C₁-C₆-haloalkyl, C₁-C₆-alkyl-CN, C₁-C₆-alkoxy, C₂-C₆-alkenyl, C₂-C₆-alkynyl, C₃-C₈-cycloalkyl, C₃-C₈-cycloalkenyl, phenyl-C₁-C₆-alkyl, (C═O)—C₁-C₆-alkyl, C(═O)—C₁-C₆-alkoxy, 5- or 6-membered heteroaryl-C₁-C₆-alkyl, phenyl, naphthyl and a 3- to 10-membered saturated, partially unsaturated or aromatic mono- or bicyclic carbocyclic ring or heterocyclic ring, wherein the ring members of the heteroaryl of heteroaryl-C₁-C₆-alkyl and the mono- or bicyclic heterocycle are selected from C, N, O and S and wherein one or more C ring members of the carbocylic ring or the heterocyclic ring may be replaced by one or more groups selected from C(═O) and C(═S); and wherein R¹⁰ is unsubstituted or substituted with one or more identical or different R^{10a}; wherein, R^{10a} is selected from the group consisting of halogen, cyano, oxo, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, —C(═O)—C₁-C₆-alkyl, C(═O)—C₁-C₆-alkoxy, C₁-C₆-alkylsulfonyl, hydroxy-C₁-C₆-alkyl, —C(═O)—NH₂, C(═O)—NH(C₁-C₆-alkyl), C₁-C₆-alkylthio-C₁-C₆-alkyl, amino-C₁-C₆-alkyl, C₁-C₆-alkylamino-C₁-C₆-alkyl, di-C₁-C₆-alkylamino-C₁-C₆-alkyl, aminocarbonyl-C₁-C₆-alkyl and C₁-C₆-alkoxy-C₁-C₆-alkyl;
R¹¹ is selected from the group consisting of hydrogen, C₁-C₆-alkyl, C₁-C₆-haloalkyl, C₂-C₆-alkenyl, C₂-C₆-alkynyl, C₃-C₈-cycloalkyl, C₁-C₆-alkylthio, C₃-C₈-cycloalkenyl, phenyl-C₁-C₆-alkyl, heteroaryl-C₁-C₆-alkyl, phenyl, naphthyl and a 3- to 10-membered saturated, partially unsaturated or aromatic mono- or bicyclic carbocyclic or heterocyclic ring, wherein the ring members of the heteroaryl in the heteroaryl-C₁-C₆-alkyl and the heterocyclic ring include C, N, O and S(O)₀₋₂ and the C ring members of the carbocyclic ring or the heterocyclic ring may be replaced by one or more C(═O) and C(═S); and wherein R¹¹ is unsubstituted or substituted with one or more identical or different R^{11a} selected from the group consisting of halogen, cyano, nitro, hydroxyl, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, amino-C₁-C₆-alkyl and di-C₁-C₆-alkylamino; or
A, L² and R¹² together form a fragment A¹ 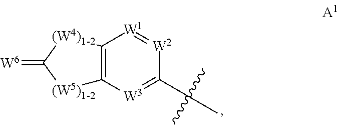
wherein W¹, W², W³, W⁴, and W⁵, independently are C or N, provided all are not N simultaneously; W⁶ is O or S; the expression “ [symbol] ” indicates the point of attachment to Het; and the fragment A¹ is substituted or unsubstituted with one or more identical or different R^{A};
R¹² is NR^{12a}R^{12b}, OR¹³, NR¹⁴NR^{12a}R^{12b}, R¹⁵, S(O)₀₋₂R¹⁶, COOR¹³, CONR^{12a}R^{12b}, COR¹⁵, NR^{12a}OR¹³;
wherein, R^{12a}, R^{12b}, and R¹⁴ are independently selected from the group consisting of hydrogen, C₁-C₆-alkyl, C₁-C₆-haloalkyl, C₁-C₆-alkoxy, C₂-C₆-alkenyl, C₂-C₆-alkynyl, C₃-C₈-cycloalkyl, C₃-C₈-cycloalkenyl, phenyl-C₁-C₆-alkyl, (C═O)—C₁-C₆-alkyl, C(═O)—C₁-C₆-alkoxy, C₁-C₆-alkoxyimino-C₁-C₆-alkyl, C₂-C₆-alkenyloxyimino-C₁-C₆-alkyl, C₂-C₆-alkynyloxyimino-C₁-C₆-alkyl, aminocarbonyl-C₁-C₆-alkyl, C₃-C₈-cycloalkyl-C₁-C₆-alkyl, heterocyclyl-C₁-C₆-alkyl, 5- or 6-membered heteroaryl-C₁-C₆-alkyl, phenyl, naphthyl and a 3- to 10-membered saturated, partially unsaturated or aromatic mono- or bicyclic carbocyclic ring of heterocyclic, wherein the ring members of said heteroaryl of heteroaryl-C₁-C₆-alkyl and said mono- or bicyclic heterocyclic ring are selected from C, N, O and S and wherein one or more C ring members of the carbocylic ring or the heterocyclic ring may be replaced by one or more groups selected from C(═O) and C(═S); and wherein R^{12a} and R^{12b} are unsubstituted or substituted with one or more identical or different R^{12c}; wherein, R^{12c} is selected from the group consisting of halogen, cyano, nitro, oxo, hydroxy, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, —C(═O)—C₁-C₆-alkyl, C(═O)—C₁-C₆-alkoxy, C₁-C₆-alkylsulfonyl, hydroxy-C₁-C₆-alkyl, —C(═O)—NH₂, C(═O)—NH(C₁-C₆-alkyl), C₁-C₆-alkylthio-C₁-C₆-alkyl, amino-C₁-C₆-alkyl, C₁-C₆-alkylamino-C₁-C₆-alkyl, di-C₁-C₆-alkylamino-C₁-C₆-alkyl, aminocarbonyl-C₁-C₆-alkyl and C₁-C₆-alkoxy-C₁-C₆-alkyl; or
R^{12a} and R^{12b} together with the nitrogen atom to which they are bound form a saturated or partially unsaturated mono- or bicyclic 3- to 10-membered heterocyclic ring, wherein the ring members heterocyclic ring include beside one nitrogen atom, C, N, O and S(O)₀₋₂; and wherein one or more C atom of the heterocyclic ring may be replaced by one or more C(═O) and C(═S); and wherein the heterocyclic ring is unsubstituted or substituted with one or more identical or different groups R^{12d}; wherein R^{12d} is selected from the group consisting of halogen, cyano, nitro, oxo, hydroxy, sulfanyl, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, (C═O)—C₁-C₆-alkyl, C(═O)—C₁-C₆-alkoxy, C₁-C₆-alkylsulfonyl, hydroxyC₁-C₆-alkyl, C(═O)—NH₂, C(═O)—NH(C₁-C₆-alkyl), C₁-C₄-alkylthio-C₁-C₆-alkyl, amino-C₁-C₆-alkyl, C₁-C₆-alkylamino-C₁-C₆-alkyl, diC₁-C₆-alkylamino-C₁-C₆-alkyl, aminocarbonyl-C₁-C₆-alkyl and C₁-C₆-alkoxy-C₁-C₆-alkyl;
R¹³, R¹⁵ and R¹⁶ are independently selected from the group consisting of hydrogen, C₁-C₆-alkyl, C₁-C₆-alkoxy, C₁-C₆-alkoxy-C₁-C₆-alkyl, C₂-C₆-alkenyl, C₂-C₆-alkynyl, C₃-C₈-cycloalkyl, C₃-C₈-cycloalkenyl, —CH═N—O—C₁-C₆-alkyl, C(═O)—(C₁-C₆-alkyl), C(═O)—(C₁-C₆-alkoxy), C(═O)—(C₃-C₈-cycloalkyl), C(═O)-(phenyl), C(═O)-(heteroaryl), C₁-C₆-alkyl-C(═O)—(C₁-C₆-alkyl), C₁-C₆-alkyl-C(═O)—(C₁-C₆-alkoxy), C₁-C₆-alkoxyimino, C₁-C₆-alkoxyimino-C₁-C₆-alkyl, C₂-C₆-alkenyloxyimino-C₁-C₆-alkyl, C₂-C₆-alkynyloxyimino-C₁-C₆-alkyl, C₃-C₈-cycloalkyl-C₁-C₆-alkyl, aminocarbonyl-C₁-C₆-alkyl, C₁-C₆-alkylaminocarbonyl, C₁-C₆-alkylaminocarbonyl-C₁-C₆-alkyl, C₁-C₆-alkyl-NH—C(═O)(C₁-C₆-alkyl), C₁-C₆-alkyl-NH—C(═O)(C₃-C₈-cycloalkyl), C₁-C₆-alkyl-NH—C(═O)(phenyl), C₁-C₆-alkyl-NH—C(═O)—N(heteroaryl), C₁-C₆-alkyl-C(═O)—NH(C₁-C₆-alkyl), C₁-C₆-alkyl-C(═O)—N(C₁-C₆-alkyl)₂, di-C₁-C₆-alkyl-C(═O)—NH(C₃-C₆-cycloalkyl), C₁-C₆-alkyl-C(═O)—N(C₁-C₆-alkyl)(C₃-C₈-cycloalkyl), C₁-C₆-alkyl-C(═O)—NH(phenyl), C₁-C₆-alkyl-C(═O)—N(C₁-C₆-alkyl)(phenyl), C₁-C₆-alkyl-C(═O)—NH(heteroaryl), C₁-C₆-alkyl-C(═O)—N(C₁-C₆-alkyl)(heteroaryl), C₁-C₆-alkyl-C(═O)—NH(C₁-C₆-alkyl-C₃-C₈-cycloalkyl), C₁-C₆-alkyl-C(═O)—N(C₁-C₆-alkyl)(C₁-C₆-alkyl-C₃-C₈-cycloalkyl), C₁-C₆-alkyl-C(═O)—NH(C₁-C₆-alkyl-phenyl), C₁-C₆-alkyl-C(═O)—N(C₁-C₆-alkyl)(C₁-C₆-alkyl-phenyl), C₁-C₆-alkyl-C(═O)—NH(C₁-C₆-alkyl-heteroaryl), C₁-C₆-alkyl-C(═O)—N(C₁-C₆-alkyl)(C₁-C₆-alkyl-heteroaryl), C₁-C₆-alkylaminocarbonyl-C₃-C₈-cycloalkyl, C₃-C₈-cycloalkyl-C₁-C₆-alkyl, phenyl-C₁-C₆-alkyl, heteroaryl-C₁-C₆-alkyl, C₃-C₈-cycloalkyl-C₁-C₆-alkoxy, phenyl-C₁-C₆-alkoxy, heteroaryl-C₁-C₆-alkoxy, C₁-C₆-alkoxy-C₁-C₆-alkyl, C₃-C₈-cycloalkoxy-C₁-C₆-alkyl, phenoxy-C₁-C₆-alkyl, heteroaryloxy-C₁-C₆-alkyl, phenyl, naphthyl and a 3- to 10-membered saturated, partially unsaturated or aromatic mono- or bicyclic carbocyclic ring or heterocyclic ring, wherein the ring member atoms of said heteroaryls or said mono- or bicyclic heterocyclic ring include C, N, O and S(O)₀₋₂; wherein C ring member of the carbocyclic ring or the heterocyclic ring may be replaced by one or more C(═O) and C(═S); and wherein R¹³, R¹⁵ and R¹⁶ may be substituted or unsubstituted with one or more identical or different R^{15a},
R^{15a} is selected from the group consisting of halogen, cyano, hydroxy, oxo, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, NHSO₂—C₁-C₆-alkyl, (C═O)—(C₁-C₆-alkyl), C(═O)—(C₁-C₆-alkoxy), C₁-C₆-alkylsulfonyl, C₁-C₆-alkoxy-C₁-C₆-alkyl, hydroxy-C₁-C₆-alkyl, C(═O)—NH₂, C(═O)—NH(C₁-C₆-alkyl), C(═O)—N(C₁-C₆-alkyl)₂, —NH(C₁-C₆-alkyl), —N(C₁-C₆-alkyl)₂, C₁-C₆-alkylthio-C₁-C₆-alkyl, amino-C₁-C₆-alkyl, C₁-C₆-alkylamino-C₁-C₆-alkyl, di-C₁-C₆-alkylamino-C₁-C₆-alkyl and aminocarbonyl-C₁-C₆-alkyl; or
R¹⁵ is a 3- to 10-membered saturated, partially unsaturated or aromatic mono- or bicyclic carbocyclic or heterocyclic ring, wherein the ring members of the heterocyclic ring include C, N, O and S(O)₀₋₂ and the C ring members of the carbocyclic ring or the heterocyclic ring may be replaced by one or more C(═O) and C(═S); and wherein the carbocyclic ring and the heterocyclic ring are independently unsubstituted or substituted with one or more identical or different R^{15a}; or
R¹⁵ is phenyl or 5- or 6-membered heteroaryl, wherein the ring members of the heteroaryl ring include C, N, O and S; and wherein the phenyl and the heteroaryl rings are independently unsubstituted or substituted with one or more identical or different R^{15a};
or N-oxides, metal complexes, isomers, polymorphs or the agriculturally acceptable salts thereof.

2. The compound of claim 1,
wherein,
Het is Het-1, Het-2, Het-3, Het-4, Het-5, Het-6, Het-7 and Het-9;
R¹ is selected from the group consisting of CF₃, CHF₂, CF₂Cl, CF₂CF₃CH₂F, CH₂CF₃, CHClCF₃ and CCl₂CF₃;
L¹ is direct bond;
A is phenyl; and
L² is S(═O)₂, C(═O), L^{2a}, L^{2b}, L^{2c}, L^{2f} and L^{2g}.

3. The compound of claim 1, is selected from the group consisting of:
N-((4-fluorophenyl)(methyl)(oxo)-λ⁶-sulfaneylidene)-4-(5-(trifluoromethyl)-4,5-dihydro-1,2,4-oxadiazol-3-yl)benzamide; N-(4-(5-(difluoromethyl)-1,2,4-oxadiazol-3-yl)benzyl)-N-methoxybenzamide; N-(4-(5-(trifluoromethyl)-1H-1,2,4-triazol-3-yl)benzyl)pivalamide; 5-(difluoromethyl)-3-(4-(phenylsulfonyl)phenyl)-1,2,4-oxadiazole; N-((4-fluorophenyl)(methyl)(oxo)-λ⁶-sulfaneylidene)-4-(5-(trifluoromethyl)-4,5-dihydro-1,2,4-oxadiazol-3-yl)benzamide; N-((4-fluorophenyl)(methyl)(oxo)-λ⁶-sulfaneylidene)-4-(5-(trifluoromethyl)-1H-1,2,4-triazol-3-yl)benzamide; N-(methyl(oxo)(phenyl)-λ⁶-sulfaneylidene)-4-(5-(trifluoromethyl)-1H-1,2,4-triazol-3-yl)benzamide; 3-(4-(phenylsulfonyl)phenyl)-5-(trifluoromethyl)-1H-1,2,4-triazole; N-(methyl(oxo)(phenyl)-A-sulfaneylidene)-4-(5-(trifluoromethyl)-4,5-dihydro-1,2,4-oxadiazol-3-yl)benzamide; N-methyl-N-(2-phenoxyethyl)-4-(5-(trifluoromethyl)-1H-1,2,4-triazol-3-yl)benzamide; 3-(4-(phenylsulfonyl)phenyl)-5-(trifluoromethyl)-4,5-dihydro-1,2,4-oxadiazole; N-methyl-N-(2-phenoxyethyl)-4-(5-(trifluoromethyl)-4,5-dihydro-1,2,4-oxadiazol-3-yl)benzamide; (4-methoxyphenyl)(methyl)((4-(5-(trifluoromethyl)-1H-1,2,4-triazol-3-yl)phenyl)imino)-λ⁶-sulfanone; (4-methoxyphenyl)(methyl)((4-(5-(trifluoromethyl)-4,5-dihydro-1,2,4-oxadiazol-3-yl)phenyl)imino)-λ⁶-sulfanone; 4-(5-(difluoromethyl)-1,2,4-oxadiazol-3-yl)-N-methyl-N-(2-phenoxyethyl)benzamide; 4-(5-(difluoromethyl)-4,5-dihydro-1,2,4-oxadiazol-3-yl)-N-((4-fluorophenyl)(methyl)(oxo)-λ⁶-sulfaneylidene)benzamide; 2-phenyl-N-(4-(5-(trifluoromethyl)-4,5-dihydro-1,2,4-oxadiazol-3-yl)phenyl)propanamide; N-methyl-N-(4-(5-(trifluoromethyl)-4,5-dihydro-1,2,4-oxadiazol-3-yl)benzyl)cyclopropanecarboxamide; N-methyl-N-(4-(5-(trifluoromethyl)-4,5-dihydro-1,2,4-oxadiazol-3-yl)benzyl)cyclobutanecarboxamide; 2-phenyl-N-(4-(5-(trifluoromethyl)-4,5-dihydro-1,2,4-oxadiazol-3-yl)phenyl)acetamide; methyl(pyridin-2-yl)((4-(5-(trifluoromethyl)-4,5-dihydro-1,2,4-oxadiazol-3-yl)phenyl)imino)-λ⁶-sulfanone; N-((4-methoxyphenyl)(methyl)(oxo)-λ⁶-sulfaneylidene)-4-(5-(trifluoromethyl)-4,5-dihydro-1,2,4-oxadiazol-3-yl)benzamide; methyl((4-(5-(trifluoromethyl)-4,5-dihydro-1,2,4-oxadiazol-3-yl)phenyl)imino)(4-(trifluoromethyl)phenyl)-λ⁶-sulfanone; ((4-(5-(difluoromethyl)-4,5-dihydro-1,2,4-oxadiazol-3-yl)benzyl)imino)(methyl)(phenyl)-λ⁶-sulfanone; N-(2,4-difluorophenyl)-4-(5-(trifluoromethyl)-4,5-dihydro-1,2,4-oxadiazol-3-yl)benzamide; N-(4-chloro-2-fluorophenyl)-4-(5-(trifluoromethyl)-4,5-dihydro-1,2,4-oxadiazol-3-yl)benzamide; methyl(p-tolyl)((4-(5-(trifluoromethyl)-4,5-dihydro-1,2,4-oxadiazol-3-yl)benzyl)imino)-λ⁶-sulfanone; N-(4-(5-(difluoromethyl)-1,2,4-oxadiazol-3-yl)phenyl)-2-phenylacetamide; 1-methyl-5-(4-(phenylsulfonyl)phenyl)-3-(trifluoromethyl)-1H-1,2,4-triazole; (2-fluorophenyl)(methyl)((4-(1-methyl-3-(trifluoromethyl)-1H-1,2,4-triazol-5-yl)phenyl)imino)-λ⁶-sulfanone; 2-phenyl-N-(4-(5-(trifluoromethyl)-1H-1,2,4-triazol-3-yl)phenyl)acetamide; N-methyl-N-(4-(5-(trifluoromethyl)-1H-1,2,4-triazol-3-yl)benzyl)cyclopropanecarboxamide; N-methyl-N-(4-(5-(trifluoromethyl)-1H-1,2,4-triazol-3-yl)benzyl)cyclobutanecarboxamide; 2-phenyl-N-(4-(5-(trifluoromethyl)-1H-1,2,4-triazol-3-yl)phenyl)propanamide; methyl((4-(5-(trifluoromethyl)-1H-1,2,4-triazol-3-yl)phenyl)imino)(4-(trifluoromethyl)phenyl)-λ⁶-sulfanone; methyl(pyridin-2-yl)((4-(3-(trifluoromethyl)-1H-1,2,4-triazol-5-yl)phenyl)imino)-λ⁶-sulfanone; ((4-(3-(difluoromethyl)-1H-1,2,4-triazol-5-yl)benzyl)imino)(methyl)(phenyl)-λ⁶-sulfanone; N-((4-methoxyphenyl)(methyl)(oxo)-λ⁶-sulfaneylidene)-4-(3-(trifluoromethyl)-1H-1,2,4-triazol-5-yl)benzamide; N-(2,4-difluorophenyl)-4-(3-(trifluoromethyl)-1H-1,2,4-triazol-5-yl)benzamide; N-(4-chloro-2-fluorophenyl)-4-(3-(trifluoromethyl)-1H-1,2,4-triazol-5-yl)benzamide; methyl(pyridin-3-yl)((4-(5-(trifluoromethyl)-1H-1,2,4-triazol-3-yl)benzyl)imino)-λ⁶-sulfanone; N-methyl-4-(5-(perfluoroethyl)-1,2,4-oxadiazol-3-yl)-N-(2-phenoxyethyl)benzamide; ethyl 4-(5-(trifluoromethyl)isoxazol-3-yl)benzoate; 4-(5-(trifluoromethyl)isoxazol-3-yl)benzoic acid; N-(2,4-difluorophenyl)-4-(5-(trifluoromethyl)isoxazol-3-yl)benzamide; N-((4-fluorophenyl)(methyl)(oxo)-λ⁶-sulfaneylidene)-4-(5-(trifluoromethyl)isoxazol-3-yl)benzamide; N-(methyl(oxo)(4-(trifluoromethyl)phenyl)-A-sulfaneylidene)-4-(5-(trifluoromethyl)isoxazol-3-yl)benzamide; N-((4-chlorophenyl)(methyl)(oxo)-λ⁶-sulfaneylidene)-4-(5-(trifluoromethyl)isoxazol-3-yl)benzamide; N-(methyl(oxo)(pyridin-4-yl)-λ⁶-sulfaneylidene)-4-(5-(trifluoromethyl)isoxazol-3-yl)benzamide; N-(methyl(oxo)(pyridin-2-yl)-A-sulfaneylidene)-4-(5-(trifluoromethyl)isoxazol-3-yl)benzamide; N-((4-methoxyphenyl)(methyl)(oxo)-λ⁶-sulfaneylidene)-4-(5-(trifluoromethyl)isoxazol-3-yl)benzamide; N-(4-chloro-2-fluorophenyl)-4-(2-(trifluoromethyl)oxazol-4-yl)benzamide; N-methyl-N-(2-phenoxyethyl)-4-(5-(trifluoromethyl)isoxazol-3-yl)benzamide; N-(methyl(oxo)(phenyl)-λ⁶-sulfaneylidene)-4-(5-(trifluoromethyl)isoxazol-3-yl)benzamide; N-methyl-N-(2-phenoxyethyl)-4-(2-(trifluoromethyl)oxazol-4-yl)benzamide; N-(methyl(oxo)(pyridin-4-yl)-λ⁶-sulfaneylidene)-4-(2-(trifluoromethyl)oxazol-4-yl)benzamide; N-((4-fluorophenyl)(methyl)(oxo)-λ⁶-sulfaneylidene)-4-(2-(trifluoromethyl)oxazol-4-yl)benzamide; 4-(5-chloro-2-(trifluoromethyl)oxazol-4-yl)-N-(2,4-difluorophenyl)benzamide; 4-(5-chloro-2-(trifluoromethyl)oxazol-4-yl)-N-methyl-N-(2-phenoxyethyl)benzamide; N-((4-chlorophenyl)(methyl)(oxo)-λ⁶-sulfaneylidene)-4-(2-(trifluoromethyl)oxazol-4-yl)benzamide; 4-(5-chloro-2-(trifluoromethyl)oxazol-4-yl)-N-((4-fluorophenyl)(methyl)(oxo)-λ⁶-sulfaneylidene)benzamide; 4-(5-chloro-2-(trifluoromethyl)oxazol-4-yl)-N-(methyl(oxo)(phenyl)-λ⁶-sulfaneylidene)benzamide; 4-(5-chloro-2-(trifluoromethyl)oxazol-4-yl)-N-((4-methoxyphenyl)(methyl)(oxo)-λ⁶-sulfaneylidene)benzamide; N-(2,4-difluorophenyl)-4-(2-(trifluoromethyl)oxazol-4-yl)benzamide; N-((4-methoxyphenyl)(methyl)(oxo)-λ⁶-sulfaneylidene)-4-(2-(trifluoromethyl)oxazol-4-yl)benzamide; methyl 4-(2-(trifluoromethyl)oxazol-4-yl)benzoate; N-(2,6-difluorophenyl)-4-(2-(trifluoromethyl)oxazol-4-yl)benzamide; N-phenyl-4-(2-(trifluoromethyl)oxazol-4-yl)benzamide; N-methyl-N-phenyl-4-(2-(trifluoromethyl)oxazol-4-yl)benzamide; N-(methyl(oxo)(phenyl)-λ⁶-sulfaneylidene)-4-(2-(trifluoromethyl)oxazol-4-yl)benzamide; N-((2-fluorophenyl)(methyl)(oxo)-λ⁶-sulfaneylidene)-4-(2-(trifluoromethyl)oxazol-4-yl)benzamide; methyl 4-(5-chloro-2-(trifluoromethyl)oxazol-4-yl)benzoate; 4-fluoro-N-(4-(5-(trifluoromethyl)-4,5-dihydro-1,2,4-oxadiazol-3-yl)benzyl)benzenesulfonamide; N-(4-(3-(trifluoromethyl)-1,2,4-oxadiazol-5-yl)benzyl)cyclobutanecarboxamide; 1-isopropyl-3-(4-(3-(trifluoromethyl)-1,2,4-oxadiazol-5-yl)benzyl)urea; 1-isopropyl-3-(4-(3-(trifluoromethyl)-1H-1,2,4-triazol-5-yl)benzyl)urea; 4-fluoro-N-(4-(3-(trifluoromethyl)-1H-1,2,4-triazol-5-yl)benzyl)benzenesulfonamide; 1-isopropyl-3-(4-(5-(trifluoromethyl)-4,5-dihydro-1,2,4-oxadiazol-3-yl)benzyl)urea; 4-fluoro-N-(4-(3-(trifluoromethyl)-1,2,4-oxadiazol-5-yl)benzyl)benzenesulfonamide; methyl(phenyl)((4-(3-(trifluoromethyl)-1,2,4-oxadiazol-5-yl)benzyl)imino)-λ⁶-sulfanone; (4-methoxyphenyl)(methyl)((4-(5-(trifluoromethyl)isoxazol-3-yl)phenyl)imino)-λ⁶-sulfanone; methyl(phenyl)((4-(5-(trifluoromethyl)isoxazol-3-yl)phenyl)imino)-λ⁶-sulfanone; tert-butyl (4-(5-(trifluoromethyl)isoxazol-3-yl)phenyl)carbamate; methyl(pyridin-2-yl)((4-(5-(trifluoromethyl)isoxazol-3-yl)phenyl)imino)-λ⁶-sulfanone; (2-fluorophenyl)(methyl)((4-(5-(trifluoromethyl)-1,3,4-oxadiazol-2-yl)phenyl)imino)-λ⁶-sulfanone; methyl(phenyl)((4-(5-(trifluoromethyl)-1,3,4-oxadiazol-2-yl)phenyl)imino)-λ⁶-sulfanone; (4-methoxyphenyl)(methyl)((4-(5-(trifluoromethyl)-1,3,4-oxadiazol-2-yl)phenyl)imino)-λ⁶-sulfanone; methyl(pyridin-2-yl)((4-(5-(trifluoromethyl)-1,3,4-oxadiazol-2-yl)phenyl)imino)-λ⁶-sulfanone; isopropyl(methyl)((4-(5-(trifluoromethyl)-1,3,4-oxadiazol-2-yl)phenyl)imino)-λ⁶-sulfanone; tert-butyl (4-(5-(trifluoromethyl)-1,3,4-oxadiazol-2-yl)phenyl)carbamate; N-(4-(5-(trifluoromethyl)-1,3,4-oxadiazol-2-yl)phenyl)cyclopropanecarboxamide; N-(methyl(oxo)(phenyl)-λ⁶-sulfaneylidene)-4-(5-(trifluoromethyl)-1,3,4-oxadiazol-2-yl)benzamide; N-((4-fluorophenyl)(methyl)(oxo)-λ⁶-sulfaneylidene)-4-(5-(trifluoromethyl)-1,3,4-oxadiazol-2-yl)benzamide; N-((4-methoxyphenyl)(methyl)(oxo)-λ⁶-sulfaneylidene)-4-(5-(trifluoromethyl)-1,3,4-oxadiazol-2-yl)benzamide; tert-butyl (4-(1-methyl-5-(trifluoromethyl)-1H-pyrazol-3-yl)phenyl)carbamate; tert-butyl (4-(1-methyl-3-(trifluoromethyl)-1H-pyrazol-5-yl)phenyl)carbamate; N-(4-(1-methyl-3-(trifluoromethyl)-1H-pyrazol-5-yl)phenyl)cyclopropanecarboxamide; 1-isopropyl-3-(4-(1-methyl-5-(trifluoromethyl)-1H-pyrazol-3-yl)phenyl)urea; N-ethyl-N-methyl-4-(3-(trifluoromethyl)-1,2,4-oxadiazol-5-yl)benzenesulfonamide; and N-ethyl-N-methyl-4-(3-(trifluoromethyl)-1H-1,2,4-triazol-5-yl)benzenesulfonamide.

4. A combination comprising at least one compound of claim 1 and at least one further pesticidally active substance selected from the group consisting of fungicides, insecticides, nematicides, acaricides, biopesticides, herbicides, safeners, plant growth regulators, antibiotics, fertilizers and nutrients.

5. A composition comprising at least one compound of claim 1 and at least one agrochemically acceptable auxiliary.

6. The composition of claim 5, further comprising at least one additional active ingredient.

7. A composition comprising at least one compound of claim 1 and seed, wherein the amount of the compound is from 0.1 gai to 10 kgai per 100 kg of seeds.

8. A method for controlling or preventing phytopathogenic fungi, wherein the method comprises treating the fungi or the materials, plants, plant parts, locus thereof, soil or seeds to be protected against fungal attack, with an effective amount of at least one compound of claim 1.

9. A method for controlling or preventing infestation of plants by phytopathogenic micro-organisms in agricultural crops and or horticultural crops wherein an effective amount of at least one compound of claim 1, is applied to the seeds of plants.

10. Use of the compound of claim 1, for controlling or preventing plant diseases.

11. Use of the compound of claim 1, as fungicides and nematicides.

12. Use as claimed in claim 10, wherein the plant diseases are selected from Puccinia spp. (rusts), comprising P. triticina (brown or leaf rust), P. striiformis (stripe or yellow rust), P. hordei (dwarf rust), P. graminis (stem or black rust) and P. recondita (brown or leaf rust) on cereals viz., wheat, barley or rye; and Phakopsora spp. comprising Phakopsora pachyrhizi and P. meibomiae on soybeans, Hemileia vastatrix (Coffee rust), Uromyces spp., comprising U. fabae (rust of beans).

13. A process for preparing a compound of Formula I, wherein said process comprises any of the steps of:
step 1: 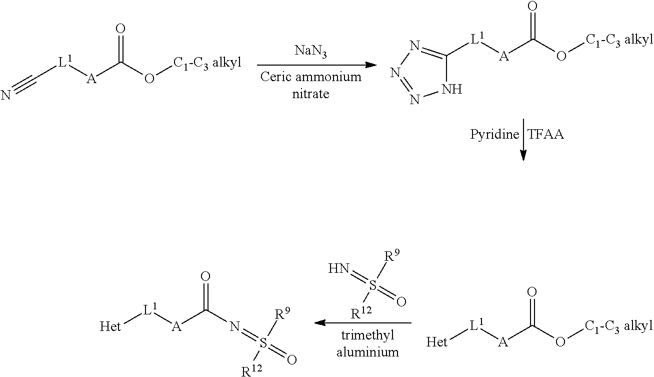
wherein, Het is 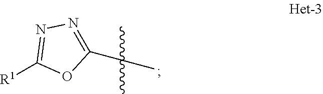
R¹ is CF₃; L¹ is a direct bond; and L² is 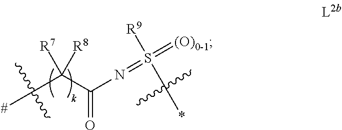
step 2: 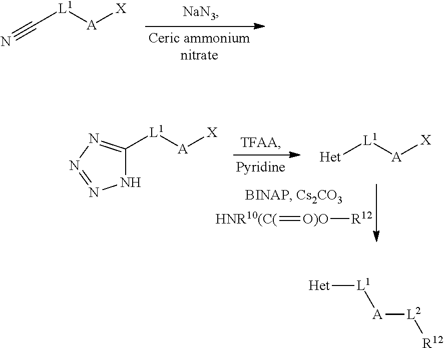
wherein, Het is 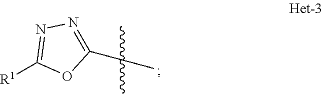
R¹ is CF₃; L¹ is a direct bond; L² is NR¹(C(═O))O—; and X is Cl, Br or I;
step 3: 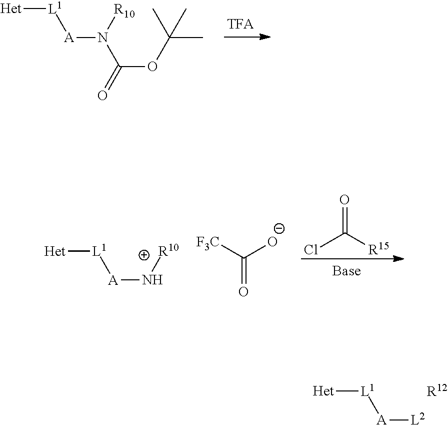
wherein, Het is 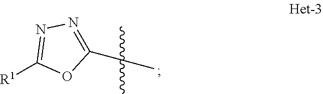
L¹ is a direct bond; and L² is —NR¹⁰;
step 4: 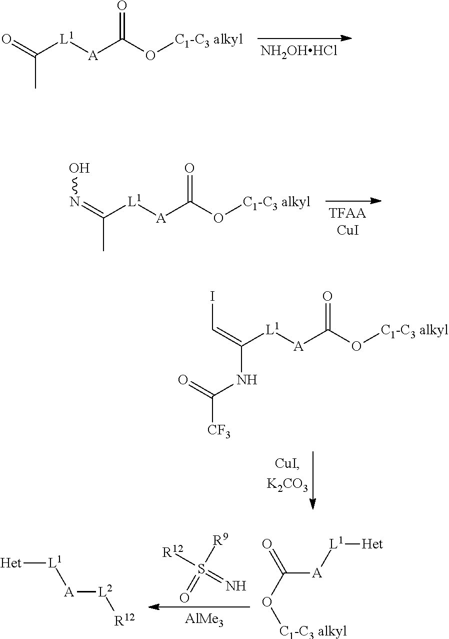
wherein, Het is 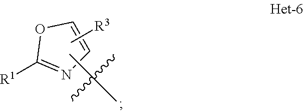
L¹ is a direct bond; and L² is 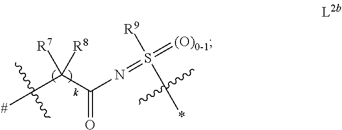
step 5: 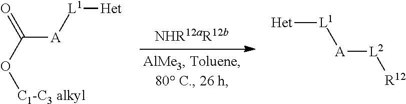
wherein, Het is 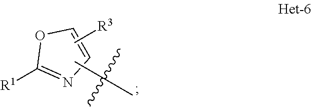
L¹ is a direct bond; and L² is —C(═O)—;
step 6: 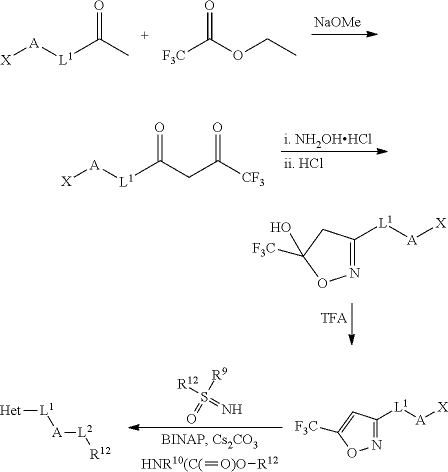
wherein, Het is 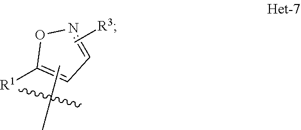
L¹ is a direct bond; L² is 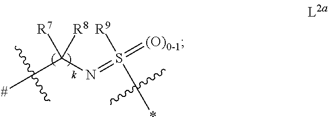
and X is Cl, Br or I;
step 7: 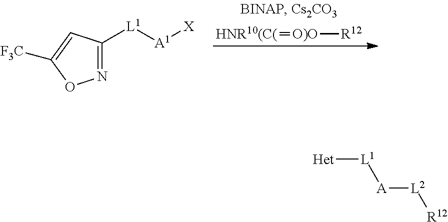
wherein, Het is 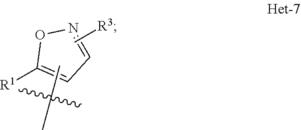
L¹ is direct bond; L² is NR¹⁰(C(═O))O—; and X is Cl, Br or I;
step 8: 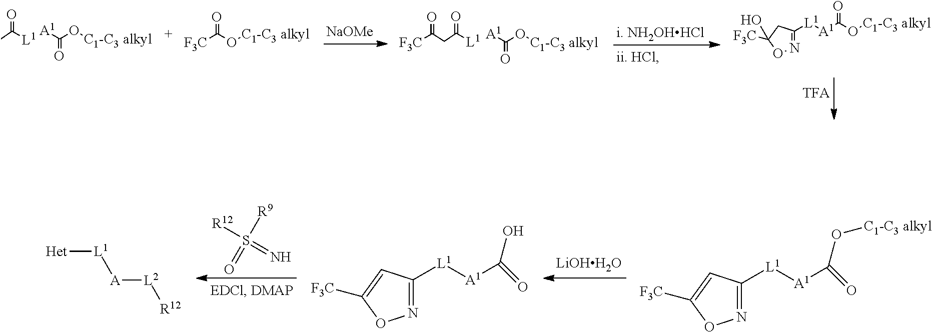
wherein, Het is 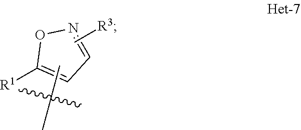
L¹ is a direct bond; and L² is 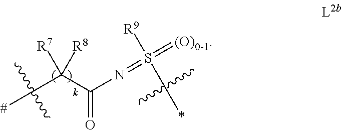
step 9: 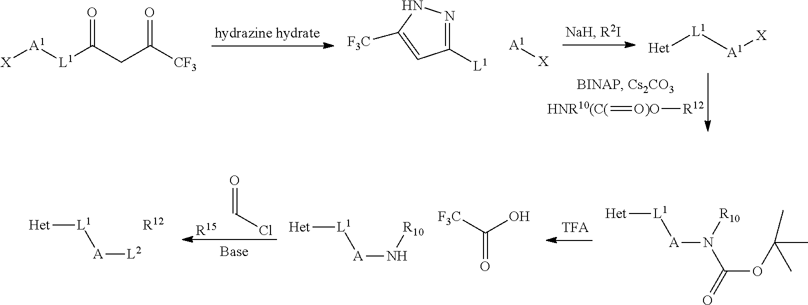
wherein, Het is 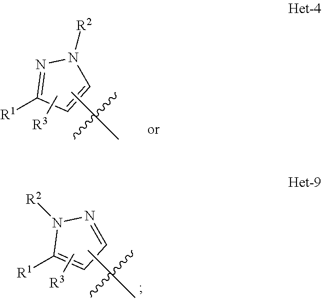
L¹ is a direct bond; L² is NR¹⁰(C(═O))O—; and X is Cl, Br or I;
step 10: 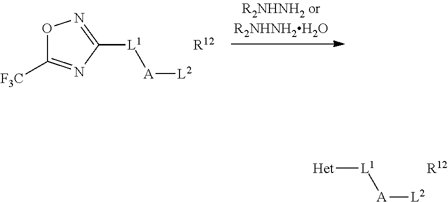
wherein, Het is 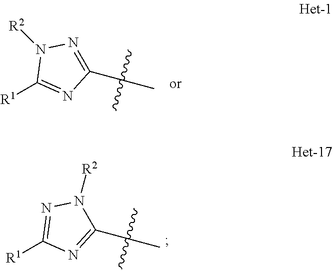
L¹ is a direct bond; and L² is —C(═O)—, —S(═O)₀₋₂—, —NR¹⁰—, 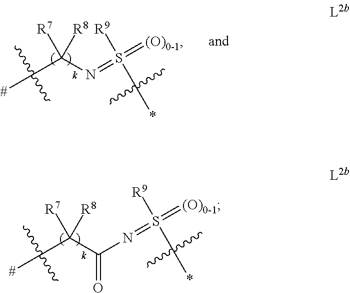
step 11: 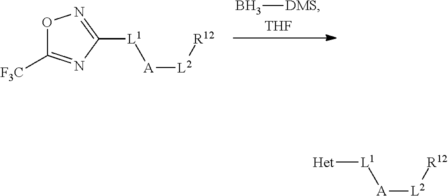
wherein, Het is 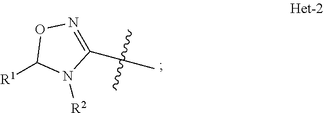
L¹ is a direct bond; and L² is —C(═O)—, —S(═O)₀₋₂—, —NR¹⁰—, or 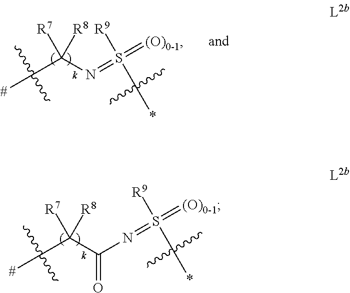
and
step 12: 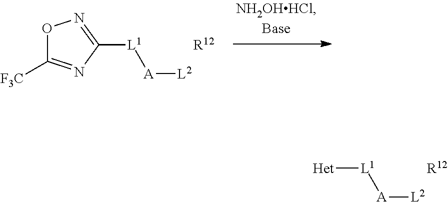
wherein, Het is 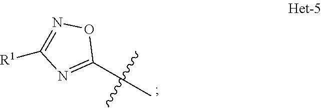
L¹ is a direct bond; and L² is —C(═O)—, —S(═O)₀₋₂—, —NR¹⁰—, 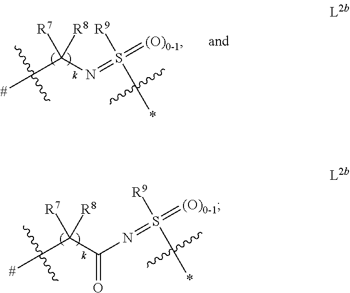
wherein, the definition of A, R¹, R², R³, R⁷, R⁸, R⁹, R¹⁰, R¹², R¹⁵, and k are as described in claim 1.
